SEQUENCE LISTING (170 sequences)

SEQ ID NO: 1 - DNA, 19 bp, Artificial
  Synthetic primer
gctgcggtaggtggttcaa

SEQ ID NO: 2 - DNA, 20 bp, Artificial
  Synthetic primer
ttgtcgcggatttgcaacta

SEQ ID NO: 3 - DNA, 19 bp, Artificial
  Synthetic primer
ggcgaaatggtgctggtaa

SEQ ID NO: 4 - DNA, 25 bp, Artificial
  Synthetic primer
ggccaagagatactcatagaacgtt

SEQ ID NO: 5 - DNA, 21 bp, Artificial
  Synthetic primer
acgcaatctagcagatgaagc

SEQ ID NO: 6 - DNA, 21 bp, Artificial
  Synthetic primer
tgtttggttggttattcgtgc

SEQ ID NO: 7 - DNA, 20 bp, Artificial
  Synthetic primer
attattcagaccgccggcag

SEQ ID NO: 8 - DNA, 20 bp, Artificial
  Synthetic primer
tgccgtcctgcaactgatgt

SEQ ID NO: 9 - DNA, 22 bp, Artificial
  Synthetic primer
ctctcaccctcaacccaatgtc

SEQ ID NO: 10 - DNA, 24 bp, Artificial
  Synthetic primer
tgtcggcggaatagtaataatgtt

SEQ ID NO: 11 - DNA, 20 bp, Artificial
  Synthetic primer
gcacattcaggcgggattag

SEQ ID NO: 12 - DNA, 24 bp, Artificial
  Synthetic primer
tctcttgttgggctgtatggtgta

SEQ ID NO: 13 - DNA, 28 bp, Artificial
  Synthetic primer
caaacggtatctgatgaaatgctggaag

SEQ ID NO: 14 - DNA, 28 bp, Artificial
  Synthetic primer
ttaaagctgcgcggaagaatagtgaaat

SEQ ID NO: 15 - DNA, 22 bp, Artificial
  Synthetic primer
cagaaagtgagggatttccata

SEQ ID NO: 16 - DNA, 27 bp, Artificial
  Synthetic primer
cacaaccattttcattatagttactgt

SEQ ID NO: 17 - DNA, 22 bp, Artificial
  Synthetic primer
ctcaaagcgaaggctttggtta

SEQ ID NO: 18 - DNA, 27 bp, Artificial
  Synthetic primer
ctgaagcgttttcattataattgctaa

SEQ ID NO: 19 - DNA, 24 bp, Artificial
  Synthetic primer
cgcaataggtgtatatattcttcc

SEQ ID NO: 20 - DNA, 24 bp, Artificial
  Synthetic primer
ctgaatagtttcgtatgccttctt

SEQ ID NO: 21 - DNA, 20 bp, Artificial
  Synthetic primer
acgatgctgcaggcaatggt

SEQ ID NO: 22 - DNA, 23 bp, Artificial
  Synthetic primer
catcagtattaccttctactaat

SEQ ID NO: 23 - DNA, 23 bp, Artificial
  Synthetic primer
taagagaattatgcagtgctgcc

SEQ ID NO: 24 - DNA, 21 bp, Artificial
  Synthetic primer
tccatagttgcctgactcccc

SEQ ID NO: 25 - DNA, 25 bp, Artificial
  Synthetic primer
cgtttgtatgatgttgatccagact

SEQ ID NO: 26 - DNA, 22 bp, Artificial
  Synthetic primer
tgtccatgtcttcaaaatgatg

SEQ ID NO: 27 - DNA, 20 bp, Artificial
  Synthetic primer
agatacctttggtcgtctgc

SEQ ID NO: 28 - DNA, 20 bp, Artificial
  Synthetic primer
cttacgcttctatctcggtg

SEQ ID NO: 29 - DNA, 27 bp, Artificial
  Synthetic primer
gcagtacagcagtttgttggactgacc

SEQ ID NO: 30 - DNA, 26 bp, Artificial
  Synthetic primer
gaatattttcattatcagtcccagtc

SEQ ID NO: 31 - DNA, 18 bp, Artificial
  Synthetic primer
gttcaacgactaggacgc

SEQ ID NO: 32 - DNA, 19 bp, Artificial
  Synthetic primer
taggcaccaatgtttcact

SEQ ID NO: 33 - DNA, 32 bp, Artificial
  Synthetic primer
gagtatagccagattatggcagttttattgtc

SEQ ID NO: 34 - DNA, 32 bp, Artificial
  Synthetic primer
ctccagcacttgcgctggaaacaacagacaac

SEQ ID NO: 35 - DNA, 31 bp, Artificial
  Synthetic primer
atggtgtgatttctcctagattggaaagtag

SEQ ID NO: 36 - DNA, 31 bp, Artificial
  Synthetic primer
cttctccaattgcttaccaagtgcaataacg

SEQ ID NO: 37 - DNA, 29 bp, Artificial
  Synthetic primer
cgacgtaacaaagaactaggtgcagaaac

SEQ ID NO: 38 - DNA, 30 bp, Artificial
  Synthetic primer
aagtatataaccacgctgtaaaactctgac

SEQ ID NO: 39 - DNA, 31 bp, Artificial
  Synthetic primer
gttagtcctgttttagatttatttggtgttg

SEQ ID NO: 40 - DNA, 30 bp, Artificial
  Synthetic primer
gagcagtcaataagatgagacgatagttag

SEQ ID NO: 41 - DNA, 22 bp, Artificial
  Synthetic primer
gatatccccggaataaatgaag

SEQ ID NO: 42 - DNA, 23 bp, Artificial
  Synthetic primer
catgaacaagaacgatatcaggc

SEQ ID NO: 43 - DNA, 31 bp, Artificial
  Synthetic primer
ctgttacttgttctggactctcgataattgg

SEQ ID NO: 44 - DNA, 31 bp, Artificial
  Synthetic primer
gcccactcctgttaaaatcctacccgcattg

SEQ ID NO: 45 - DNA, 26 bp, Artificial
  Synthetic primer
gtttattctatatacaaagaggctcc

SEQ ID NO: 46 - DNA, 26 bp, Artificial
  Synthetic primer
gcattgcaacaatcgctattctagat

SEQ ID NO: 47 - DNA, 25 bp, Artificial
  Synthetic primer
gcaacaaacggcgtgaaagtagttg

SEQ ID NO: 48 - DNA, 30 bp, Artificial
  Synthetic primer
caagatgaatatcactaccaataacaaaac

SEQ ID NO: 49 - DNA, 30 bp, Artificial
  Synthetic primer
cctacgggaggatataaaattatttttgag

SEQ ID NO: 50 - DNA, 31 bp, Artificial
  Synthetic primer
caaatacaccactataggctgttgagactaa

SEQ ID NO: 51 - DNA, 31 bp, Artificial
  Synthetic primer
ggacatgttcaggtgatttcccaatatagtg

SEQ ID NO: 52 - DNA, 32 bp, Artificial
  Synthetic primer
gattatgagtgtaatttattccaacttctccc

SEQ ID NO: 53 - DNA, 27 bp, Artificial
  Synthetic primer
gaaggcaatcaatgtgattgtgtcgcg

SEQ ID NO: 54 - DNA, 31 bp, Artificial
  Synthetic primer
cttcaaaatgaagattatagtacccttctac

SEQ ID NO: 55 - DNA, 32 bp, Artificial
  Synthetic primer
ctgttcagataggccatttacagctttaaatc

SEQ ID NO: 56 - DNA, 31 bp, Artificial
  Synthetic primer
cattccttttgtatatagtgctagttcatcc

SEQ ID NO: 57 - DNA, 32 bp, Artificial
  Synthetic primer
gataagtctgttgtggagacttaaaaagaatg

SEQ ID NO: 58 - DNA, 34 bp, Artificial
  Synthetic primer
ctttccagataattacaggtattcctgaagcaag

SEQ ID NO: 59 - DNA, 32 bp, Artificial
  Synthetic primer
cttaatagctctcattattctttttttaagcc

SEQ ID NO: 60 - DNA, 30 bp, Artificial
  Synthetic primer
ttatctgtaaaccatatcagcatctgaaac

SEQ ID NO: 61 - DNA, 33 bp, Artificial
  Synthetic primer
cgttcttttatctcactgtatagtatctttatg

SEQ ID NO: 62 - DNA, 30 bp, Artificial
  Synthetic primer
atgtttgaattaaagctaacgtaacaatcc

SEQ ID NO: 63 - DNA, 32 bp, Artificial
  Synthetic primer
ggaagttttcaaggatatgatagtggtggtgc

SEQ ID NO: 64 - DNA, 30 bp, Artificial
  Synthetic primer
ccgaataatatattcaatatattcctactc

SEQ ID NO: 65 - DNA, 29 bp, Artificial
  Synthetic primer
ttggaattttttaattagtggcttaccta

SEQ ID NO: 66 - DNA, 31 bp, Artificial
  Synthetic primer
catccgcttattaattgaagtaatctgaacc

SEQ ID NO: 67 - DNA, 31 bp, Artificial
  Synthetic primer
gatgccatgaatcaagcagtggctataaatc

SEQ ID NO: 68 - DNA, 30 bp, Artificial
  Synthetic primer
atcctcgtgtataatttcaggtatgccacc

SEQ ID NO: 69 - DNA, 30 bp, Artificial
  Synthetic primer
ggtgtagatttaccattagtgtcggcagac

SEQ ID NO: 70 - DNA, 31 bp, Artificial
  Synthetic primer
gaatttcttctttaagattcggatatttctc

SEQ ID NO: 71 - DNA, 34 bp, Artificial
  Synthetic primer
gaacatagtcgctattgtattttatttaaagcaa

SEQ ID NO: 72 - DNA, 33 bp, Artificial
  Synthetic primer
gactaggagcattattcctagagcgagtaaacc

SEQ ID NO: 73 - DNA, 32 bp, Artificial
  Synthetic primer
gcttttgtaagaggagattattttcacccaac

SEQ ID NO: 74 - DNA, 33 bp, Artificial
  Synthetic primer
caatccgactaagtcttcagtaaaaaactttac

SEQ ID NO: 75 - DNA, 32 bp, Artificial
  Synthetic primer
ctctatagaatggagtatataaactatggtta

SEQ ID NO: 76 - DNA, 34 bp, Artificial
  Synthetic primer
ccaaagaaaatactaacattatcacaatattggc

SEQ ID NO: 77 - DNA, 33 bp, Artificial
  Synthetic primer
ttcgtgatgataattccaatgatcaaacaagag

SEQ ID NO: 78 - DNA, 32 bp, Artificial
  Synthetic primer
gatgtaacaaatttgtagcgactaaggtctgc

SEQ ID NO: 79 - DNA, 31 bp, Artificial
  Synthetic primer
gagcaagagtttttcacctgacagcgagaag

SEQ ID NO: 80 - DNA, 33 bp, Artificial
  Synthetic primer
ctaaattcctgtaatttagctaaaactcttatc

SEQ ID NO: 81 - DNA, 28 bp, Artificial
  Synthetic primer
attagtacagctgctggaatatctcttc

SEQ ID NO: 82 - DNA, 26 bp, Artificial
  Synthetic primer
gatctagtgaacgtactattccaaac

SEQ ID NO: 83 - DNA, 37 bp, Artificial
  Synthetic primer
ctatctcagtcatctattgttaaagtttacgacggga

SEQ ID NO: 84 - DNA, 33 bp, Artificial
  Synthetic primer
gaacatagatgttgagacatcttttgtaatttc

SEQ ID NO: 85 - DNA, 31 bp, Artificial
  Synthetic primer
gcatcagtacagtgtgctaattggattgaag

SEQ ID NO: 86 - DNA, 30 bp, Artificial
  Synthetic primer
ctttaacatctgactttttctgttcccaac

SEQ ID NO: 87 - DNA, 32 bp, Artificial
  Synthetic primer
atacctacacaacttctgattatgcctttgtg

SEQ ID NO: 88 - DNA, 35 bp, Artificial
  Synthetic primer
gctcgataaacataatcaatatttgaaaaagtatg

SEQ ID NO: 89 - DNA, 18 bp, Artificial
  Synthetic primer
tggtagtgacagcaacga

SEQ ID NO: 90 - DNA, 19 bp, Artificial
  Synthetic primer
caaaggctaattcagcatc

SEQ ID NO: 91 - DNA, 24 bp, Artificial
  Synthetic primer
cattgccacgtgtcagctgcacat

SEQ ID NO: 92 - DNA, 27 bp, Artificial
  Synthetic primer
caccactcatcaaacgaatgagcgtgg

SEQ ID NO: 93 - DNA, 25 bp, Artificial
  Synthetic primer
tttgccgaaaacgcttgatacaggg

SEQ ID NO: 94 - DNA, 22 bp, Artificial
  Synthetic primer
tgcagagcgtcctttggtctat

SEQ ID NO: 95 - DNA, 25 bp, Artificial
  Synthetic primer
ctcttactcgtggtttccaacttga

SEQ ID NO: 96 - DNA, 24 bp, Artificial
  Synthetic primer
tggcgcccataagcaacactcgaa

SEQ ID NO: 97 - DNA, 25 bp, Artificial
  Synthetic primer
ctgaattgggcgattatctttatga

SEQ ID NO: 98 - DNA, 24 bp, Artificial
  Synthetic primer
acaatcaaactcaaccgaaagtga

SEQ ID NO: 99 - DNA, 21 bp, Artificial
  Synthetic primer
ggacgcctcggagtacctgag

SEQ ID NO: 100 - DNA, 23 bp, Artificial
  Synthetic primer
accgtggggtttctgaacttgtt

SEQ ID NO: 101 - DNA, 22 bp, Artificial
  Synthetic primer
ctggtgcagttcgcccgtgcca

SEQ ID NO: 102 - DNA, 21 bp, Artificial
  Synthetic primer
ggcgcagtatcagttcgactt

SEQ ID NO: 103 - DNA, 22 bp, Artificial
  Synthetic primer
ccttcgatatccttccctttcg

SEQ ID NO: 104 - DNA, 20 bp, Artificial
  Synthetic primer
tctgcgtccgtccctcggct

SEQ ID NO: 105 - DNA, 20 bp, Artificial
  Synthetic primer
agctgggattgcggtaacag

SEQ ID NO: 106 - DNA, 26 bp, Artificial
  Synthetic primer
caaagagtatcaccagcttcgactac

SEQ ID NO: 107 - DNA, 22 bp, Artificial
  Synthetic primer
ttgctgcgccaacaatcgcatc

SEQ ID NO: 108 - DNA, 20 bp, Artificial
  Synthetic primer
cggaagcaacgcgtaaactc

SEQ ID NO: 109 - DNA, 22 bp, Artificial
  Synthetic primer
gcgtcgcagaacattacattga

SEQ ID NO: 110 - DNA, 21 bp, Artificial
  Synthetic primer
acccgacgcgtccgatcacct

SEQ ID NO: 111 - DNA, 20 bp, Artificial
  Synthetic primer
atcctgagacaacactgaca

SEQ ID NO: 112 - DNA, 21 bp, Artificial
  Synthetic primer
ttgctggtgtttctattttca

SEQ ID NO: 113 - DNA, 25 bp, Artificial
  Synthetic primer
atcagaagagtcatactgccacttc

SEQ ID NO: 114 - DNA, 30 bp, Artificial
  Synthetic primer
aatttgtattttattcatgcctatatctgg

SEQ ID NO: 115 - DNA, 29 bp, Artificial
  Synthetic primer
ttagcggagataatttaaaatgatgacta

SEQ ID NO: 116 - DNA, 19 bp, Artificial
  Synthetic primer
ggtgcattcgcagcttcag

SEQ ID NO: 117 - DNA, 21 bp, Artificial
  Synthetic primer
gattgcgtaatgcaccgtgtt

SEQ ID NO: 118 - DNA, 28 bp, Artificial
  Synthetic primer
ttgtttataacaacgaagggactaacgt

SEQ ID NO: 119 - DNA, 151 bp, Neisseria meningitidis
agcgtgtgttggatgcggtggctgcggtaggtggttcaacggcaaatgtgcaggatacgaatgtgcagctgacacgtggcaatgtagtacgaactgttgccttggaagatttagttgcaaatccgcgacaaaatattttgctgcgtcgcgg

SEQ ID NO: 120 - DNA, 191 bp, Haemophilus influenzae
cacttgcagcattaatcgttggtgcattcgcagcttcagcagctaacgcagctgttgtttataacaacgaagggactaaagtagaattaggcggtcgtgtaagtattattgcagaacaaagcactagcactggagatgatcaaaaacatcaacacggtgcattacgtaatcagggttcacgtttccatatt

SEQ ID NO: 121 - DNA, 140 bp, Haemophilus influenzae
cgaaaaaatcgggattttagggcgaaatggtgctggtaaatccacgctcattcgtttgatgagtggtgttgagcctccaacaagtggtacgattgaacgttctatgagtatctcttggccgttagcttttagtggggcat

SEQ ID NO: 122 - DNA, 141 bp, Streptococcus pneumoniae
cgcctttatatcgaactcttacgcaatctagcagatgaagcaggtttgccgaaaacgcttgatacagggagtttagctggaattaaaacgcacgagtattgcacgaataaccaaccaaacaaccactcagaccatgtggat

SEQ ID NO: 123 - DNA, 121 bp, Adenovirus
ttaaaacagagaggaatttctgcagagcgtcctttggtctatatttcgagtgttgcttatgggcgccaagtctatctcaagttggaaaccacgagtaagagtgatgaagtagaggctgctt

SEQ ID NO: 124 - DNA, 136 bp, Adenovirus
tacatgcacatcgccgggcaggacgcctcggagtacctgagcccgggtctggtgcagtttgcccgcgccaccgacacgtacttcagcctgggcaacaagtttagaaaccccacggtggctcccacccacgatgtga

SEQ ID NO: 125 - DNA, 497 bp, Neisseria meningitidis
ctttaatttatacaccattactctcaccctcaacccaatgtctttctattggagagttaattattaacttaattcaaaaatattcaatggtggaaaacactgaaatgatccaagaacacttagagattattaagaaatttaattttattaatatactaaatgatttaaatggggtaataagtaaccctctctttaaaacagaagaaacatttgaaacacttcttaaatctgcagaattcgcatataaatctaaaaactactttcaggctattttttactggcaacttgccagcaaaaacaatattaccttattagggcataaagcattatggtactacaatgcactttataatgtaaaacaaatttataagatggaatattcagatattttttatatcgataatatctccgtagactttcatagtaaagataaattgacatgggaaaaaattaaacattattactattccgccgacaatagaattggtagagataga

SEQ ID NO: 126 - DNA, 482 bp, Neisseria meningitidis
atgaataccaccgtttttttgcacattcaggcgggattagcacaagccaatctattgctaaaattcaagacaaataccgcatatctcaaaatgactatatttttgtaagtcaacgctatcctgtttcagatgaagtttattataaaactattgtcgaaacattaaatcaaatgagtttgcgaatagaaggtaaaatttttattaaactccatcctaaagaaatggagaataaaaatattatgtctttattcctgaatatggtaacaattaatccccgtctagtagtgattaatgaacccccttttctcattgacccactaatttatttaacaacccctaaaggaattattggactgacatcgacttctattgtttatacacctttactttcccctactacccaatgtctgtcaattgggcaaatcgtgattgattcgatacaccatacagcccaacaagagaacgccgctttaattgaagag

SEQ ID NO: 127 - DNA, 738 bp, Neisseria meningitidis
tcgattatgttgactatcttcaaacggtatctgatgaaatgctggaagaaatgcattcccatttcaaaatcaaaaaagacaaattagtttttattccaaacatcacttatcccatttcattagaaaaaaaagaagctgatttctttattaaggataatgaagacatcgataatgctcagaaatttaaacgtatctctattgttggcagcattcagccaagaaaaaaccaattggatgccattaaaatcatcaataaaattaaaaatgaaaattacattttacagatatatggcaaatctattaataaagattactttgaattaattaaaaaatatattaaagacaataagttacaaaaccgtatcttattcaaaggtgaatcttccgagcaggaaatttatgaaaatacagatatcctgatcatgacatcagaaagtgagggatttccatatatatttatggaaggcatggtgtatgatattccaatcgttgtatatgattttaaatatggagcgaatgattacagtaactataatgaaaatggttgtgtttttaaaactggtgatatttctggaatggcaaaaaaaataattgagctattaaataacccagaaaaatataaagaattagttcaatataatcacaatcgcttcttaaaagaatatgcaaaagatgtggttatggctaaatatttcactattcttccgcgcagctttaataacgtatcattatcgtctg

SEQ ID NO: 128 - DNA, 160 bp, Neisseria meningitidis
agatatcctgatcatgacatcagaaagtgagggatttccatatatatttatggaaggcatggtgtatgatattccaatcgttgtatatgattttaaatatggagcgaatgattacagtaactataatgaaaatggttgtgtttttaaaactggtgatatt

SEQ ID NO: 129 - DNA, 160 bp, Neisseria meningitidis
agatatcctaatcatgacatctcaaagcgaaggctttggttatatatttctagagggtatggtgtacgatatccctatccttgcctataattttaaatatggagcgaatgattttagcaattataatgaaaacgcttcagtttttaaaactggtgatatt

SEQ ID NO: 130 - DNA, 435 bp, Neisseria meningitidis
aaaaaacttaacaatcaaaacgcaataggtgtatatattcttccttctaatcttactcttaagcctgcattatgtattctagaatcacataaagaagactttttaaataaatttcttcttactatttcctctgaaaatttaaagcttcaatacaaatttaatggacaaataaaaaatcctaagtccgtaaatgaaatttggacagatttatttagcattgctcatgttgacatgaaactcagcacagatagaactttaagttcatctatatctcaattttggttcagattagagttctgtaaagaagataaggattttatcttatttcctacagctaacagatattctagaaaactttggaagcactctattaaaaataatcaattatttaaagaaggcatacgaaactattcagaaatatcttcattaccctat

SEQ ID NO: 131 - DNA, 141 bp, Haemophilus influenzae
tcgttttacaaaagagttttctgaattgggcgattatctttatgagccagtgaagaagtactcatcagggatgaaagcccgacttgcatttgctctctcactttcggttgagtttgattgttatttaattgatgaggtgat

SEQ ID NO: 132 - DNA, 497 bp, Haemophilus influenzae
tcttacggatggcatgacagtaagagaattatgcagtgctgccataaccatgagtgataacactgctgccaacttacttctgacaacgatcggaggaccgaaggagcttaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcg

SEQ ID NO: 133 - DNA, 377 bp, Haemophilus influenzae
ataatctgcgttttatttgtcgtttgtatgatgttgatccagactacgttactcgttttacaaaagagttttctgaattgggcgattatctttatgagccagtgaagaagtactcatcagggatgaaagcccgacttgcatttgctctctcactttcggttgagtttgattgttatttaattgatgaggtgattgcagtaggggattcgcgttttgcagaaaaatgtaagtatgagttatttgagaaacgcaaagaccgttctatcattttagtttcacatagcccgagtgcgatgaaatcttattgtgataatgcggtagtgttagaaaatggtattatgcatcattttgaagacatggacaaagcgtatcaatac

SEQ ID NO: 134 - DNA, 283 bp, Haemophilus influenzae
tgggggcgaggctatacacaagatacctttggtcgtctgctaaatgatgcctttggtaaagaagtaaaaaacccattctattatgtcagaagttttactgatgatatgggtacatctgttcgccataacttcatcttagcaccacaaaacttctcattcttcgagcctatttttgcacaaaccccatacgacagtattcctgattactacgaagaaaaaggcagaattgaaccaattattaatcaccgagatagaagcgtaagcgatctcatttcggaagggt

SEQ ID NO: 135 - DNA, 169 bp, Streptococcus pneumoniae
ctctttgtttttagccggtcgttcaacgactaggacgctatttgagagtgtttcgacctatctaggaggctcaattcagcattttaatcagtatattgaaaatccattagatcctggtgaagtttttggcagtgaaacattggtgcctatattaaatatattaggggaa

SEQ ID NO: 136 - DNA, 683 bp, Streptococcus pneumoniae
atgataaaataattatttccgagtatagccagattatggcagttttattgtctagaattcattccaatgtatatatttataatggtccatactataatctatttaagatccccattattgaatctatgtacgattttctttttgtaagaatgctcaataagagaaccaagacagttttctgcaagacagaaaaggcaaagcattatcttaaaaataagggattcaatgattgtaaagtagtgggcgtaggtctagatgttgaaaagtttgaacaagaagaggaacctacggagaatactatagaacttttaaaaagaatggagaataagcaaaatattctttatgtaggctcactatcaaaaagaaaaaacacagctcatttaattagaatttttaatattttaaaatctaaaagtggtaagaaaaacgaacttcaattggtattgattggtaaggatgagggtaatattgttgaaaaaattaactattcacggtttaaagatgatattatttatcaaccttatttaaagaactctcagcttcaatttatttacccatcatcacaactatttgtgctcccgtcgattcaagagatttttggtatggtattacttgaggcaatgtattttaagttgtctgttgtttccagcgcaagtgctggaggagagacactgattcaagat

SEQ ID NO: 137 - DNA, 411 bp, Streptococcus pneumoniae
atatgaagatgtttcttcttatggtgtgatttctcctagattggaaagtagtaatggcctctatagtgttgatgcttttgtagagaaaccaaaaccagaagaagcgcctagccatttagctattattggacgttatctacttactcctgagattttttctatattagaaacccaaaagccaggagcaggtaatgaaattcaattgacagatgctattgatacattgaataagacacagagtgtttttgcgcgtgaatttgtgggcaaacgttacgatgttggtgataagtttaattttatgaaaacatcaattgattatgctcttcaacatcctcagattaaagagagtttaaaaaattacgttattgcacttggtaagcaattggagaagctagatgactgttcgtcaag

SEQ ID NO: 138 - DNA, 260 bp, Streptococcus pneumoniae
tatttgttatagatccgatacgacgtaacaaagaactaggtgcagaaactttttcgggaatttatgagatgcttgcaaaattaggatttgacaataatattataaaaggcttagaatggagagtgtctcctaattattattctttagggaatgtgtatactgcaattagacgttattattcagactttggtgtaattggtattgtaatttgtcagagttttacagcgtggttatatactttaggttatgaaaaagttaga

SEQ ID NO: 139 - DNA, 518 bp, Streptococcus pneumoniae
ctggacaacacaaagagatggttagtcctgttttagatttatttggtgttgtaccagattatgatttagaaattatgaaggctaaccaaaccttgttctctatcacaactagtatcttggaaaagataaaaccagttttagagaaggaacaaccagatattgtcctaattcacggtgacactacgacaacttatgcagcagccttggcagcattctatttgggaattaaagtaggacatgttgaagctggtttgcgaacgtacaatttacaaagtccatttcctgaagaatttaacaggcaatcgacatcaatcattgcaacttaccattttgctccaactgagttggctaaagaaaatctcttaaaagaaggtagagagaatgtttatgtgactggaaatactgtcattgatgctcttacaactactgttcaagaggattatacacacactcatttagatttaaacgctaacaatcgtctcatcttattgactgctcatagacgcgaaaatctcggc

SEQ ID NO: 140 - DNA, 547 bp, Streptococcus pneumoniae
ggaaaacgatggttgcatcagatatccccggaataaatgaagtagtcaataacaaaaatgggattcttgttcctgttaaggatgatgttgccttagcgagagcgattgaaaagcttgcgactgacaaaaaacttagagaaaaactcgcttatcaagcaaaaaaagactatgagacaaaatttaattattccttgtttttggataattatcggagactttatagaaaattaacgggagaatcaaaatgaaaaaagtcatgttggtttttgggacacgtccagaggctattaaaatgtgtcccctagtaaatgaactgaaacaaaatgattcaatcaagacacttgtgtgtgtaactggtcaacataaggaaatgctagagcaggttttggaggtgttcaaggttgtcccagatcatgatttgggtatcatgaaagcaaatcaaaccctattttccattacgattagtattttggataagattcaagcagttctagaacaagaaatgcctgatatcgttcttgttcatggagatacaacaacaacattt

SEQ ID NO: 141 - DNA, 470 bp, Streptococcus pneumoniae
ttaagttttatatttgctttctgttacttgttctggactctcgataattggaataatggttttcttatatctaattatgttccgtctatatttatatgggtttgctttctaattatttttcaaattactggttttattttacaaaaagttagtatatatgatttttctgtatggtatctgattttatcttatttttttatgtttggattaattttcaatgagtatatggggtttcaaacaactctgctgtggagccctagtaacttctataataatgaagaattatttcattcatatatttttataatttggattttgttttgttattctgtaggctatttatttttttatagtgatggaaaggtacattatcattcagaagtacaaaattatcaggaaaatgaagagaaaattttgtacaatgcgggtaggattttaacaggagtgggctttatttctagggtaataac

SEQ ID NO: 142 - DNA, 304 bp, Streptococcus pneumoniae
tatcttagttgtttttgtcggtttattctatatacaaagaggctccaatgtaattttgtttggaagaagtttgttagactgggacggatttacattagctactagttatggtgtaagatatacaggttttttagaatacgcaactttaaatggtcagttaattctttttttattaccgttaattagattgtttagatttagattttttacacaaactatcatttttgcttttcttctagaggttttggtactaagcaaatctagaatagcgattgttgcaatgcttatatatatagcatttgca

SEQ ID NO: 143 - DNA, 416 bp, Streptococcus pneumoniae
tgccaacaattttaatgttggcaacaaacggcgtgaaagtagttggaactgactacaatcaggatttggtgagaactttaaatgaaggtcaaacaacctttaaagaagatggattggatgaactattccataaagcagtggagtcgggtgtggactttacaaccgaatatcaacaaacggatacctatattatttccgttccaacaccatatgactccttctctaaaaaaattgatccaagctatgtgattgaagctacgaaaacggtacttgataattgcaataaaggagcggttattattattgaatcgaccgtatcaccaggaacggttgataaatttattcgacctgttgtagaagaaaaaggttttgttattggtagtgatattcatcttgtccatgctccagaacgtatt

SEQ ID NO: 144 - DNA, 866 bp, Streptococcus pneumoniae
tacctgagataagcactgttcctacgggaggatataaaattatttttgagtacgcaaataggttgtctgaacgaggtcatgagataacccttgtttttttaaccaataatgtatggaacagagttactaagaattgtaaaatcaaatcaattgtaggcaatatacgtggcaaaaaaaatccaagttggtttaaactgaaaccaactattcgtaaaataatgactccttatttagatgggagagattttcctgaagcagattttatatttgctactgcggttacaactgctaatattgtaaaagaaatgccagaaaaatatgggaaaaaatgttacctaatccaaggttttgaaacatggcttcttccagaaagtaaagttattgaaacttataattatggattccttaatattacagtatctaagtggttatgtgacatagtccaatcttatacagagactccagttttttgtgtatctaatccgattgatacagaaattttttatttgctaaatcctatagagaaacgaaacccatttcatctaggtatgttatatcatgaaggagagcataaaggaatctcttatgcgatagatgcaattaagaaagttaagaaaatttatccggaaatagaggttaatatttttggagttcctagtagaccagtatttttaccagaatattttaattatactcaacaagctacacaacaggaattacaaaagatatacaacgatacgtctatttttttgtgtgcaacaattgatgaaggctttggtttgacaggagcagagagtatggcttgtggctgtgcgttagtctcaacagcctatagtggtgtatttgaatatgcaattgatggcgag

SEQ ID NO: 145 - DNA, 503 bp, Streptococcus pneumoniae
tttttcatattacttttttaggacatgttcaggtgatttcccaatatagtgttatcggatttctcgtatcagcatattacttattagaagaaaagagaaatatattagtggcacagttgctgttgattttaacgattatcaattgttttttttcagatgtctctctctctaaagttattgctttgtttatgattgtttatattatttcatacaagttaagaaaactattctggaaaagaggaaggaagatatctgttgtaacttttattgcatctgttgtgatgttagcattagtaatttttggatattttttaccatatcttagatattttgattttacttttaatggtcgataccaaatttggcgcattgtgtatgctaccattcttcaagtgaaatggtttggatatggtttgtttggctttcaatttaaacttccgtggcagaaattgggagaagttggaataaattacactcataatcaagttttacagttagcactt

SEQ ID NO: 146 - DNA, 378 bp, Streptococcus pneumoniae
cttatctctagaatatctatgaaggcaatcaatgtgattgtgtcgcgttctttaatatttatattaattattctactcatagtaatattaaatggttttaagatttctgagacaagtttcgtctattattttgtattatttccgatttttatgatgattttgcagatgtactatgatgttaatgaaatcgcaaatctgatacggaaatttgttcgtataatatttcttttagcaattggctctctcctattttggcttattggtagtgtatttcatattatatccccaacggtttatgtgttgaattattggaatggtgggggaatagtagaagggtactataatcttcattttgaagcacaaaaaatagagattttg

SEQ ID NO: 147 - DNA, 1028 bp, Streptococcus pneumoniae
attggtttagtttatactatctgttcagataggccatttacagctttaaatctttttagaatttatatgtttcctgctgtaatatattttataataataaattgtaattttcgcaaggagagattactgatattgaagcaagcgcatgtatatactgcttgcatattagctttatggggaatttttcaggcgtgggtgttaaaagatcagtttttgattaaaataggatatccttcccaaggaaattttctaaagagtacagctttttatattggtggttttttcggtcaacaacgagttactagcactttttctgctcctaatttagctggagtttattttggaatatctttgatcattttactctctatatttgatactataaagagtaatagattggtattgttttcaattgtagcagcttttgttcttacatttagccgtagtgctattataagtactcttgtaggaatcgtattctttcaacgtaagaaattgttttcaacgacgaagattaatgttatgaccttggtaatcttccccttaatttttttgattgttttagttatattttatctttatcctgagaatgttattataaatatgctctatagcagttattcttcaacacttaatttgacagattcttcagcggttaagcatttggaagacttatggcttcctcttctaaaagttattgattatcctttgggacttggatttggtaataatggcccaattgttctgtcactctatcactcagcaaatttagtagaatcttctatttatcttcttgcatatgattttggtattttaggaatgttcatatatttgttcccttatttttatacgatttttgtatataaaaaatatttgcttagcggtgctatatgctgcttagttcttataacttatctatttcttccaaatgtagaaaattttgaaatcatattttttatttaccttttcattgggatggatgaactagcactatatacaaaaggaatgaatgtacatgaaattgatta

SEQ ID NO: 148 - DNA, 717 bp, Streptococcus pneumoniae
tgtgttgtaggttataattggataagtctgttgtggagacttaaaaagaatgatgtagttatttaccaacatccaatgtacggagtgcgtgttgcaaattttgcaattcctttattgaaaaagtataaaaatataaaatttatttcagtaatacatgatttagaatcgttaagaaaaggaatccaaggagtcattgaagataatgaaacaacaaatgctattgcagacaaagagttgttgtcaaaatttgataaagtgatttcccataatccgaaaatgacagaatatttagaagggatagggataaaaaaagaaaatttagttgaattgcaaatattcgactacctagatccatcagaaatagaagaaaagattgaagatggagtggttatagcggggaatctagcaaaaggaaaaagttcatatatatataagttgttagaaaatgaactgaacttcaaattaaatctttttgggccaaattttattaacgaagagctaccagaaaacgttgagtattttggtagtttacctccaaataaattgcctcaaaagctagtaggtaagtttggtttggtttgggacggtgatagtctagaaacttgtagtggaaatactggtaactatttgaagtataataatccacataaaacctcattatatcttgcttcaggaatacctgtaattatctggaaagaagctgcgttagcacagttt

SEQ ID NO: 149 - DNA, 613 bp, Streptococcus pneumoniae
tcttcagtatttcataatatcttaatagctctcattattctttttttaagccttaaacgaatcgtttttttgagtgtattaattatcataccagtatttttggtaatttattggtatgataaaaaagtaagcaaactagggaaagaacgaaaaattttaagtttattaaatatcttttccttaatatttataacaggaatattcttttatgtttatagtgtaaaatctgattttatatatacatttattcaagaacataatattaattcgatggctagaacagatttatggaagggagttgaatcaacctataatttcgcccctatatttatggggagagggatagggtttgtaacaaaatggatggataataattggatgactttgaatatcaatggtcttacaggatcaatggggatccataatgatattttgaagtactacattgagataggatttgtaggattatttatttatttttacactcttctttatagaaatgctaaacgtatatttgtaaaaattggtcataaagaatcattcatatattttgtattgataatgtttcagatgctgatatggtttacagataatatttcaatttaccataatt

SEQ ID NO: 150 - DNA, 614 bp, Streptococcus pneumoniae
tatatgtatcttatttaaatcgttcttttatctcactgtatagtatctttatgattgtattttacctttttcaaaatggacaggtattgttatattcacttggagtagagtatgattacttttatgttttaagatatgatgaaaccatcgtactacaatcagttattttttcaacacaatgtcttattgcagcctttatggctggagtcttttctacgaagaaagaggtatcaaaacccctttattcttatatggatcaattagaacgagaaaaactgattactactggaaagttattttggggtgcgtttgcgatttttgctttaccctttatgatgatgaagcttgttatcaccagtacatctgggtattttgcgatgatacgtttcgttggaagcctacctacaataacagtgttatttgaaaaaatgttcattgcttcaagtgtttttttgattgtttatttaaaatctgaggaaacttggagcaagtttttaaaagttgttatcttaggttggagtataatggctgctttaacaggagatagaaccgtaggtttagcagggattgttacgttagctttaattcaaacattaattggtaatcgtaaaaaa

SEQ ID NO: 151 - DNA, 741 bp, Streptococcus pneumoniae
ttattttattctatctatttggaagttttcaaggatatgatagtggtggtgcatttattatatttttactaatttatttaaaatatattttattcaaatttataattataaatataccgcaatccaatttaataagagcatttacaaacataggtattttaaatacggtcattttattagcagaagttttaagtcacggacatattaatttatttgtgaatcattatactttggcgcagaaaatagaaaccttaaataaggttgggacaaaccttgcggttttacgtggggggtttgaaaatcccttagtgacatctgtaatgctatcttctacattattattttttatgactattgagaaggctctcttacgtaatatactaattatgtctaacttatttttgattatggcaacagaaaaaagaacaggaattctaattagtatcgcgttgttgttctgttactatttcagaaaaaatttaaaaacaaagagtgttagtaagtttataataaaattcttgggaggattgtttttcttgggatttgctttattagccataaatatgataacaatttctggtcgtagcatttctcagatgataatagagaggttttcatcattaagtagtggttcagacttttctgctatacataggtcaatggcttttaaaataggcatagaaataatttggagtaggaatatattgaatatattattcggtaatggtttctactttttgc

SEQ ID NO: 152 - DNA, 536 bp, Streptococcus pneumoniae
atgtattctatatcaataatttggaattttttaattagtggcttacctattcaagtgttgttttcagatttgagtaaggcattcaattggatattagcagtatttttttataattattatttgaaaaatcccattaacgttgacaagataaagaaatatatgttttataatttcgctatattagttattattgttgctttattctatgttcaaagaggcgctaatgtagtattgtttggaagaagcttattaggttgggacggattcgtatcagctaccagttacggagtaagatatgcaggatttttagaatattcaacattaaatgggcagttgattctttttttgttaccgttaattaggttatttaaacttagtttttttacacaagtaactattcttgcttttttgctacaggttttagtattgagtaaatctagaatagctattattgctctgattatatacatagtatttgtagtaatggttcagattacttcaattaataagcggatgattgtagcgttttatccaac

SEQ ID NO: 153 - DNA, 334 bp, Streptococcus pneumoniae
aaggagcctatgatttaatagatgccatgaatcaagcagtggctataaatcctaatttgcatttgacaatggctggagatggagaacttgaagacatacgtcaaaagatatcaaatctgaatttgactgatcatattacaatatatgattgggttaatcagagagataaaaaaatactatttcaagctaatcagactttgatattgccttcttataatgaggggctcccgatggcaatattggaagctatggcttctggattagcaatcatatcaacgcctgtaggtggcatacctgaaattatacacgaggataatggctggttaattcaacc

SEQ ID NO: 154 - DNA, 668 bp, Streptococcus pneumoniae
gtatgttccatttgttatcaggtgtagatttaccattagtgtcggcagacaaattatttaaattctttgatcagaacaaatcaaataattttttgagcatggtctctgatgagattttgaaatcaaataaagtgtatgagcgtgtaaaatttagatacttatttcctagattcttagctagaaatattcagaacaaatatgttcgtaaatttgtagcatattaccgtaagctagagattaaaattcaacgtttaatgaaaatagattgttttaaaaaatataacatgagattgggttatgcatctaactgggtatctattaatcaggatttagttagaataatactagaagaagagaaaaatattgaaaaaatatttaaatattcgatagtaaatgatgaactgttcattcctacgataatgtataaatacaatttgatggaatcgttatattcatcttcaccgataacagatgccccaaatgattttcaaggaaatctaagatatatcaattggtgggatggagatcctcatacctggactgactctgagcatgatatagaacaattgaagcgtggtaaagctttaggtcataagttttctagaaaatttgatttagagaaatatccgaatcttaaagaagaaattctaattatcataaataggaca

SEQ ID NO: 155 - DNA, 557 bp, Streptococcus pneumoniae
ttcccatctatcttgatgatgaacatagtcgctattgtattttatttaaagcaaaataaaatacaatattggcaatggttattgttatctttatctgcttattggctgtatgatgaaacggattctcgattaacattttatagttcctgtatattgttgatatgcagtttattaataagatggatcccagaactattctctaagttcggatatatatttaaagcttttaaactcacctttattataaatgcagttgttagtttttgggtctcctttacctatctcaatttgagtcattcttatatcaataatctttttttaaaattagactatatattaggtggtcgtatatacttgatgaataaatcgttgaatttgtatggttttggattattcggacgaccagttgaatggaatggaaatggtctgaccattgaaggagttagaaactatcagacctatctgtatgttgataatttgtatgttcaaattttacaaaaatttggtttactcgctctaggaataatgctcctagtcctaactttaaccttgtttaa

SEQ ID NO: 156 - DNA, 448 bp, Streptococcus pneumoniae
tattttttattataaattatgcttttgtaagaggagattattttcacccaacaattatctttaactttacttttttgatagcaacactgttttgcattttaaatgctcaagagtatgctattgagttcaatggaggtacagtttttattgtgacgatttcaatgctcgtttttacaataataacagtattatcaaagaaaatatttaagactaatactttcacctttaagaataaattaaaatatctatatgtcagtaaaagtctaatttttacaataattattattcagattttaaatcttattttcttttatcgatacgaacaagctttattcagcgcttatgttggcggtcgtggaagtttctcgcaaataattaataactatgaccaattagtaaagttttttactgaagacttagtcggattgggggtcaggtctccgttttt

SEQ ID NO: 157 - DNA, 320 bp, Streptococcus pneumoniae
attctatttcttacccgctactctatagaatggagtatataaactatggttatacaaaagatttgattttcatgcaatggctggctttggcaactttgttgttaactctaccttctaaagccattgtgttaagcgaaaggaaaataaacctaaataacaataactatttattatttctcaataatatgaatacgtttatcacaacagtagcaattatatatctattaggaagtggttttagaaataaaggggaaatatattcaggtgccaatattgtgataatgttagtattttctttggtttattttatgattctcatt

SEQ ID NO: 158 - DNA, 733 bp, Streptococcus pneumoniae
ctcaacttgggaactactgattcgtgatgataattccaatgatcaaacaagagcaattattaaagaatacgaagaaaaagatagacggattaaattgattgaaaacatatcggaatatcatggagcctattataatttttgggggttattaaacgatgttagacaaaacgaaaatccatttgatttctatatgtttgctgatcaggatgatatctgggatgtggacaaattagaacgtttaattagttattactatagaaaagttaaaacagaagaaccggtattgatttacgctgatatgcggattatagatgctaacggtaaagtgatagctaatagtatggatcaattgatgggaattcggtataccaatcctatctcaacttttatggctcataaggtttatggatgtaatacgttatttaatcatgaattatttgaaatcttacccctccttccatgttatgctccagaattagccttcctatcacatgataactttacaacaaaaattgctgcattgaaagggcatgtgtatttttatgatgagcctactatgagttatagacgatatgggcataatgttacaagtaaacatgagtataactttacattgaagcgaatcttaaaacgtatctcgaaaattgatgaattagctaaagatcatgccttgacttacaagcagaccttagtcgctacaaatttgttacatcaacaaacaagtattgacact

SEQ ID NO: 159 - DNA, 554 bp, Streptococcus pneumoniae
ggatgaacttatttctaaaggagcaagagtttttcacctgacagcgagaagtaaaaatccgatacgttattattgggaaatgtatagattcttcaaagaatatgcatctgattatcaggcaatttgggttaatattaatagcttagcaaatattgactatttaaaatttgcaagatattttggtattccggttcgaattgtacacagccataatagtcaaaatatggataccaaacttcgtgaaaaattacataatcataatagagacaaaattgaaaagtgggcaaccgatttctgggcttgttcgcacgaagctgcaaaatggttttataacgaagaaactattggaaaagttaagattataccaaatgctattaatatgaatgcttctattttttcttctgaggcacgagataaaattcgaattgattatacattagacaataaatttgtgctaggtcatgtaggacgtttgcattttcagaagaatcaagaatttatgataagagttttagctaaattacaggaatttagagacgatgtctgcttagttt

SEQ ID NO: 160 - DNA, 474 bp, Streptococcus pneumoniae
cagtaaaaaaaatacgaataattagtacagctgctggaatatctcttctttttttaattgcctataaactcattgagatagttttacctgataatttgtatactttttttaatgttacaaaggcattatcatatgagaataggacagagtttgcgggacgaacaaatactatttccttcctatgggacaacttattttatcatgattatattagtgctatatttgggaaaggactaggttcatactctgttaattatatttatgaacttggtaaaatgcttgcagatggtggttttatttcagtgattttgctttattcatttttgttgtcattattcatacggggaactattactagaggaaaaaataagcaaagtgaaagactaattgtatccatcatagcttttgtggtgatgattagcattattgtttggaatagtacgttcactagatcaacttatcttgtatttttct

SEQ ID NO: 161 - DNA, 300 bp, Streptococcus pneumoniae
tggattggtgtaaaaaaggactatctcagtcatctattgttaaagtttacgacgggatcaagttacctcaagtatttggtgataaaaaatggtttagaaataaaaaaataaatattgtatttgttggtggttacgatattaaaaaaggtcaggagttgtttttaagctattttttaaaattaccaaaagaaatacaaatgcaatatactttgactttctatggaagtggaaaatctaagtatataaagaaattacaaaagatgtctcaacatctatgttctgatcaagttaaatttcata

SEQ ID NO: 162 - DNA, 394 bp, Streptococcus pneumoniae
ttcgttttgatagaggaaaagcatcagtacagtgtgctaattggattgaagaaaaaataaaaaacaataaagttattgattgaaataattatatttattgaatgataaataaggaggcctttagatgaaaagagttataacatatggtacatttgatttattgcattatggtcatatcaatcttttgaaacgtgctaaacagctaggtgattatttgattgtagttgtttcaagtgatgagtttaatttaaaagaaaagaataaagtatgttactttaactacgaacacagaaaaaatttagtagaagctattcgatatgtcgatttagtaatccctgaaactagttgggaacagaaaaagtcagatgttaaagagtaccgtattgacactttt

SEQ ID NO: 163 - DNA, 402 bp, Streptococcus pneumoniae
atatctggaagtttagtgggatacctacacaacttctgattatgcctttgtggatcctgttgggagtagtgtctattttttctagaattgacatggaacgatcattcctattttttttattaacaataggttgtttaattagcactattgctttgttagatatagttacgggagtatcttatgtctttaatggtttgtctcagcaactctatttggctgtgggaattctagttttaggctactggaatgctgatgtgattgttcattattggaaaatcatcaccatgacttttttgggagcatgtttgctgatttcagtggatatttattttcactactttcaaggacatactttttcaaatattgattatgtttatcgagctaagaattcagcggcatcta

SEQ ID NO: 164 - DNA, 217 bp, Streptococcus pneumoniae
tattaaaaaagagcatagactggtagtgacagcaacgacaatagtcatctctttactggcgttaacattttttttcaaacatataattaataatagtgaatcatatagccatcgagtgttaggtgttgtgaatttttttaaatattatgaatcagataggtttcatttgttttttggggatgctgaattagcctttggaaatacgacgaagggttat

SEQ ID NO: 165 - DNA, 290 bp, Streptococcus pneumoniae
ttgaattaccgaacataaataatttgtattttattcatgcctatatctgggggtgtacggcaggctttaatcatgcattgttagagatggttccttcagttgatattgataaagattatttatatatagaaaaactggctcatgatagttattttgcaaagtttgcactagagtatgggaaggtgttgttctgccctgagcaactggtcttgtatcgaagacacggacataatgtaacaactagtcatcattttaaattatctccgctaaatattctcagaaaggctatt

SEQ ID NO: 166 - DNA, 290 bp, Streptococcus pneumoniae
ttgaattaccgaacataaataatttgtattttattcatgcctatatctgggggtgtacggcaggctttaatcatgcattgttagagatggttccttcagttgatattgataaagattatttatatatagaaaaactgtctcatgataattattttgcaaagtttgcactagagtatgggaaggtgttgttctgccctgagcaactggtcttgtatcgaagacacggacataatgtaacaactagtcatcattttaaattatctccgctaaatattctcagaaaggctatt

SEQ ID NO: 167 - DNA, 115 bp, Klebsiella pneumoniae
gcgcagaactttgaagcggtggcgcagtatcagttcgacttcggtctgcgtccgtccctcggctatgtgctgtcgaaagggaaggatatcgaaggggtggggagtgaagatctgg

SEQ ID NO: 168 - DNA, 124 bp, Lysteria monocytogenes
aaagcaactatcgcggctacagctgggattgcggtaacagcatttgctgctccaacaatcgcatccgcaagcactgtagtagtcgaagctggtgatactctttggggtatcgcacaaagtaaag

SEQ ID NO: 169 - DNA, 107 bp, Escherichia coli
ggaatatttcgcgccactggcggaagcaacgcgtaaactcgacccgacgcgtccgatcacctgcgtcaatgtaatgttctgcgacgctcacaccgataccatcagcg

SEQ ID NO: 170 - DNA, 118 bp, Streptococcus agalactiae
tgcagatatcaatcttatttatcctgagacaacactgacagtaacttacgatcagaagagtcatactgccacttcaatgaaaatagaagcaccagcaacaaatgctgctggtcaaaca

(19) United States
(12) Patent Application Publication
(10) Pub. No.: US 2010/0167272 A1
(43) Pub. Date: Jul. 1, 2010

(54) Method for Detecting and Analyzing Pathogens in a Sample

(75) Inventors: Chiara Azzari, Firenze (IT); Massimo Resti, Firenze (IT); Maria Moriondo, Bagno a Ripoli (IT)

Correspondence Address: LUCAS & MERCANTI, LLP, 475 PARK AVENUE SOUTH, 15TH FLOOR, NEW YORK, NY, 10016, US

(73) Assignee: UNIVERSITA' DEGLI STUDI DI FIRENZE, Firenze (IT)

(21) Appl. No.: 12/668,830

(22) PCT Filed: Jul. 11, 2008
(86) PCT No.: PCT/IB2008/001817

§ 371 Date: Feb. 2, 2010

(30) Foreign Application Priority Data
Jul. 13, 2007 (IT) MI2007A001410

(51) Int. Cl. C12Q 1/70; C12Q 1/68

(52) U.S. Cl. 435/5; 435/6

(57) ABSTRACT

The present invention relates to a method and kits thereof for detecting the presence and/or the specific serogroup of a prokaryote selected from the group consisting of & pneumionae, N. meningitidis, H. influenzae, Adenovirus, Klebsiella Pneumonite, Lysteria monocytogenes, Escherichia coli and Streptococcus agalactiae in a sample taken from a human being, through amplification of specific target regions.

### FIELD OF THE INVENTION

The present invention relates to a method for detecting the presence and/or the specific serogroup of a prokaryote selected from the group consisting of S. pneumoniae, N. meningitidis, H. influenzae, Adenovirus, Klebsiella Pneumonite, Lysteria monocytogenes Escherichia coli and Streptococcus agalactiae in a sample taken from a human being.

### PRIOR ART

Infections with Streptococcus pneumonite (pneumococcus), Neisseria meningitidis (meningococcus) and Haemophilus influenzae are among the most dangerous for humans. S. pneumoniae was discovered as an etiologic agent of pneumonia, but it also causes other pathologies, among which are meningitis, pericarditis, osteomyelitis and peritonitis. Bacteria are gram-positive and express polysaccharide capsules that are present in a variety of 90 serotypes. Once present in a human being, pneumococci may penetrate the blood system (causing bacteraemia) and be delivered to meninxes, joints, bones and the peritoneal cavity provoking meningitis, cerebral abscess, septic arthritis, or osteomyelitis. N. meningitidis is a prokaryote which is pathogenic only for human beings and causes both meningitis, a lethal pathology well known in the art, and a septicaemia that may be lethal and may lead to pathologies like the Waterhouse-Friderichsen syndrome.

H. influenzae is a gram-negative coccobacillus discovered for the first time in 1892 during a flu pandemic and it is responsible for several pathologies, some of which are flu-like, but not of flu in a direct manner. H. influenzae determines immune and phlogistic reactions which may cause other pathologies, such as e.g. epiglottitits. The pathogenesis mechanism for H. influenzae is not well known, but it is widely known that it causes or is responsible for several pathologies, although there exist strains of H. influenzae which do not cause their guest any pathology.

There exist antibiotic therapies to diminish or eliminate the presence of said pathogenic prokaryotes in human beings and the pathologies deriving therefrom. However, said prokaryotes are considered to be very dangerous because the pathologies connected thereto may become lethal/disabling (e.g. they may cause paralysis) more quickly than the therapies used to heal said infections and the consequent pathologies. Indeed, the virulence and lethality of pathologies like meningitis are so high that it is the physicians' common practice to administer antibiotic therapies even prior to confirming, by means of experimental analyses, a suspicion for meningitis in a patient.

Laboratory analyses used in the known art to diagnose the presence of these pathogenic, and possibly lethal, prokaryotes make use of microbiological culture techniques. Said analyses may last up to 36 hours before they provide a diagnostic outcome. It was also noted that the use of antibiotics prior to the diagnosis with said techniques may provide a false-negative result, since the pathogen does not grow in the culture due to the presence of the antibiotic.

Further, culture diagnostic techniques require that the pathogenic prokaryote in the sample be capable of reproducing (in vivo), that the culture media be suitable for selecting every species of specific pathogen, and finally that the period between the sampling and the culture be minimum in order to keep the pathogen alive. As a consequence, microbiologic equipment is needed close to where the samples are taken and the resulting costs are high.

Further to these diagnostic issues, there exists, as an additional problem, the medical need to be able to diagnose the serogroup and/or specific serotype of said prokaryote pathogens. S. pneumoniae, as mentioned above, is differentiated in 90 serotypes. In order to evaluate the action and/or the outcome of a vaccination programme in the prevention of infective pathologies caused by said prokaryote pathogens, a method needs to be developed that solves the diagnosis problems mentioned above and that exactly determines the serogroup and/or serotype of the prokaryote species of the pathogen. The above-mentioned pathogens, such as e.g. S. pneumoniae, are divided into several serogroups. Each serogroup is divided into serotypes. Knowing the serotype is more important than knowing the serogroup. For example, for serogroup 19 of the pathogen S. pneumoniae there exist two serotypes, 19A and 19F. Serotype 19F is present in the vaccine and serotype 19A is not. As a consequence it is important to diagnose whether the serotype present in an infection is serotype 19A or 19F. It is therefore preferable, in diagnostics, to obtain the serotype, and only if that is not possible (because it is not known in the art) or useful, then the serogroup. In the context of the present invention, by serogroup it is also meant serotype, where that is possible thanks to the knowledge of the art. The method is directed to pathogenic prokaryotes and therefore only the primers for serotypes and/or serogroups known in the art to be infective are used therein. The authors of the present invention have implemented a method for detecting the presence and/or the serogroup of a pathogen of fast execution, high selectivity, efficiency and versatility without the use of culture techniques. The sample may advantageously be treated to extract or render accessible the DNA present therein.

It is therefore an object of the invention a method for detecting the presence and/or the serogroup of a pathogen selected from the group consisting of N. meningitidis, H. influenzae, S. pneumoniae or Adenovirus in a biological sample, comprising the following steps:
a) incubating a first aliquot of the sample under conditions such as to enable the amplification and the revelation of specific target regions of the genoma of said pathogens, if present in the sample, wherein the target regions are comprised in:
SEQ ID 119 of the ctrA gene of N. meningitidis, and
SEQ ID 120 of the P2 gene or SEQ ID 121 of the bex gene of H. influenzae and
SEQ ID 122 of the lyt gene or SEQ ID 123 of the ply gene of S. pneumoniae and
SEQ ID 124 of Adenovirus;
   and/or,
b) if the sample is positive for N. meningitidis, incubating a second aliquot of the sample under conditions such as to enable the amplification of specific serotyping target regions of the genoma of N. meningitidis, wherein the target regions are comprised in:
SEQ ID 125 for serotype B of N. meningitidis,
SEQ ID 126 for serotype C of N. meningitidis,
SEQ ID 127 for serotype W135 of N. meningitidis,
SEQ ID 128 for serotype W of N. meningitidis,
SEQ ID 129 for serotype Y of N. meningitidis,
SEQ ID 130 for serotype A of N. meningitidis,
c) revealing the amplification;
b′) if the sample is positive for H. influenzae, incubating a second aliquot of the sample under conditions such as to enable the amplification of region SEQ ID 131 of the genoma of H. influenzae for revealing capsulated H. influenzae, and
c′) revealing the amplification;
d′) if the sample is positive for the revelation of capsulated H. influenzae, incubating a third aliquot of the sample under conditions such as to enable the amplification of specific serotyping target regions of the genoma of H. influenzae wherein the target regions are comprised in:
SEQ ID 132 for the revelation of H. influenzae that are productors of beta-lactamase
SEQ ID 133 for the revelation of H. influenzae serotypes a, b, c, d, e, f;
SEQ ID 134 for the revelation of H. influenzae B type capsulated serotype; b″) if the sample is positive for S. pneumonite, incubating a second aliquot of the sample under conditions such as to enable the amplification of specific serotyping target regions of the genoma of S. pneumoniae, wherein the target regions are comprised in:
SEQ ID 135 for serotype 19F of S. pneumoniae
SEQ ID 136 for serotype 22F of S. pneumoniae
SEQ ID 137 for serotype 3 of S. pneumoniae
SEQ ID 138 for serotype 6 of S. pneumoniae
SEQ ID 139 for serotype 19A of S. pneumoniae
SEQ ID 140 for serotype 9v of S. pneumoniae
SEQ ID 141 for serotype 4 of S. pneumoniae
SEQ ID 142 for serotype 14 of S. pneumoniae
SEQ ID 143 for serotype 12f of S. pneumoniae
SEQ ID 144 for serotype 7f of S. pneumoniae
SEQ ID 145 for serotype 11a of S. pneumoniae
SEQ ID 146 for serotype 33f of S. pneumoniae
SEQ ID 147 for serotype 16f of S. pneumoniae
SEQ ID 148 for serotype 35b of S. pneumoniae
SEQ ID 149 for serotype 18f of S. pneumoniae
SEQ ID 150 for serotype 38 of S. pneumoniae
SEQ ID 151 for serotype 31 of S. pneumoniae
SEQ ID 152 for serotype 15c of S. pneumoniae
SEQ ID 153 for serotype 8 of S. pneumoniae
SEQ ID 154 for serotype 10A of S. pneumoniae
SEQ ID 155 for serotype 35f of S. pneumoniae
SEQ ID 156 for serotype 34 of S. pneumonite
SEQ ID 157 for serotype 1 of S. pneumoniae
SEQ ID 158 for serotype 17f of S. pneumoniae
SEQ ID 159 for serotype 20 of S. pneumoniae
SEQ ID 160 for serotype 15a of S. pneumoniae
SEQ ID 161 for serotype 7c of S. pneumoniae
SEQ ID 162 for serotype 18f of S. pneumoniae
SEQ ID 163 for serotype 5 of S. pneumoniae
SEQ ID 164 for serotype 23F of S. pneumoniae
c″) highlighting the amplification;
d″) if the sample is positive for serotype 6 of S. pneumoniae, incubating a third aliquot of the sample under conditions such as to enable the amplification of the regions SEQ ID 165 for serotype 6a or SEQ ID 166 for serotype 6b of S. pneumoniae
e″) highlighting the amplification.

Preferably, the target regions are comprised in:
from nt. 21 to nt 131 of SEQ ID 119 of the ctrA gene of N. meningitidis, and
from nt 21 to nt 171 of SEQ ID 120 of the P2 gene or from nt 21 to nt 120 of SEQ ID 121 of the bex gene of H. influenzae and
from nt 21 to nt 121 of SEQ ID 122 of the lyt gene or from nt 21 to nt 101 of SEQ ID 123 of the ply gene of S. pneumoniae and
from nt 21 to nt 116 of SEQ ID 124 of Adenovirus.

Preferably, if the sample is positive for N. meningitidis, the specific serotyping target regions of the genoma of N. meningitidis are comprised in:
from nt 21 to nt 477 of SEQ ID 125 for serotype B of N. meningitidis,
from nt 21 to nt 462 of SEQ ID 126 for serotype C of N. meningitidis,
from nt 21 to nt 718 of SEQ ID 127 for serotype W135 of N. meningitidis,
from nt 21 to nt 140 of SEQ ID 128 for serotype W of N. meningitidis,
from nt 21 to nt 140 of SEQ ID 129 for serotype Y of N. meningitidis,
from nt 21 to nt 415 of SEQ ID 130 for serotype A of N. meningitidis,
if the sample is positive for H influenzae, the target region for revealing capsulated H. influenzae is comprised within the region from nt 21 to nt 121 of SEQ ID 131;
if the sample is positive for the revelation of capsulated H. influenzae, the specific serotyping target regions are comprised in:
from nt 21 to nt 477 of SEQ ID 132 for the revelation of H. influenzae that are productors of beta-lactamase
from nt 21 to nt 357 of SEQ ID 133 for the revelation of H. influenzae serotypes a, b, c, d, e, f;
from nt 21 to nt 263 of SEQ ID 134 for the revelation of H. influenzae B type capsulated serotype;
if the sample is positive for S. pneumonite, the specific serotyping target regions of the genoma of S. pneumoniae are comprised in:
from nt 21 to nt 149 of SEQ ID 135 for serotype 19F of S. pneumoniae
from nt 21 to nt 663 of SEQ ID 136 for serotype 22F of S. pneumoniae
from nt 21 to nt 391 of SEQ ID 137 for serotype 3 of S. pneumoniae
from nt 21 to nt 240 of SEQ ID 138 for serotype 6 of S. pneumoniae
from nt 21 to nt 498 of SEQ ID 139 for serotype 19A of S. pneumoniae
from nt 21 to nt 527 of SEQ ID 140 for serotype 9v of S. pneumoniae
from nt 21 to nt 350 of SEQ ID 141 for serotype 4 of S. pneumoniae
from nt 21 to nt 284 of SEQ ID 142 for serotype 14 of S. pneumoniae
from nt 21 to nt 396 of SEQ ID 143 for serotype 12f of S. pneumoniae
from nt 21 to nt 846 of SEQ ID 144 for serotype 7f of S. pneumoniae
from nt 21 to nt 483 of SEQ ID 145 for serotype 11a of S. pneumoniae
from nt 21 to nt 358 of SEQ ID 146 for serotype 33f of S. pneumoniae
from nt 21 to nt 1008 of SEQ ID 147 for serotype 16f of S. pneumoniae
from nt 21 to nt 697 of SEQ ID 148 for serotype 35b of S. pneumoniae
from nt 21 to nt 593 of SEQ ID 149 for serotype 18f of S. pneumoniae
from nt 21 to nt 594 of SEQ ID 150 for serotype 38 of S. pneumoniae
from nt 21 to nt 721 of SEQ ID 151 for serotype 31 of S. pneumoniae
from nt 21 to nt 516 of SEQ ID 152 for serotype 15c of S. pneumoniae
from nt 21 to nt 314 of SEQ ID 153 for serotype 8 of S. pneumoniae
from nt 21 to nt 648 of SEQ ID 154 for serotype 10A of S. pneumoniae
from nt 21 to nt 537 of SEQ ID 155 for serotype 35f of S. pneumoniae
from nt 21 to nt 428 of SEQ ID 156 for serotype 34 of S. pneumonite
from nt 21 to nt 300 of SEQ ID 157 for serotype 1 of S. pneumoniae
from nt 21 to nt 713 of SEQ ID 158 for serotype 17f of S. pneumoniae
from nt 21 to nt 534 of SEQ ID 159 for serotype 20 of S. pneumoniae
from nt 21 to nt 454 of SEQ ID 160 for serotype 15a of S. pneumoniae
from nt 21 to nt 280 of SEQ ID 161 for serotype 7c of S. pneumoniae
from nt 21 to nt 374 of SEQ ID 162 for serotype 18f of S. pneumoniae
from nt 21 to nt 382 of SEQ ID 163 for serotype 5 of S. pneumoniae
from nt 21 to nt 197 of SEQ ID 164 for serotype 23F of S. pneumoniae
if the sample is positive for serotype 6 of S. pneumoniae, the target regions enabling the discrimination between serotype 6a and 6b are, from nt 21 to nt 270 of SEQ ID 165 and from nt 21 to nt 270 of SEQ ID 166, respectively.

Preferably, the amplification and revelation of the specific regions comprised in SEQ ID 119 of the ctrA gene of N. meningitidis, and in SEQ ID 122 of the lyt gene or in SEQ ID 123 of the ply gene of S. pneumoniae occurs in a single first reaction environment; and the amplification and the revelation of the specific regions comprised in SEQ ID 120 of the P2 gene or in SEQ ID 121 of the bex gene of H. influenzae, and in SEQ ID 124 of Adenovirus occurs in a single second reaction environment.

Preferably, if the sample is positive for S. pneumoniae, the second aliquot is incubated under conditions such as to enable the amplification of specific serotyping target regions of the genoma of S. pneumoniae,
in a single first reaction environment for the sequences comprised in:
SEQ ID 135 for serotype 19F, SEQ ID 138 for serotype 6, SEQ ID 140 for serotype 9v, SEQ ID 141 for serotype 4, SEQ ID 142 for serotype 14, SEQ ID 163 for serotype 5;
in a single second reaction environment for the sequences comprised in:
SEQ ID 136 for serotype 22F, SEQ ID 138 for serotype 6, SEQ ID 137 for serotype 3, SEQ ID 139 for serotype 19A;
in a single third reaction environment for the sequences comprised in:
SEQ ID 140 for serotype 9v, SEQ ID 141 for serotype 4, SEQ ID 142 for serotype 14, SEQ ID 143 for serotype 12f;
in a single fourth reaction environment for the sequences comprised in:
SEQ ID 144 for serotype 7f, SEQ ID 145 for serotype 11A, SEQ ID 146 for serotype 33f;
in a single fifth reaction environment for the sequences comprised in:
SEQ ID 147 for serotype 16f, SEQ ID 148 for serotype 35b, SEQ ID 149 for serotype 18f, SEQ ID 150 for serotype 38;
in a single sixth reaction environment for the sequences comprised in:
SEQ ID 151 for serotype 31, SEQ ID 152 for serotype 15c, SEQ ID 153 for serotype 8, SEQ ID 154 for serotype 10A;
in a single seventh reaction environment for the sequences comprised in:
SEQ ID 155 for serotype 35f, SEQ ID 156 for serotype 34, SEQ ID 157 for serotype 1,

SEQ ID 158 for serotype 17f;
in a single eighth reaction environment for the sequences comprised in:
SEQ ID 159 for serotype 20, SEQ ID 160 for serotype 15°, SEQ ID 161 for serotype 7c, SEQ ID 162 for serotype 18f;
in a single ninth reaction environment for the sequences comprised in:
SEQ ID 163 for serotype 5, SEQ ID 164 for serotype 23F.

More preferably, the reaction of amplification and revelation of step a) occurs by RT-PCR.

Still preferably, the reactions of amplification and revelation of the steps from b) to e″) occur by PCR and revelation of the amplificate by chromatography.

More preferably, the sample is not pre-incubated to increase the pathogen load.

It is a further object of the invention a kit for detecting the presence and/or the serogroup of a pathogen selected from the group consisting of N. meningitidis, H. influenzae, S. pneumoniae o Adenovirus in a biological sample comprising primer and probe oligonucleotides capable of amplifying the target reasons cited above and control target regions.

Preferably, the primers and probes for N. meningitidis and S. pneumoniae are in a single first reaction environment and the primers and probes for H. influenzae and Adenovirus are in a single second reaction environment.

Even more preferably:
the primers for SEQ ID 119 are SEQ ID 1 and SEQ ID 2 and the probe is SEQ ID 91;
the primers for SEQ ID 120 are SEQ ID 116 and SEQ ID 117 and the probe is SEQ ID 118; o the primers for SEQ ID 121 are SEQ ID 3 and SEQ ID 4 and the probe is SEQ ID 92;
the primers for SEQ ID 122 are SEQ ID 5 and SEQ ID 6 and the probe is SEQ ID 93; o the primers for SEQ ID 123 are SEQ ID 94 and SEQ ID 95 and the probe is SEQ ID 96;
the primers for SEQ ID 124 are SEQ ID 99 and SEQ ID 100 and the probe is SEQ ID 101.

Preferably, for the amplification of specific serotyping target regions of the genoma of N. meningitidis:
the primers for SEQ ID 125 are SEQ ID 9 and SEQ ID 10;
the primers for SEQ ID 126 are SEQ ID 11 and SEQ ID 12;
the primers for SEQ ID 127 are SEQ ID 13 and SEQ ID 14;
the primers for SEQ ID 128 are SEQ ID 15 and SEQ ID 15;
the primers for SEQ ID 129 are SEQ ID 17 and SEQ ID 18;
the primers for SEQ ID 130 are SEQ ID 19 and SEQ ID 20.
the primers for the control region are SEQ ID 1 and SEQ ID 2, or SEQ ID 7 and SEQ ID 8.

Preferably, for the amplification of specific serotyping target regions of the genoma of H. Influenzae:
the primers for SEQ ID 131 are SEQ ID 97 and SEQ ID 98;
the primers for SEQ ID 132 are SEQ ID 23 and SEQ ID 24;
the primers for SEQ ID 133 are SEQ ID 25 and SEQ ID 26;
the primers for SEQ ID 134 are SEQ ID 27 and SEQ ID 28;
the primers for the control region are SEQ ID 21 and SEQ ID 22.

Preferably, for the amplification of specific serotyping target regions of the genoma of S. pneumoniae:
the primers for SEQ ID 135 are SEQ ID 31 and SEQ ID 31;
the primers for SEQ ID 136 are SEQ ID 33 and SEQ ID 34;
the primers for SEQ ID 137 are SEQ ID 35 and SEQ ID 36;
the primers for SEQ ID 138 are SEQ ID 37 and SEQ ID 38;
the primers for SEQ ID 139 are SEQ ID 39 and SEQ ID 40;
the primers for SEQ ID 140 are SEQ ID 41 and SEQ ID 42;
the primers for SEQ ID 141 are SEQ ID 43 and SEQ ID 44;
the primers for SEQ ID 142 are SEQ ID 45 and SEQ ID 46;
the primers for SEQ ID 143 are SEQ ID 47 and SEQ ID 48;
the primers for SEQ ID 144 are SEQ ID 49 and SEQ ID 50;
the primers for SEQ ID 145 are SEQ ID 51 and SEQ ID 52;
the primers for SEQ ID 146 are SEQ ID 53 and SEQ ID 54;
the primers for SEQ ID 147 are SEQ ID 55 and SEQ ID 56;
the primers for SEQ ID 148 are SEQ ID 57 and SEQ ID 58;
the primers for SEQ ID 149 are SEQ ID 59 and SEQ ID 60;
the primers for SEQ ID 150 are SEQ ID 61 and SEQ ID 62;
the primers for SEQ ID 151 are SEQ ID 63 and SEQ ID 64;
the primers for SEQ ID 152 are SEQ ID 65 and SEQ ID 66;
the primers for SEQ ID 153 are SEQ ID 67 and SEQ ID 68;
the primers for SEQ ID 154 are SEQ ID 69 and SEQ ID 70;
the primers for SEQ ID 155 are SEQ ID 71 and SEQ ID 72;
the primers for SEQ ID 156 are SEQ ID 73 and SEQ ID 74;
the primers for SEQ ID 157 are SEQ ID 75 and SEQ ID 76;
the primers for SEQ ID 158 are SEQ ID 77 and SEQ ID 78;
the primers for SEQ ID 159 are SEQ ID 79 and SEQ ID 80;
the primers for SEQ ID 160 are SEQ ID 81 and SEQ ID 82;
the primers for SEQ ID 161 are SEQ ID 83 and SEQ ID 84;
the primers for SEQ ID 162 are SEQ ID 85 and SEQ ID 86;
the primers for SEQ ID 163 are SEQ ID 87 and SEQ ID 88;
the primers for SEQ ID 164 are SEQ ID 89 and SEQ ID 90;
the primers for SEQ ID 165 are SEQ ID 114 and SEQ ID 115;
the primers for SEQ ID 166 are SEQ ID 114 and SEQ ID 115;
the primers for the control region are SEQ ID 29 and SEQ ID 30,
and wherein said primers are, optionally, partially grouped in a plurality of reaction environments.

It is a further object of the invention a for detecting the presence and the serogroup of a pathogen selected from the group consisting of N. meningitidis, H. influenzae, S. pneumoniae or Adenovirus in a biological sample comprising the kits cited above.

It is a further object of the invention to provide a method for detecting the presence and/or the serogroup of a pathogen selected from the group consisting of Klebsiella pneumoniae, Lysteria monocytogenes, E. coli, S. agalactiae in a biological sample comprising the following steps:
a) incubating an aliquot of the sample under conditions such as to enable the amplification and revelation of specific target regions of the genoma of said pathogens, if present in the sample, wherein the target regions are comprised in:
SEQ ID 167 of the phoE gene of Klebsiella pneumoniae,
SEQ ID 168 of the iap gene of Lysteria monocytogenes,
SEQ ID 169 of the uidA gene of E. coli,
SEQ ID 170 of the sip gene of S. agalactiae.

Preferably the target regions are comprised in:
from nt 21 to nt 95 of SEQ ID 167 of the phoE gene of Klebsiella pneumoniae,
from nt 21 to nt 104 of SEQ ID 168 of the iap gene of Lysteria monocytogenes,
from nt 21 to nt 87 of SEQ ID 169 of the uidA gene of E. coli,
from nt 21 to nt 98 of SEQ ID 170 of the sip gene of S. agalactiae.

Preferably the amplification and revelation of the specific regions comprised in SEQ ID 167 of the phoE gene of Klebsiella pneumoniae, and in SEQ ID 169 of the uidA gene of E. coli occurs in a single first reaction environment; and the amplification and revelation of the specific regions comprised in SEQ ID 168 of the iap gene of Lysteria monocytogenes, and in SEQ ID 170 of the sip gene of S. agalactiae occurs in a single second reaction environment.

Preferably, the sample is not pre-incubated to increase the pathogen load.

It is still an object of the invention a kit for detecting the presence and/or the serogroup of a pathogen selected from the group consisting of Klebsiella pneumoniae, Lysteria monocytogenes, E. coli, S. agalactiae in a biological sample comprising primer and probe oligonucleotides capable of amplifying the target regions cited above.

Preferably the primers and probes for Klebsiella pneumonia and E. coli are in a single first reaction environment and the primers and probes for Lysteria monocytogenes and S. agalactiae are in a single second reaction environment.

Preferably:
the primers for SEQ ID 167 are SEQ ID 102 and SEQ ID 103 and the probe is SEQ ID 104;
the primers for SEQ ID 168 are SEQ ID 105 and SEQ ID 106 and the probe is SEQ ID 107;
the primers for SEQ ID 169 are SEQ ID 108 and SEQ ID 109 and the probe is SEQ ID 110;
the primers for SEQ ID 170 are SEQ ID 111 and SEQ ID 112 and the probe is SEQ ID 113.

Preferred embodiments of the invention are reported in the claims below.

The sample taken from the human body may be any type of human biological tissue. Among these, blood, pleuric liquid and cefalorachidian liquor (C SF) are preferred. In the cases wherein an organ pathology (meningitis, pneumonia with pleuritis) is present, it is preferred that a sample be taken from the seat of infection (CSF or pleuric liquid). When these are not available and for the unlocalised forms (sepsis, bacteriaemia), whole blood is preferred, because the standard regulations for the sampling thereof and the displacement/freezing of the same are well known and practiced in the art, with equipment also known to the skilled in the art.

An advantage of the method according to the present invention is that a sample of a biological tissue may be used, even if this has been sampled far away or however within a time interval greater than 1 day, which is the maximum time for using the samples by culture. Samples taken even 8 days earlier are still usable in the method according to the invention. The sample taken may optionally be frozen and defrosted at the time of analysis.

Thus, with a method of freezing and defrosting of the sample, the presence and/or the serogroup and/or the serotype of a pathogen may be detected more than 8 days later and for an indefinite time. The only requirement of the method according to the invention is that the nucleic acid of the prokaryote remain intact and extractable and have not hydrolised or decomposed during the wait between the sampling and the beginning of the method.

The biological sample taken may be either test tube or absorbed as a spot on blotting paper, the blotting paper sheets being similar to those normally used for the Guthri test at birth, for the screening of hypothyroidism and phenylketonuria. An advantage of this method is that blood may be sampled also from a fingertip by capillary prick. Thus the sampling may be done also at the patient's place of residence or in the doctor's office, without resorting to sampling of venous blood. The sheet used for the spot (which is sterile prior to use), once used, must immediately be put away in a plastic sachet in order to avoid contaminations by environment germs and subsequently sent to the laboratory carrying out the test.

In the contest of the method of the present invention, it is preferred to extract and use only DNA because RNA is labile and not preservable for a long time. Any method for extracting DNA from a sample of biological tissue known in the art may be used for the method of the invention. It is preferable to use a group of reagents or kits for extracting DNA comprising K protease, such as e.g. the QIAmp DNA mini kit, by Qiagen, Hilden, Germany. The use of K protease has shown, in comparative studies, a greater capability of retrieving bacterial DNA and said capability results in a greater sensitivity of the test.

When the germ is sought in a blood sample absorbed on a cardboard sheet the same extracting method may be used, but this has to be preceded by a step wherein a fraction of the cardboard sheet of about 15-20 mm² (square millimetres) is dissolved in a volume from 100 to 200 micro-litres of water or of other lysis buffers, one of which is the lysis buffer present in the Qiagen extracting kit for biological liquids.

It is another advantage of the method according to the invention that to perform the diagnosis only the presence of the nucleic acid, preferably DNA, extracted from the sample, is needed. All the methods known in the art to maintain nucleic acid sequences, preferably DNA sequences, stable in a solution for a certain time are incorporated.

In an aspect of the invention, the presence of a specific sequence of said nucleic acid, preferably DNA, selected based on its being present solely in one of the pathogens to be assayed, is determined.

In the context of the present invention, with the term “pathogen” there is meant one or more prokaryotes selected from the group consisting of N. meningitidis, S. pneumonite, H. influenzae and Adenovirus, or, if the sample comes from newborn subjects, Klebsiella pneumoniae, Lysteria monocytogenes, E. coli, S. agalactiae.

Said determination of the presence of the sequence belonging to one of said pathogens is carried out by amplification, preferably through use of primer sequences, with at least one Real-Time Polymerase Chain Reaction (RT-PCR). RT-PCR is a technique known in the art. Reaction amplification is signalled and, possibly, quantified by use of a fluorescent probe which generates a signal simultaneously with the amplification reaction The nucleic acid sequences resulting from the RT-PCR reaction are DNA sequences. The RT-PCR reaction may be carried out with any equipment or reagent known in the art.

In a preferred embodiment of said aspect of the invention wherein the presence or absence of at least a pathogen is detected, use is made of primer and hydrolysis probe sequences, among which, e.g., the TaqMan probes may be used in a PCR amplification cycle reaction known as TaqMan reaction. Said TaqMan probe sequence is a fluorescent probe bonded by means of a nucleic acid sequence to a quencher. The nucleic acid sequence is DNA. The TaqMan probe sequence is hydrolised thanks to the polymerisation of Taq-polyiinerase (through the action of 3′-5′ exonuclease of Taq polymerase). As a consequence, the fluorescent hydrolisis probe or TaqMan probe, without interferences from the quencher, emits fluorescence to signal that polymerization has occurred. TaqMan primers and probes used in the present invention may be built according to methods known in the art to build DNA sequences and methods to bind probes or quenchers thereto.

In said aspect of the invention, the RT-PCR reaction may detect the presence of one or more sequences, and consequently, if the reagents are adequately selected, one or more pathogen species at the same time. An advantage of the use of said aspect of the invention, which is the RT-PCR to detect the presence of one or more different sequences, is a reduction of times and/or costs to detect whether and which pathogen species is present in the sample. RT-PCR enables the discrimination among said pathogens with the help of one ore more primer couples (the couple being forward primer and reverse primer for a specific sequence), designed to amplify genes or specific sequences. With the presence of a TaqMan probe, the amplification signals the presence in the sample of extracted nucleic acid. If every nucleic acid sequence is uniquely specific for a single pathogen among those mentioned above, the RT-PCR enables the signalling of their presence.

In a preferred embodiment of said aspect of the invention, the nucleic acid sequences are DNA because RNA is labile and does not keep for a long time and Taq DNA polymerase works better with DNA. In a more preferred embodiment, DNA sequences are genes or specific sequences of the pathogens. Still more preferably said genes or specific sequences of the pathogens are ctr for N. meningitidis, P2 or Bex for H. influenzae and lyt or ply for S. pneumoniae.

In an even more preferred embodiment, the sequences to be used in said RT-PCR reaction are
SEQ ID NO. 1 and 2 as forward and reverse primer for N. meningitidis and SEQ ID NO. 91 as TaqMan probe sequence for N. meningitidis
SEQ ID NO. 3 and 4 or SEQ ID NO. 116 and 117 as forward and reverse primer for H. influenzae and SEQ ID NO. 92 or SEQ ID NO. 118, respectively as TaqMan probe sequence for H. influenzae
SEQ ID NO. 5 and 6 or SEQ ID NO. 94 and 95 as forward and reverse primer for S. pneumoniae and SEQ. ID NO. 93 or SEQ ID NO. 96, respectively as TaqMan probe sequence for S. pneumoniae

For Haemophilus influenzae use is also made of the P2 gene. The P2 gene, corresponding to one “Outer membrane protein” called P2, is common to all Haemophilus influenzae, both typable (i.e. capsulated) and non-typable (i.e. non capsulated or HINT). Accordingly, the use in the first phase of the test, the phase of detecting the germ in Realtime PCR (RT-PCR), of primers specific for the P2 gene enables detection of any Haemophilus, be it provided of a capsule or not. The test is thus very sensitive. This is particularly important nowadays because the capsulated Haemophilus which had been the most frequent in Italy, i.e. the b type, has been eliminated through mass vaccination in the paediatric age. Numerous cases of invasive bacterial infections due to HINT are emerging. For this reason, the search for HINT is even more necessary.

The RT-PCR reaction is carried out for n amplification cycles, preferably more than 40, even more preferably from 43 to 45 cycles, even more preferably 45 cycles. The reliability of the diagnostic outcome from this aspect of the invention is improved based on the number of RT-PCR cycles and 45 is the best value. An amplification of more than 45 cycles does not improve the reliability of the diagnostic outcome. The diagnostic outcome is compared with the threshold cycle (CT). The CT is the cycle wherein the fluorescence signals emitted are neatly measurable and statistically valid relative to the background noise. If at CT a significantly higher fluorescence and statistically meaningful is found, there may be determined, based on the frequency of the fluorescence emitted and on how the TaqMan fluorescent probes have been prepared in the TaqMan probes, which gene, and therefore which pathogen, is present in the sample and which is not. If no spontaneous fluorescence (i.e. different from the background noise) is measured, one may infer that the three genes specific for the three pathogens are not present in the DNA extracted from the sample taken.

According to said aspect of the invention, if the presence of said genes at the end of said RT-PCR reaction is detected or not detected, it may be concluded that said pathogen is or is not present in the sample taken from the host.

Embodiments of said aspect of the invention are reported in even greater detail in examples 1, 2 and 4. An advantage of the present method over the methods of the known art is that it allows the use of nucleic acid, preferably DNA extracted from a sample of biological tissue.

This happens because the primer sequences and the sequences used in the hydrolysis probes or Taqman probes are purposively selected so as to bind specifically to said gene sequences of the pathogens. Said selection of primer and probe sequences has the advantage, further to those mentioned above, to reduce, for S. pnewnoniae (through the use of primer for the lyt gene), the number of PCR cycles to arrive at CT cycle, thereby contributing to reduce to less than 4 hours the time to reporting of the sample taken, preferably to less than 3 hours and even more preferably to less than 2 hours per RT-PCR reaction. Indeed, without waiting for the end of the amplification cycles, a sample may already be considered positive and reported as such, when it appears to positive at the threshold cycle (CT), preferably by 28-35 cycles, even more preferably by 29-32 cycles.

The better sensitivity and independence from external influences (such as e.g. the presence of antibiotics or prolonged periods between the sampling and the reporting) of the method reduce the likelihood of false-negative results.

The selection of said specific genes is also advantageous because, since they are highly specific to said pathogen species, they do not display “cross-reactions” with other DNA sequences of other pathogens or of the host DNA and thereby reduce the likelihood of false-positive results. Selectivity is important because in the DNA extracted from the sample taken also present is the human host DNA and this has previously prevented performing said RT-PCR for said pathogens on samples taken from the host. Selectivity, further, minimizes prokaryote-prokaryote cross-reactions and thereby renders the diagnostic outcomes specific for the pathogen species that is present more reliable.

The reliability and rapidity of a result from a RT-PCR, as discussed above, are interconnected parameters that may be handled by the skilled in the art since they both depend on selectivity (strength and specificity) of the bond of the TaqMan primer and probes with the sequence of genes to be amplified. Said reliability and rapidity parameters depend on factors that include i) the pathogen load originally present and ii) strength and specificity of the binding of the TaqMan primer and probes with the sequence to be amplified. Since they are interconnected, the person skilled in the art may decide whether to use the signal at the CT to shorten the reporting time of the RT-PCR reaction (which he/she needs to, in an emergency situation) or to improve the reliability of the result (like e.g. when the original bacterial load was very low) and to wait for a report when the RT-PCR has arrived to more than 40 cycles, preferably 43 cycles or more preferably 45 cycles. Said aspect of the invention of the RT-PCR reaction may be repeated to detect the presence of each species separately or, advantageously, the RT-PCR may be carried out at the same time for two of the pathogens or for all the pathogens at once. The advantage of carrying out the RT-PCR for all the pathogens at once is the decrease of the reporting time from the DNA extraction from the sample to less than 4 hours, preferably to less than 3 hours or even more preferably less than 2 hours.

In order to further improve the reliability of the RT-PCR method, it is preferable to introduce and intermediate step between the extraction of the nucleic acid and the amplification of the nucleic acid by RT-PCR. In said intermediate step the nucleic acid solution is incubated with a DNA-ase specific for sequences containing dUTP, such as e.g. Amperase UNG. Said step is particularly useful when several RT-PCR or PCR phases and DNA extractions are performed in the same laboratory. Said method works only if to amplify DNA by PCR in the same laboratory dUTP is used instead of dTTP. The exchange of deoxy-nucleotides enables the elimination of the possibility of contaminations from sequences amplified by PCR which could be present in the laboratory and in the equipment prior to the RT-PCR reaction and therefore the elimination of the possibility that these give a false-positive result in the subsequent PCR reaction. Accordingly, it is preferable to always use dUTP instead of dTTP in the PCR reactions of the invention.

In another aspect of the invention, the precise serogroup and/or serotype of a pathogen may be identified if the pathogen species that is present is already known. This method may follow identification of the species with the RT-PCR reaction as described above or it may be carried out in a sequence of separate steps wherein the DNA is extracted from the biological sample and then the serogroup is identified.

In order to identify the serogroup according to the invention, one or more PCR reactions need to be carried out.

The primers used in the reaction depend on the species upon which one wants to identify the serogroup and on the gene giving specificity to the serogroup. The primers determine which DNA sequence is amplified and the amplification of said DNA sequence signals which specific gene serogroup of the species is present.

In some serogroups, a distinction may be made among several serotypes. Serogroups are denominated in the art with numbers for some specific genes (see Tables 1-3 and 7) and serotypes with the number and a subsequent letter. In said aspect of the invention, the serogroups and/or serotypes to be detected are limited to those belonging to the pathogens described above and known as infective.

In said aspect of the invention serogroups and/or serotypes of the pathogens are detected according to the invention in one or two PCR phases in which there is one ore more PCR reactions in parallel. The at least one PCR reaction may be carried out with all the apparatuses or reagents known in the art for said type of reaction.

It is preferable to use dUTP instead of dTTP for the reasons described above.

It is preferable to determine the product amplified by the PCR reaction by agarose gel electrophoresis and to dye with ethidium bromide, because in some embodiments of said aspect of the invention it is necessary to use a calibration of the measurement of the amplified sequences to determine the serotype and/or serogroup.

It is also preferable to introduce in the at least one PCR reaction at least a couple of primers specific for positions in gene sequences known as common sequences for the whole of said species. In the context of the present inventions, said couples of primers are called control primers. The amplification of sequences from said control primers shows two things:
that the PCR reaction has occurred,
and that if there has been no other positive amplification result for all other couples of primers, the nucleic acid sample, preferably DNA, comprises that of a prokaryote belonging to said species but with a serotype not recognizable by the method.

Some of said unrecognizable serotypes are not recognizable because the primer sequences are not available, and this may be demonstrated through a subsequent microbiologic culture method. Alternatively, the absence of a reaction for primers of serotypes and/or precise serogroups means the detection of a new serotype or serogroup, which must however be verified subsequently through culture methods.

For N. meningitidis, said couple of control primers are SEQ ID NO. 1 and 2 and/or 7 and 8, for H. Influenzae are SEQ ID NO. 21 and 22 and for S. pneumoniae they are SEQ ID NO. 29 and 30.

In a preferred embodiment of said aspect of the invention, said at least one PCR reaction is carried out in a buffer solution containing betaine, such as e.g. buffer solution Q from Qiagen, Hilden, Germania, at a concentration varying from 0.7 to 1.3, preferably from 0.85 to 1.15 and even more preferably at concentration from 0.98 to 1.02 M. The buffer solution comprising betaine at said concentrations enables amplification with greater precision of the templates rich in GC and further prevents that the Taq polymerase of PCR get off the DNA during amplification. Said effects enable maximisation of the difference between the expression from PCR sequences and background noise due to the massive presence of host DNA in the sample.

It is further preferable that all primers used in the at least one PCR reaction be at the same concentration. Said concentration of the primer may be between 0.15 and 0.25 mM, preferably 0.2 mM.

If N. meningitidis is present in the sample, the serogroup of the pathogen is preferably determined with a single PCR reaction because there is the possibility of cross-reactions. Said reactions may be split into one or more simultaneous phases, wherein every single PCR reaction is carried out in parallel. In each simultaneous phase of PCR reactions, at least a couple of control primers is needed. The reagents for every PCR reaction comprise primers specific for a different serogroup of N. meningitidis known in the art to be infective and are selected, excepting from the couples of control primers, from the SiaD gene. Said primers to be used in the reaction are here listed in Table 1:

TABLE 1
List of primers used in the serotyping of N. meningitidis

| | Forward | Reverse | Gene | | Weight of the |
|---|---|---|---|---|---|
| | primer | primer | specific | Serogroup of | resulting |
| | SEQ ID | SEQ ID | for the | N. meningitidis | amplificate |
| | NO. | NO. | primers | indicated | (bp) |
| Couple of | 1 | 2 | CtrA | All | 111 |
| control | | | | | |
| primers I | | | | | |
| Couple of | 7 | 8 | Isll06 | All | 331 |
| control | | | | | |
| primers II | | | | | |
| Couple of | 9 | 10 | SiaD | B | 457 |
| primers | | | | | |
| Couple of | 11 | 12 | SiaD | C | 442 |
| primers | | | | | |
| Couple of | 19 | 20 | SiaD | A | 400 |
| primers | | | | | |
| Couple of | 13 | 14 | SiaD | YoW135 | 698 |
| primers | | | | | |
| Couple of | 15 | 16 | SiaD | W | 120 |
| primers | | | | | |
| Couple of | 17 | 18 | SiaD | Y | 120 |
| primers | | | | | |

The results of amplified sequences produced by each phase of PCR reactions in parallel are preferably identified by a method of runs on agarose gel. Each single reaction is placed on a separate lane.

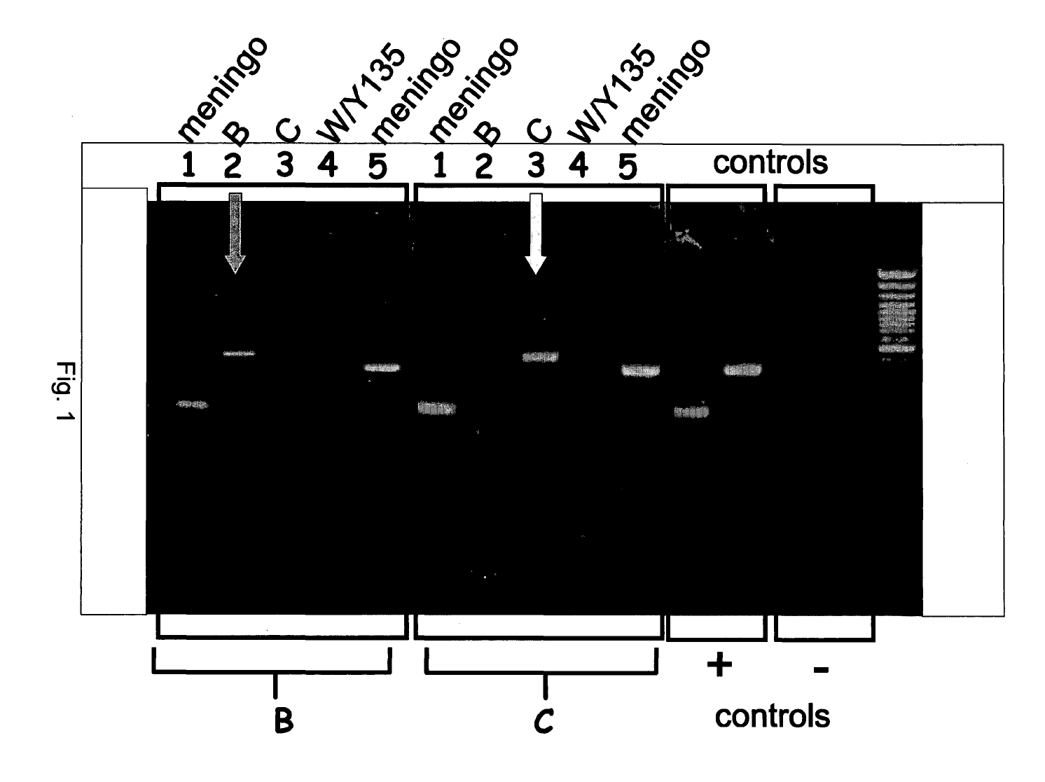

FIG. 1 is an agarose gel showing the results of PCR reactions to detect the serogroup of N. meningitidis in 2 samples. It may be seen from the presence of bands in separate lanes as in sample 1 that the serogroup of N. meningitidis is SiaD B and in sample 2 is serogroup SiaD C, and how in both samples serogroup SiaD Y/W135 is not present.

In each gel resulting from a phase, there is at least one lane wherein the amplificate deriving from the control primers present may be seen. The presence of an amplificate in another lane (excepting that of controls) signals the presence of that serotype to which the lane and the primers used correspond. In FIG. 1, lane 1 shows the expression from the couple of control primers I and lane 5 shows the expression of primer from the couple of control primers II.

The choice whether to use one or two simultaneous phases, and which primer to select depends on the requirements of the diagnostic test and the person skilled in the art is able to manage the method so as to satisfy said needs.

In a preferred embodiment, reactions are split into two phases, since the SiaD W and Y serogroups are poorly present (nearly 95% of the cases of infection with N. meningitidis in Italy are from the SiaD B and C type). It is preferable, for advantages of convenience and costs, to use the couple of primers SEQ ID NO. 12 and 13 in a first phase of PCR reactions, and, if the nucleic acid present in the sample is amplified with said couple of primers SEQ ID NO 12 and 13, to continue with a second phase of PCR reactions with at least one group of control primers and SEQ ID NO 14-15 and 16-17 to determine the precise serogroup.

For N. meningitidis, it may be inferred whether and which serogroup is present through the presence of the bands in the special lanes, but the molecular weight of the bands resulting may also indicate which serogroup is present, by referring to the values listed in Table 1. The molecular weight may be inferred on the agarose gel with any method known in the art, preferably calibrating with a scale of bands wherein each band in the scale represents an increase of 100 bp (bp, in the context of the present invention means base-pairs). In the context of the present invention, the numbers given for by are to be interpreted with the precision that the person skilled in the art would use, especially when interpreting the measurement of the amplificate on lanes of agarose gel. All the products for the calibration of molecular weights on agarose gel with a scale of 100 by are valid in the phase (iii) of the present invention.

If one wants to determine the serogroup of H. influenzae, the PCR reaction may be carried out in one or more PCR reactions, and, if multiple reactions are carried out, they may be carried out in parallel or in separate time phases. The at least one PCR reaction is carried out with methods known in the art and the primers to be used are those listed in Table 2.

TABLE 2
List of primers used in the serotyping of H. influenzae

| | | | | Weight of | |
|---|---|---|---|---|---|
| | Forward | Reverse | Gene | the | |
| | primer | primer | specific | resulting | Serogroup of |
| | SEQ ID | SEQ | for the | amplificate | H. influenzae |
| | NO | ID NO | primers | (bp) | indicated |
| Couple of | 21 | 22 | P6 | 198 | All |
| control | | | | | |
| primers | | | | | |
| Couple of | 23 | 24 | TEM | 458 | beta-lactamase |
| control | | | | | producers |
| primers | | | | | |
| Couple | 25 | 26 | BexA | 343 | Capsulated |
| of | | | | | Haemophilus |
| primers | | | | | influenzae |
| | | | | | (from a to f) |
| Couple | 27 | 28 | Cps | 224 | Only B type |
| of | | | | | capsulated |
| primers | | | | | Haemophilus |
| | | | | | influenzae |

The serogroup present in the sample is determined by means of the bands present on the agarose gel following electrophoretic runs as described above for N. meningitidis. The preferred embodiment is in a single reaction wherein all the primers for the different serogroups are present, because the risk of cross-reactions between the different serogroups is absent. The advantage is the elimination of costs and of time necessary to carry out a plurality of PCR reactions to determine the serogroup of H. influenzae. In said method it is necessary to use the band molecular weight calibration method, with reference to the values given in Table 2, to determine if and which serogroup is present in the sample, as described above for N. meningitidis.

If S. pneumoniae is present in the sample, the serogroup and/or serotype from the 90 serotypes known in the art has to be determined. In order to avoid cross-reactions it is possible that a plurality of PCR reactions are needed to determine the specific serogroup. Primers specific to the locus of the cps gene are used. The couple of control primers are also specific to the cps gene. The PCR reactions are carried out with groups of Mix-PCR, which comprise the sequence of control primers and a plurality of groups of primer sequences. The amplification of sequences is measured, as described above with an electrophoretic run on agarose gel for each PCR reaction.

When a serogroup is present in the nucleic acid extract upon which the at least PCR reaction is carried out, the electrophoresis results display two bands on the gel instead of one, because one band is always due to the control primers. Since the PCR reactions contain a plurality of couples of PCR in groups denominated Mix-PCR, the calibration system with the molecular weight of the amplificate (ref. Table 3) is used, as described above for N. meningitidis.

The primer sequences to be used are listed in couples in Table 3:

TABLE 3
List of primers used in the serotyping of S. pneumonite (all the primers
specific for the cps gene, based on the precise serogroup and/or serotype)

| | | | | Weight of |
|---|---|---|---|---|
| | Forward | Reverse | Indicated S. | the resulting |
| | primer SEQ | primer SEQ | pneumoniae | amplificate |
| | IDNO | IDNO | serogroup | (bp) |
| Control | 29 | 30 | All | 160 |
| primer couple | | | | |
| Primer couple | 31 | 32 | 19F | 130 |
| Primer couple | 33 | 34 | 22F | 643 |
| Primer couple | 35 | 36 | 3 | 371 |
| Primer couple, | 37 | 38 | 6A/B | 220 |
| Primer couple | 39 | 40 | 19A | 478 |
| Primer couple | 41 | 42 | 9V | 507 |
| Primer couple | 43 | 44 | 4 | 430 |
| Primer couple | 45 | 46 | 14 | 268 |
| Primer couple | 47 | 48 | 12 A/B/F | 376 |
| Primer couple | 49 | 50 | 7F/A | 826 |
| Primer couple | 51 | 52 | 11 A/D/F | 463 |
| Primer couple | 53 | 54 | 33F/A | 338 |
| Primer couple | 55 | 56 | 16F | 988 |
| Primer couple | 57 | 58 | 35 B | 677 |
| Primer couple | 59 | 60 | 18A/B/C/F | 573 |
| Primer couple | 61 | 62 | 38 F | 574 |
| Primer couple | 63 | 64 | 31 | 701 |
| Primer couple | 65 | 66 | 15B/C | 496 |
| Primer couple | 67 | 68 | 8 | 294 |
| Primer couple | 69 | 70 | 10A | 628 |
| Primer couple | 71 | 72 | 35 F | 517 |
| Primer couple | 73 | 74 | 34 | 408 |
| Primer couple | 75 | 76 | 1 | 280 |
| Primer couple | 77 | 78 | 17F | 693 |
| Primer couple | 79 | 80 | 20 | 514 |
| Primer couple | 81 | 82 | 15 A | 436 |
| Primer couple | 83 | 84 | 7C/B | 260 |
| Primer couple | 85 | 86 | 18C/A/B/F | 354 |
| Primer couple | 87 | 88 | 5 | 362 |
| Primer couple | 89 | 90 | 23 F | 177 |

Primers 59/60 and 85/86 relate to serotype 18. Primers 59/60, amplifying, give a band of 573 by and the 85/86 primers give one of 354 bp. It is important to maintain both because, when they are placed inside their respective mixes, the length that they have allows one to distinguish them from other serotypes contained in the mix.

The sequences are preferably subdivided in the following 9 groups, to carry out 9 separate PCR reactions:

Mix-PCR 1: SEQ ID NO. 29-30, 31-32, 37-38, 41-42, 43-44, 45-46, 87-88 and 91-92;

Mix-PCR 2: SEQ ID NO. 29-30, 33-34, 35-36, 37-38 and 39-40;

Mix-PCR 3: SEQ ID NO. 29-30, 41-42, 43-44, 45-46 and 47-48;

Mix-PCR 4: SEQ ID NO. 29-30, 49-50, 51-52 and 53-54;

Mix-PCR 5: SEQ ID NO. 29-30, 55-56, 57-58, 59-60 and 61-62;

Mix-PCR 6: SEQ ID NO. 29-30, 63-64, 65-66, 67-68 and 69-70;

Mix-PCR 7: SEQ ID NO. 29-30, 71-72, 73-74, 75-76 and 77-78;

Mix-PCR 8: SEQ ID NO. 29-30, 79-80, 81-82, 83-84 and 85-86;

Mix-PCR 9: SEQ ID NO. 29-30, 87-88, 89-90 and 91-92.

An advantage of using said Mix-PCR couple of primers is that the cross-reaction is minimized at a value between 0 and 0.2% of the cases, even more preferably between 0 to 0.1% of the cases.

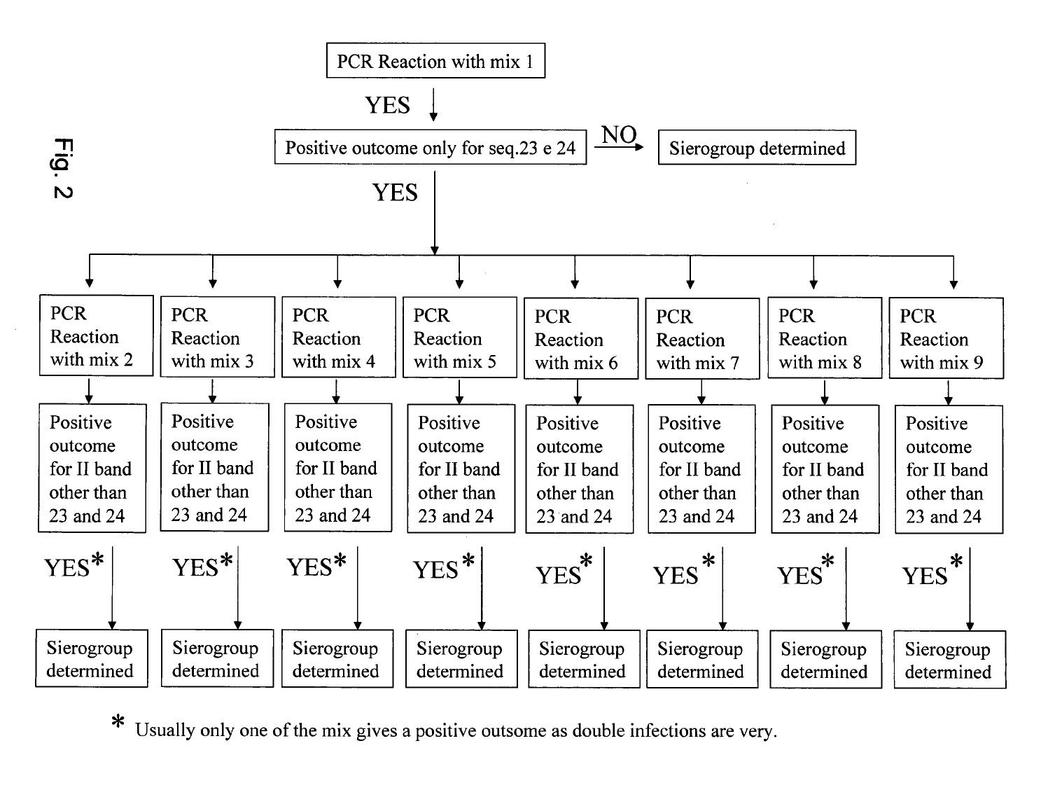

In an even more preferred embodiment, the Mix-PCR groups are made to react in two phases of PCR reactions as represented in FIG. 2.

An advantage of the use of said chronological order of PCR reactions with said Mix-PCR groups 1-9 is that it allows the serogroup and/or the specific serotype to be identified as quickly as possible based on the likelihood to find specific serogroups, preferably in less than 3 hours, even more preferably in less than 2 hours, because the primers present in the Mix-PCR 1 are specific for the primers present in over 80%, between 80% and 90% of the cases reported in Italy.

In a preferred embodiment, there may be introduced between extraction and amplification a step wherein the solution of nucleic acid is incubated with DNA-ase specific for sequences containing dUTP, as e.g. Amperase UNG. The advantage of introducing said step is to improve the reliability of the result, as mentioned above. The same considerations apply to said preferred embodiment and therefore the PCR reactions preferably comprise the use of dUTP instead of dTTP.

A preferred embodiment of the whole method of the invention is a method wherein:
DNA is extracted from the sample;
One or more than one RT-PCR reactions are carried out to detect the presence or absence of at least one of the pathogens in a first portion of the DNA extracted from the sample, preferably all three of them at the same time;
One or more than one PCR reactions are carried out to determine the serogroup of at least one of the pathogens in a second portion of the DNA extracted from the sample, knowing already which species are present in the sample thanks to the RT-PCR reaction.

Said preferred embodiment may incorporate all the steps and reagents as described above. The advantage of said embodiment is that it allows one to know, in a period lower than 8 hours, preferably than 6 hours, more preferably than 5 hours and even more preferably less than 4 hours, if one or more pathogens are present in a sample of a biological tissue and their serogroup and/or serotype, together with all the advantages mentioned above for said aspects of the invention, such as for example the reliability and versatility of the method.

Another advantage of the methods according to the invention is that they nearly always use equipment designed for carrying out reactions automatically, reducing the likelihood of human mistakes in the operation and consequently improving statistically the efficiency of the system, especially when it is compared with methods known in the art as the diagnosis with culture techniques.

Many of these advantages derive from the selection of specific primer sequences, thus it is another object of the invention primer sequences comprised in the group of : SEQ ID 1 and SEQ ID 2; SEQ ID 116 and SEQ ID 117; SEQ ID 3 and SEQ ID 4; SEQ ID 5 and SEQ ID 6; SEQ ID 94 and SEQ ID 95; SEQ ID 99 and SEQ ID 100; and the TaqMan probes SEQ ID NO. 91-92-92-93-96-118-101-104-107-110-113.

Diagnostic kits are another aspect of the invention. In one embodiment, the diagnostic kit is for detecting in the DNA extracted from the sample taken from a human being the presence of a pathogen selected from the group consisting of N. meningitidis, H. influenzae and S. pneumoniae or Adenovirus. Said kit optionally comprises a first compartment comprising reagents to extract the DNA from said sample, among which preferably protease K; and a second compartment comprising reagents to carry out RT-PCR, said reagents comprising SEQ ID 1 and SEQ ID 2; SEQ ID 116 and SEQ ID 117; SEQ ID 3 and SEQ ID 4; SEQ ID 5 and SEQ ID 6; SEQ ID 94 and SEQ ID 95; SEQ ID 99 and

SEQ ID 100; and the TaqMan probes SEQ ID NO. 91-92-92-93-96-118-101-104-107-110-113.

In a preferred embodiment of the kits, the buffer solution containing betaine at concentrations from 0.7 to 1.3 M is present as a reagent for the PCR reactions of the third compartment.

### EXAMPLE 1

### Study of the Incidence of S. pneumoniae in the Paediatric Population

A clinical study was carried out on children aged between 0 and 14, wherein a suspicious case with an infection by S. pneumoniae is classified if it has clinical symptoms indicating an infection by S. pneumoniae (symptoms of pathologies such as meningitis, sepsis, osteomyelitis, arthritis or pneumonia). An analysis was carried out with a method according to the invention and it was compared with an analysis using methods of classical analysis by means of cultures. Blood was taken from the patients and, in the suspicious cases of meningitis samples of CSF were also taken. Samples were taken for analyses with microbiological methods and the method according to the invention.

### Method According to the Invention

### i) DNA Extraction

Genomic prokaryote DNA was extracted from the biological samples using QIAmpDNA mini kit (Qiagen, Hilden, Germany) following the protocols suggested by the kit producer from 200 micro-litres of biological liquid.

### ii) RT-PCR

In 25 micro-litres reagents for RT-PCR were prepared, which contained:
2× TaqMan Universal Master Mix (Applied Biosystem, Foster City, Calif., USA)
SEQ ID NO. 1-2 and 5-6 primers at concentrations of 300 nM,
TaqMan probe sequences SEQ ID No. 93 and 95 at concentrations of 50 nM,
6 micro-litres of extracted DNA.

Reactions were carried out in triplicate.

A negative control and a positive control for pathogens were included in every reaction, wherein the positive control used a sample containing S.pneumoniae and the negative control was a sample not containing S. pneumoniae (usually water).

DNA was amplified with the following parameters:

50° C. for 2 minutes for UNG digestion with Amperase, 95° C. for 10 minutes followed by 45 cycles, wherein every cycle is divided in two phases: 95° C. for 15 sec. and 60° C. for 1 minute.

iii) PCR Reactions

Several PCR reactions were subsequently carried out on the DNA extracted from the samples according to step (i).

32 couples of primers according to table 3 (SEQ ID No. 29-92) were used in 9 multiplex reactions, with the chronological order displayed in FIG. 2.

PCR reactions were carried out in 25-microliter solutions consisting of:
1× PCR buffer (Applera, Applied Biosystem, Foster City, Calif., USA), containing Q Qiagen solution
200 microM of each dNTP, (New England Biolabs, Beverly, Mass., USA),
2,5 mM MgCl₂;
2 units of AmpliTaq Gold DNA polymerase (Applied Biosystem, Foster City, Calif., USA),
0.2 mM of each primer and
5 micro-litres of DNA extract.

The PCR reaction was carried out in a Perkin-Elmer GeneAmp PCR System 9600 (Applied Biosystem, Foster City, Calif., USA) with the following parameters:
94° C. for 15 minutes,
Followed by 35 amplification cycles at 94° C. for 30 seconds,
54° C. for 90 seconds and
72° C. for 60 seconds.

The products of the PCR reactions were analyzed by electrophoresis on agarose gel 2% Nusieve (Cambrex Bio Science Inc., Rockland, Me.) with 1× TAE buffer. Gels were dyed with ethidium bromide (0.5 micrograms/ml) and their images were recorded. The length of the PCR products was determined by a calibration of the bands with a scale of bands which subdivides every 100 bp.

Microbiological Method with Cultures for Comparison

Cultures with blood were grown by using samples taken with the bottle system BACTEC TM PLUS (Necton Dickinson and Co., Sparks, Md., USA). The sample was grown in agar-blood and the presence of pneumococci was assessed by susceptibility to optochin and bile-solubility according to standard methodologies (see: Arbique J C, Poyart C, Trieu-Cuot P, Quesne G, Carvalho Mda G, Steigerwalt A G, Morey R E, Jackson D, Davidson R J, Facklam R R. Accuracy of phenotypic and genotypic testing for identification of Streptococcus pneumoniae and description of Streptococcus pseudopneumoniae sp. nov. J Clin Microbiol. 2004;42(10):4686-96.

### Results

92 patients were enrolled in the study with clinical symptoms indicating an infection with S. pneumoniae. Table 4 (patients were divided in a way different than the previous one) shows the results of each patient:

TABLE 4
Results of PCR and cultures for S. pneumoniae in 92 patients

| | Patients | | | Culture | |
|---|---|---|---|---|---|
| | (n = 92) | PCR results | | results | |
| Arthritis | 4 | Positive | 2 | Positive | 0 |
| | | Negative | 2 | Negative | 4* |
| Pneumonia | 80 | Positive | 16 | Positive | 1 |
| | | Negative | 64 | Negative | 79 |
| Meningitis/sepsis | 8 | Positive | 4 | Positive | 1 |
| | | Negative | 4# | Negative | 7§ |

*1 was positive for Staphylococcus aureus
#4 were positive for Neisseria meningitidis
§1 was positive for Neisseria meningitidis

All the patients diagnosed as non-infected according to the method according to the invention were proved to be non-infected also with the culture method known in the art.

On the contrary, 22 patients were diagnosed as infected by S. pneumoniae with the method according to the invention and only 4 with microbiological culture methods known in the art. Therefore, the PCR method proved to be more effective in diagnosing infections by S. pneumoniae with respect to the microbiological culture method for 81.8% ((22-4)/22) of the cases.

Out of 22 patients diagnosed as infected by RT-PCR, 7 had previously had an antibiotic therapy. All 7 were not considered to be negative with the culture method. 15 had not done antibiotic therapy and, out of these, 4 (4/15=26.7%) had been detected to be infected by the culture method.

This examples shows the versatility (in showing the presence of N. meningitidis beside S. pneumoniae), the greater sensitivity and reliability of the method (15 patients diagnosed vs. 4 with methods known in the art) and the overcoming of the problem raised by an antibiotic therapy set at the same time of, or prior to, the diagnose. 19 out of 22 cases were typed by the molecular method (in 3 patients the bacterial load was too low and therefore the bacterial DNA too scant to perform the typing). The distribution in the various serotypes/serogroups is shown in Table 5. Said information, having medical relevance, concerning the population of specific serogroups and/or serotypes specific would not have been obtainable without the method according to the invention.

TABLE 5
Distribution in the various serotypes/serogroups in
22 cases of invasive infection with S. pneumoniae

| | Patients | |
|---|---|---|
| | (n = 22) | Pneumococcus Serotype |
| Arthritis | 2 | NT |
| | | 22F |
| Pneumonia | 16 | 4 |
| | | 6A/B |
| | | 6A/B |
| | | 6A/B |
| | | 8 |
| | | 8 |
| | | NT |
| | | 14 |
| | | 18C |
| | | 18C |
| | | NT |
| | | 1 |
| | | 19F |
| | | 3 |
| | | 4 |
| | | 23F |
| Meningitis/sepsis | 4 | 4 |
| | | 6A |
| | | 7B |
| | | 23F |
| | | 9V |

NT = non typed

### EXAMPLE 2

### Comparison of the Sensitivity of the Genes Chosen for the RT-PCR

By way of example, a comparison was made as concerns the efficiency of detection of the presence of S. pneumoniae according to the RT-PCR between the lyt gene and another gene known in the art and capable of detecting the presence of S. pneumoniae through

PCR: the ply gene.

As a primer for the RT-PCR directed towards lyt SEQ ID No. 5 and 6 were used and SEQ ID No. 89 was used as a TaqMan probe couple.

As a primer for the RT-PCR directed towards ply SEQ ID No. 90 and 91 were used and SEQ ID No. 91 was used as a TaqMan probe couple.

A RT-PCR method was performer on 8 samples of DNA extracted according to Example 1 (i) and e reacted according to the method in Example 1 (ii), wherein the cycles were measured at which the positivity appeared, the test has been protracted for a total amplification of 45 cycles. The results are reported in table 6.

TABLE 6
Number of cycles in a RT-PCR reaction with TaqMan primer
and probe sequences directed to obtain a fluorescence
signal in the presence of lyt or ply genes

| | No. of cycles if RT-PCR | No. of cycles if RT-PCR |
|---|---|---|
| | is directed to lyt gene | is directed to ply gene |
| Sample | amplification | amplification |
| 1 (blood) | 29 | 31 |
| 2 (blood) | 34 | 35 |
| 3 (blood) | 40 | Negative |
| 4 (blood) | 25 | 25 |
| 5 (blood) | 31 | 33 |
| 6 (CSF) | 24 | 25 |
| 7 (CSF) | 32 | 34 |
| 8 (diluted CSF) | 38 | 39 |
| Average of the 7 | 30.4 | 31.7 |
| samples (sample 3 | | |
| excluded) | | |

From the results in table 6, it may be seen that a method of scanning by RT-PCR for S. pneumoniae, TaqMan primers and probe sequences are more sensitive towards the lyt gene thank towards the ply gene. The average shows a difference of a cycle, but it must be remembered that said cycles grow exponentially, and thus the difference in sensitivity between the thirtieth cycle and the thirty-first cycle is remarkable to demonstrate the difference in sensitivity between the genes. Further, with the ply gene sample No. 3 appeared to be negative (which would correspond to a “not infected by S. pneumoniae” report). The lyt gene has instead allowed detection of the infection by S. pneumoniae, even though this was present with a low bacterial load.

### EXAMPLE 3

### Demonstration of the Efficiency in the Serotyping of Pathogens

Strains of S. pneumoniae of serotype/serogroup 6B, 9V, 14, 19A and 23F were obtained from ATCC. In order to verify the specificity of the investigation, strains of S. pneumoniae belonging to the serogroups/serotypes listed above were obtained from ATCC. All were found to be positive in the respective mix and negative in all the mixes mix wherein the specific primers were not present.

Serotyping was made with the same protocol. The starting sample was a pellet of centrifuged bacteria subsequently re-suspended in a buffered saline solution (PBS). The thus resulting sample was treated with the same protocol of the human biological fluids reported in Example 1(i) and (iii).

The results were compared with the serotype already established for said strains and in all the cases the serogroup determined by the multiplex PCR method has been found to be equal to that stated in the ATCC catalogue, showing a 100% accuracy of the method in determining the precise serotype of a pathogen.

### EXAMPLE 4

### Method Effect Demonstration on N. meningitidis

Over a year there have been diagnosed, by RT-PCR as reported in Example 1 (i) and (ii), 6 cases of meningitis due to an infection, of those only 2 were found to be diagnosable through culture. RT-PCR based methods have proved to be three times more sensitive than the culture based methods.

As for S. pneumoniae, also for Neisseria meningitidis in all meningitis cases a serotyping has been made by PCR, using a protocol similar to those explained above in Example 1 (iii) and using the primers listed in Table 1. The serotyping is possible, with the culture based methods only when the cultures are positive, in our example in 2 cases out of 6. In conclusion, 2 of the 3 cases of meningitis would not have been diagnosed and, as a consequence, not even typed with the method known in the art and instead present in the present invention.

### EXAMPLE 5

### Method for Differentiating Serotypes 6A and 6B of Streptococcus pneumoniae

Streptococcus pneumoniae (pneumococcus) is a gram-positive germ of which about 90 serotypes are known. Only some of these seem to be responsible for grave invasive bacterial diseases. A vaccine is available commercially, which is suitable for use with children, and which contains only 7 of the 90 serotypes, the most frequent and most dangerous for health. Such serotypes (4,6B, 9V,14,18C,19F,23F) alone are responsible for 80% of the invasive bacterial forms. It is thought, however, that through the years, both due to the immunologic pressure induced by the vaccine and to a spontaneous shift between serotypes caused by the phenomenon called “secular trend”, the strains currently most frequent are being substituted by different strains. In particular it seems that serotypes 6A and 19A are replacing the previously most frequent strains. serotype 6A is not present in the vaccine currently commercially available but it is present in two vaccines close to being produced, the 10-valent and the 13-valent. Then, identifying serotypes 6A and 6B and knowing the distribution over the territory is very important to be able to under stand whether the current vaccine maintains its efficiency or it has to be substituted by new vaccines.

### Method

All biological samples (blood, liquor, nasal-pharyngeal tampons, pleuric liquids, bacterial isolates etc.) that were found to be positive by PCR real time for Streptococcus pneumoniae and that were subsequently found to be positive by PCR multiplex for serotype 6 are amplified by PCR using the primers (wciP gene) and the reaction mixture described in the following:

Primers
Primer forward AATTTGTATTTTATTCATGCCTATATCTGG
Primer reverse TTAGCGGAGATAATTTAAAATGATGACTA

Reaction mix

| | | |
|---|---|---|
| 10 x Qiagen | 2.5 μl | |
| dNTPs | 2.0 μl | (2.5 mM) |
| Primer for | 1.0 μl | (10 pmol/μl) |
| Primer rev | 1.0 μl | (10 pmol/μl) |
| Taq Qiagen | 0.2 μl | |
| H₂O | 13.3 μl | |

5 μl of extracted DNA

### Amplification Protocol

The PCR reaction is carried out in a Perkin-Elmer GeneAmp PCR system 9600 (Applied Biosystem, Foster City, Calif., USA) with the following parameters:

1. 95° C. for 15 minutes,

2. Followed by 30 amplification cycles at
94° C. for 30 seconds,
55° C. for 45 seconds and
72° C. for 60 seconds.

3. 72° C. for 10 minutes

These primers amplify both serotype 6A and serotype 6B and yield a product of 250 pairs of bases within which 2 polymorphic sites (base 118 e 128 Table 7) exist, i.e. two sites that are different between 6A and 6B.

TABLE 7
sequence amplified according to the method described above
(underlined are the positions of the primers;
in bold font are the polymorphic sites)

| | Amplified sequence (bases 1-250) |
|---|---|
| 6A | 1 aatttgtattttattcatgcctatatctgggggtgtactgcaggttttaatcatgcattg |
| | 61 ctagagatggttccttcagttgatattgataaagattatttatatatagaaaaactggct |
| | 121 catgatagttattttgcaaagtttgcactagagtatgggaaggtgttgttctgccctgag |
| | 181 caactggtcttgtatcgaagacatggacataatgtaacaactagtcatcattttaaatta |
| | 241 tctccgctaa |
| 6B | 1 aatttgtattttattcatgcctatatctgggggtgtactgcaggttttaatcatgcattg |
| | 61 ctagagatggttccttcagttgatattgataaagattatttatatatagaaaaactgtct |
| | 121 catgataattattttgcaaagtttgcactagagtatgggaaggtgttgttctgccctgag |
| | 181 caactggtcttgtatcgaagacatggacataatgtaacaactagtcatcattttaaatta |
| | 241 tctccgctaa |
| | Amplified sequence (bases 116-140, polymorphism seat) |
| 6A | . . . tgg ctc atg ata gtt att ttg caa a . . . |
| 6B | . . . tgt ctc atg ata att at ttg caa a. . . |
| | 118 128 |

The polymorphism present on base 118 of serotype 6A is recognized as a cleavage site by the restriction enzyme CviKI-1 which leaves, instead, codon 118 of serotype 6B undigested.

In order to distinguish 6A from 6B, therefore, it is necessary to perform an incubation step (=digestion) of the amplificate with enzyme CviKI-1 which will cleave serotype 6A in two fragments, whereas it will leave serotype 6B undigested (hence intact).

Digestion Reaction with CviKI-1 (New England BioLabs)

| | |
|---|---|
| CviKI-1 enzyme | 0.2 μl |
| 10x (NEB 4) | 2 μl |
| H₂O | 2.8 μl |

15 μl of PCR reaction product.

The mixture is kept at 37° C. for 1 h; at the end of the incubation 10 μl of it are run over 3% agarose gel.

In the case of serotype 6A the restriction enzyme produces a cleavage at codon 118 and therefore 2 bands of different size will be highlighted: 118 pb and 132 pb.

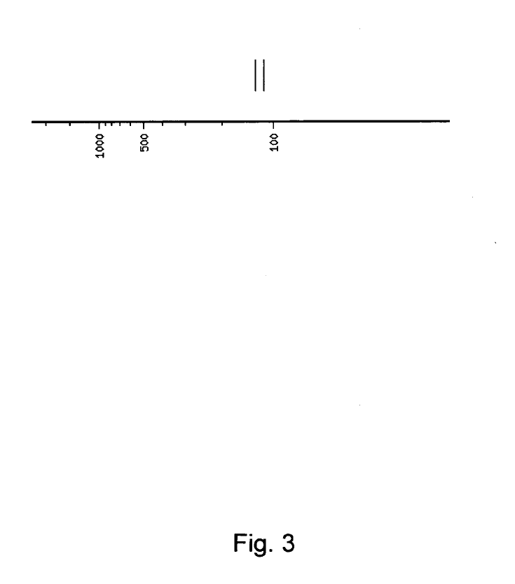

FIG. 3—Example of 3% Gel of serotype 6A after digestion (serotypes 6A identified with accession number AF246898, AY078347, AY078344, AY078343, AY078342, CR931638). In the case of serotype 6B the restriction enzyme does not produce any cleavage.

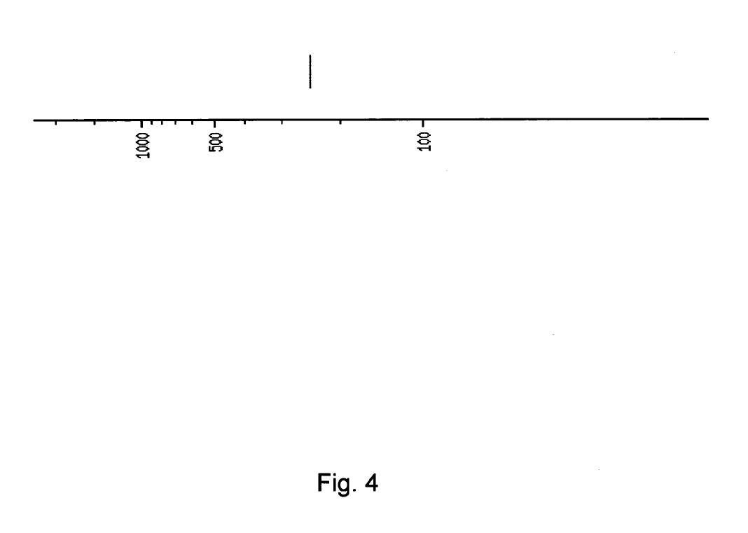

FIG. 4—Example of 3% Gel of serotype 6B (accession number: AY078341, AY078340, AY078339, CR931639, AF316640, AF298581). In some 6B serotypes (identified with accession number AY078346, AY078345 e AF246897) a cleavage will occur at codon 44 and therefore 2 bands of different size will be highlighted: 44 pb and 206 pb (FIG. 3). These will however be perfectly distinguishable based on the fragmentation pattern of 6A seen in FIG. 3 due to the size of the amplificate.

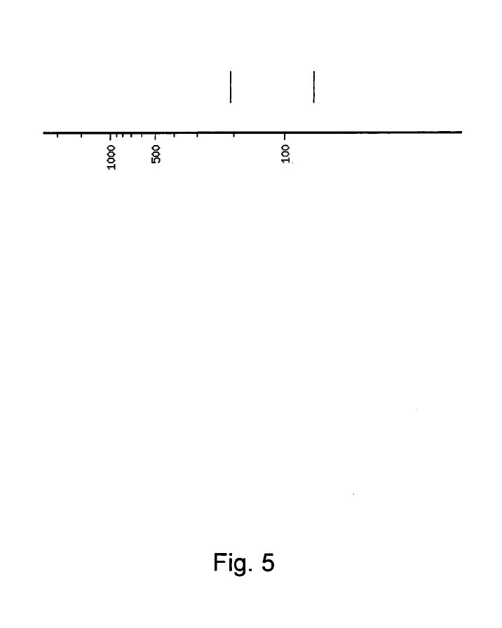

FIG. 5—Example of 3% Gel of serotype 6B AY078341, AY078340, AY078339, CR931639, AF316640, AF298581

### Sensitivity

The method was tested on strains of serotypes 6A and 6B purchased from ATCC and on strains of Streptococcus pneumoniae grown in culture and identified by standard serologic methods (Pneumotest kit, Biogenetics, Pone San Nicolò, Padova, Italia), for a total of 7 strains 6A and 7 strains 6B. The test gave a positive result (correct identification) in all the “isolates”, managing to identify the serotype in all of the cases (14/14, 100%).

### Specificity

The method was tested on germs other than Streptococcus pneumoniae (15 samples positivs for different germs) and on Streptococcus pneumoniae of serotype other than 6 (18 samples). In none of the cases with the primers described above amplification occurred, thereby showing a 100% specificity.

### EXAMPLE 6

### Method for Identifying Germs in Newborn Children

### Background—Usefulness of the Method

Molecular biology methods (Realtime PCR and standard PCR) have shown a significantly greater sensitivity equal specificity with respect to culture methods in the microbiologic diagnostics of invasive bacterial infections.

The germs most often causing infections in small children (from 0 to 3 months of age) are not the same causing a pathology in older children.

For this reason it is useful to create a different panel, containing specific primers and probes (Table 8) for the germs most frequent in small children. This panel is called “breast-fed child panel” and comprises the following germs:
Escherichia coli
Klebsiella pneumoniae
Streptococcus agalactiae
Listeria monocytogenes

These 4 germs are responsible for the great majority of bacterial infections of breast-fed children between 0 and 3 months.

### Methodologies for Performing the Test

Probes and primers as delivered are diluted to the 100 pmol/μL concentration with water for injectable preparations (sterile or apirogenic) or better.

The methodology (Realtime PCR) accurately follows the methodology of the adult panel, with the difference that probes and primers are different (Table 8)

In the “breast-fed child panel” each germ may be sought separately, by using a test tube for each couple of primers (1 test tube with primers for Escherichia coli, one test tube with primers for Klebsiella pneumoniae etc.). Alternatively, in order to use a smaller quantity of extracted DNA, it is possible to test 2 germs at a time in the same test tube by performing a multiplex Realtime PCR.

This alternative offers the advantage of needing smaller quantities of starting biological sample and therefore it is applicable also in very small children wherein the quantity of sample that may be taken may be very small.

The simultaneous research of 2 germs in the same test tube may reduce the test sensitivity. In some cases the outcome cycle of the test shifts to the right by 1-2 cycles (for example from 36 to 38). Should the quantity of germ in the starting biological sample be very low, the shift of one cycle (e.g. from 43 to 45) may make the sample appear to be negative instead of positive in the case of a very low bacterial load.

The favourite coupling is
1) Escherichia coli/Klebsiella pneumoniae
2) Streoptococcus agalactiae/Listeria monocytogenes

The alternative of using primers and probes for 2 different germs may be carried out also in the “older child/adult panel”; this alternative possesses, among its advantages:
1. reduction of the necessary DNA
2. reduction of reagent cost
3. greater convenience of test execution (smaller number of test tubes to be prepared)

Among the disadvantages, it shows a slight reduction in sensitivity, with a shift to the right of the threshold cycle by 1-2 cycles.

In order to avoid sensitivity reduction, even if minimum, use may be made of tampons which favour real-time multiplex amplification. This improvement may be used both in the “breast-fed child panel” and in the “older child/adult panel”.

TABLE 8
Primers and probes specific for “breast-fed child panel”

| | | Nome | | |
|---|---|---|---|---|
| | | Primer/ | primer/ | |
| Germ | Gene | probe | probe | Sequence |
| Kiebsiella | phosphorine E | Primer | kp for | 5′-GGCSCARTATCAGTTCGACTT-3′ |
| Pneumoniae | (phoE) | forward | | |
| | | Primer | kp rev | 5′-CCTTCGATATCCTTCCCTTTCG-3′ |
| | | reverse | | |
| | | Probe | KP | 5′FAM-TCTGCGTCCGTCCCTCGGCT- |
| | | | | 3′TAM |
| lysteria | Invasion | Primer | Lm for | 5′-AGCTGGGATTGCGGTAACAG-3′ |
| monocytogenes | associated | forward | | |
| | protein p60 gene | | | |
| | (iap) | | | |
| | | Primer | Lm rev | 5′-CAAAGAGTATCACCAGCTTCGACTAC-3′ |
| | | reverse | | |
| | | Probe | Lm | 5′JOE-TTGCTGCGCCAACAATCGCATC- |
| | | | | 3′TAM |
| Escherichia | Glucoronidase | Primer | Coil | 5′-CGGAAGCAACGCGTAAACTC-3′ |
| coli | (uidA) | forward | for | |
| | | Primer | Coli | 5′-GCGTCGCAGAACATTACATTGA-3′ |
| | | reverse | rev | |
| | | Probe | Coli | 5′JOE-ACCCGACGCGTCCGATCACCT- |
| | | | | 3′TAM |
| Streptococcus | surface | Primer | Sip for | 5′-atcctgagacaacactgaca-3′ |
| agalactiae | immunogenic | forward | | |
| | protein (sip) | | | |

| | | |
|---|---|---|
| Primer | Sip rev | 5′-ttgctggtgtttctattttca-3 |
| reverse | | |
| Probe | Sip | 5′FAM-atcagaagagtcatactgccacttc-3′TAM |

### LIST OF THE SEQUENCES USED

TABLE 9
Sequences used

| SEQ ID | | |
|---|---|---|
| No. | Sequence | Use |
| 1 | GCT GCG GTA GGT GGT TCA A | ctrA N. meningitis RT-PCR |
| 2 | TTG TCG CGG ATT TGC AAC TA | ctrA N. meningitis RT-PCR |
| 3 | GGCGAAATGGTGCTGGTAA | Bex, H. influenza RT-PCR |
| 4 | GGCCAAGAGATACTCATAGAACGTT | Bex, H. influenza RT-PCR |
| 5 | ACG CAA TCT AGC AGA TGA AGC | lytA S. pneumoniae RT-PCR |
| 6 | TGT TTG GTT GGT TAT TCG TGC | lytA S. pneumoniae RT-PCR |
| 7 | ATT ATT CAG ACC GCC GGC AG | Is1106 N. meningitis Control |
| | | PCR |
| 8 | TGC CGT CCT GCA ACT GAT GT | Is1106 N. meningitis Control |
| | | PCR |
| 9 | CTC TCA CCC TCA ACC CAA TGT C | Sia D, N. meningitis Sero B |
| | | PCR |
| 10 | TGT CGG CGG AAT AGT AAT AAT GTT | Sia D, N. meningitis Sero B |
| | | PCR |
| 11 | GCA CAT TCA GGC GGG ATT AG | Sia D, N. meningitis Sero C |
| | | PCR |
| 12 | TCT CTT GTT GGG CTG TAT GGT GTA | Sia D, N. meningitis Sero C |
| | | PCR |
| 13 | CAA ACG GTA TCT GAT GAA ATG CTG GAA G | Sia D, N. meningitis Sero W- |
| | | 135 PCR |
| 14 | TTA AAG CTG CGC GGA AGA ATA GTG AAA T | Sia D, N. meningitis Sero W |
| | | 135 PCR |
| 15 | CAGAAAGTGAGGGATTTCCATA | SynG, N. meningitis Sero W |
| | | PCR |
| 16 | CACAACCATTTTCATTATAGTTACTGT | SynO, N. meningitis Sero W |
| | | PCR |
| 17 | CTCAAAGCGAAGGCTTTGGTTA | SynF, N. meningitis SeroY |
| | | PCR |
| 18 | CTGAAGCGTTTTCATTATAATTGCTAA | SynE, N. meningitis SeroY |
| | | PCR |
| 19 | CGCAATAGGTGTATATATTCTTCC | SacB, N. meningitis Sero A |
| | | PCR |
| 20 | CTGAATAGTTTCGTATGCCTTCTT | SacB, N. meningitis Sero A |
| | | PCR |
| 21 | ACGATGCTGCAGGCAATGGT | 96, H. influenza Control |
| | | PCR |
| 22 | CATCAGTATTACCTTCTACTAAT | P6, H. influenza Control |
| | | PCR |
| 23 | TAAGAGAATTATGCAGTGCTGGC | TEM, H. influenza PCR |
| 24 | TCCATAGTTGCCTGACTCCCC | TEM, H. influenza PCR |
| 25 | CGTTTGTATGATGTTGATCCAGACT | BexA, H. influenza PCR |
| 26 | TGTCCATGTCTTCAAAATGATC | BexA, H. influenza PCR |
| 27 | AGATACCTTTGGTCCTCTGC | Cps, H. influenza PCR |
| 28 | CTTACGCTTCTATCTCGGTG | Cps, H. influenza PCR |
| 29 | GCA GTA CAG CAG TTT GTT GGA CTG ACC | CpsYABC, S. pneumoniae |
| | | Control PCR |
| 30 | GAA TAT TTT CAT TAT CAG TCC CAG TC | CpsYABC, S. pneumoniae |
| | | Control PCR |
| 31 | GTTCAACGACTAGGACGC | S. pneumoniae sero 19F PCR |
| 32 | TAGGCACCAATGTTTCACT | S. pneumoniae sero 19F PCR |
| 33 | GAG TAT AGC CAG ATT ATG GCA GTT TTA | S. pneumoniae sero 222 PCR |
| 34 | CTC CAG CAC TTG CGC TGG AAA CAA CAG | S. pneumoniae sero 222 PCR |
| | ACA AC | |
| 35 | ATC GTG TGA TTT CTC CTA GAT TGC AAA | S. pneumoniae sero 3 PCR |
| | GTA G | |
| 36 | CTT CTC CAA TTG CTT ACC AAG TGC AAT | S. pneumoniae sero 3 PCR |
| | AAC G | |
| 37 | CGACGTAACAAAGAACTAGGTGCAGAAAC | S. pneumoniae sero 6 PCR |
| 38 | AAGTATATAACCACGCTGTAAAACTCTGAC | S. pneumoniae sero 6 PCR |
| 39 | GTT ACT CCT GTT TTA GAT TTA TTT GGT | S. pneumoniae sero 19A PCR |
| | GTT G | |
| 40 | GAG CAG TCA ATA AGA TGA GAC GAT AGT | S. pneumoniae sero 19A PCR |
| | TAG | |
| 41 | GATATCCCCGGAATAAATGAAG | S. pneumoniae sero 9v PCR |
| 42 | CATGAACAAGAACGATATCAGGC | S. pneumoniae sero 9v PCR |
| 43 | CTG TTA CTT GTT CTG GAC TCT CGA TAA | S. pneumoniae sero 4 PCR |
| | TTG G | |
| 44 | GCC CAC TCC TGT TAA AAT CCT ACC CGC | S. pneumoniae sero 4 PCR |
| | ATT G | |
| 45 | GTTTATTCTATATACAAAGAGGCTCC | S. pneumoniae sero 14 PCR |
| 46 | GCATTGCAACAATCGCTATTCTAGAT | S. pneumoniae sero 14 PCR |
| 47 | GCA ACA AAC GGC GTG AAA GTA GTT G | S. pneumoniae sero 12f PCR |
| 48 | CAA GAT GAA TAT CAC TAC CAA TAA CAA | S. pneumoniae sero 12f PCR |
| | AAC | |
| 49 | CCT ACG GGA GGA TAT AAA ATT ATT TTT | S. pneumoniae sero 7f PCR |
| | GAG | |
| 50 | CAA ATA CAC CAC TAT AGG CTG TTG AGA | S. pneumoniae sero 7f PCR |
| | CTA A | |
| 51 | GGA CAT GTT CAG GTG ATT TCC CAA TAT | S. pneumoniae sero 11a PCR |
| | AGT G | |
| 52 | GAT TAT GAG TGT AAT TTA TTC CAA CTT | S. pneumoniae sero 11a PCR |
| | CTC CC | |
| 53 | GAA GGC AAT CAA TGT GAT TGT GTC GCG | S. pneumoniae sero 33f PCR |
| 54 | CTT CAA AAT GAA GAT TAT AGT ACC CTT | S. pneumoniae sero 33f PCR |
| | CTA C | |
| 55 | CTG TTC AGA TAG GCC ATT TAC AGC TTT | S. pneumoniae sero 16f PCR |
| | AAA TC | |
| 56 | CAT TCC TTT TGT ATA TAG TGC TAG TTC | S. pneumoniae sero 16f PCR |
| | ATC C | |
| 57 | GAT AAG TCT GTT GTG GAG ACT TAA AAA | S. pneumoniae sero 35b PCR |
| | GAA TG | |
| 58 | CTT TCC AGA TAA TTA CAG GTA TTC CTG | S. pneumoniae sero 35b PCR |
| | AAG CAA G | |
| 59 | CTT AAT AGC TCT CAT TAT TCT TTT TTT | S. pneumoniae sero 18f PCR |
| | AAG CC | |
| 60 | TTA TCT GTA AAC CAT ATC AGC ATC TGA | S. pneumoniae sero 18f PCR |
| | AAC | |
| 61 | CGT TCT TTT ATC TCA CTG TAT AGT ATC | S. pneumoniae sero 38 PCR |
| | TTT ATG | |
| 62 | ATG TTT GAA TTA AAG CTA ACG TAA CAA | S. pneumoniae sero 38 PCR |
| | TCC | |
| 63 | GGA AGT TTT CAA GGA TAT GAT AGT GGT | S. pneumoniae sero 31 PCR |
| | GGT GC | |
| 64 | CCG AAT AAT ATA TTC AAT ATA TTC CTA | S. pneumoniae sero 31 PCR |
| | CTC | |
| 65 | TTG GAA TTT TTT AAT TAG TGG CTT ACC | S. pneumoniae sero 15c PCR |
| | TA | |
| 66 | CAT CCG CTT ATT AAT TGA AGT AAT CTG | S. pneumoniae sero 15c PCR |
| | AAC C | |
| 67 | GAT GCC ATG AAT CAA GCA GTG GCT ATA | S. pneumoniae sero 8 PCR |
| | AAT C | |
| 68 | ATC CTC GTG TAT AAT TTC AGG TAT GCC | 5. pneumoniae sero 8 PCR |
| | ACC | |
| 69 | GGT GTA GAT TTA CCA TTA GTG TCG GCA | S. pneumoniae sero10a PCR |
| | GAC | |
| 70 | GAA TTT CTT CTT TAA GAT TCG GAT ATT | S. pneumoniae sero10a PCR |
| | TCT C | |
| 71 | GAA CAT AGT CGC TAT TGT ATT TTA TTT | S. pneumoniae sero 35f PCR |
| | AAA GCA A | |
| 72 | GAC TAG GAG CAT TAT TCC TAG AGC GAG | S. pneumoniae sero 35f PCR |
| | TAA ACC | |
| 73 | GCT TTT GTA AGA GGA GAT TAT TTT CAC | S. pneumoniae sero 34 PCR |
| | CCA AC | |
| 74 | CAA TCC GAC TAA GTC TTC AGT AAA AAA | S. pneumoniae sero 34 PCR |
| | CTT TAC | |
| 75 | CTC TAT AGA ATG GAG TAT ATA AAC TAT | S. pneumoniae sero 1 PCR |
| | GGT TA | |
| 76 | CCA AAG AAA ATA CTA ACA TTA TCA CAA | S. pneumoniae sero 1 PCR |
| | TAT TGG C | |
| 77 | TTC GTG ATG ATA ATT CCA ATC ATC AAA | S. pneumoniae sero 17f PCR |
| | CAA GAG | |
| 78 | GAT GTA ACA AAT TTG TAG CGA CTA AGG | S. pneumoniae sero 17f PCR |
| | TCT GC | |
| 79 | GAG CAA GAG TTT TTC ACC TGA CAG CGA | S. pneumoniae sero 20 PCR |
| | GAA G | |
| 80 | CTA AAT TCC TGT AAT TTA GCT AAA ACT | S. pneumoniae sero 20 PCR |
| | CTT ATC | |
| 81 | ATT AGT ACA GCT GCT GGA ATA TCT CTT C | S. pneumoniae sero 15a PCR |
| 82 | GAT CTA GTG AAC GTA CTA TTC CAA AC | S. pneumoniae sero 15a PCR |
| 83 | CTA TCT CAG TCA TCT ATT GTT AAA GTT | S. pneumoniae sero 7c POR |
| | TAC GAC GGG A | |
| 84 | GAA CAT AGA TGT TGA GAC ATC TTT TGT | S. pneumoniae sero 7c PCR |
| | AAT TTC | |
| 85 | GCATCAGTACAGTGTGCTAATTGGATTGAAG | S. pneumoniae sero 18f PCR |
| 86 | CTTTAACATCTGACTTTTTCTGTTCCCAAC | S. pneumoniae sero 18f PCR |
| 87 | ATA CCT ACA CAA CTT CTG ATT ATG CCT | S. pneumoniae sero 5 PCR |
| | TTG TG | |
| 88 | GCT CGA TAA ACA TAA TCA ATA TTT GAA | S. pneumoniae sero 5 PCR |
| | AAA GTA TG | |
| 89 | TGGTAGTGACAGCAACGA | S. pneumoniae sero 23F PCR |
| 90 | CAAAGGCTAATTCAGCATC | S. pneumoniae sero 239 PCR |
| 91 | CATTGCCACG TGTCAGCTGC ACAT | ctrA N. meningitis RT-PCR |
| 92 | CACCACTCATCAAACGAATGAGCGTGG | Bex, H. influenza RT-PCR |
| 93 | TTTGCCGAAAACGCTTGATACAGGG | lytA S . pneumoniae RT-PCR |
| 94 | TGCAGAGCGTCCTTTGGTCTAT | ply S. pneumoniae RT-PCR |
| 95 | CTCTTACTCGTGGTTTCCAACTTGA | ply S. pneumoniae RT-PCR |
| 96 | TGGCGCCCATAAGCAACACTCGAA | ply S. pneumoniae RT-PCR |
| 97 | CTGAATTGGGCGATTATCTTTATGA | Capsula, BexA, H. influenza |
| | | PCR |
| 98 | ACAATCAAACTCAACCGAAAGTGA | Capsula, BexA, H. influenza |
| | | PCR |
| 99 | GGACGCCTCGGAGTACCTGAG | Kobe H, Adenovirus RT-PCR |
| 100 | ACCGTGGGGTTTCTGAACTTGTT | Kobe H, Adenovirus RT-PCR |
| 101 | CTGGTGCAGTTCGCCCGTGCCA | Kobe H, Adenovirus RT-PCR |
| 102 | GGCGCAGTATCAGTTCGACTT | phoE, Klebsiella P RT-PCR |
| 103 | CCTTCGATATCCTTCCCTTTCG | phoE, Klebsiella P RT-PCR |
| 104 | TCTGCGTCCGTCCCTCGGCT | phoE, Klebsiella P RT-PCR |
| 105 | AGCTGGGATTGCGGTAACAG | p60, Lysteria RT-PCR |
| 106 | CAAAGAGTATCACCAGCTTCGACTAC | p60, Lysteria RT-PCR |
| 107 | TTGCTGCGCCAACAATCGCATC | p60, Lysteria RT-PCR |
| 108 | CGGAAGCAACGCGTAAACTC | uidA, E. coli RT-PCR |
| 109 | GCGTCGCACAACATTACATTGA | uidA, E. coli RT-PCR |
| 110 | ACCCGACGCGTCCGATCACCT | uidA, E. coli RT-PCR |
| 111 | ATCCTGAGACAACACTGACA | sip, S. agalactiae RT-PCR |
| 112 | TTGCTGGTGTTTCTATTTTCA | sip, S. agalactiae RT-PCR |
| 113 | ATCAGAAGAGTCATACTGCCACTTC | sip, S. agalactiae RT-PCR |
| 114 | AATTTGTATTTTATTCATGCCTATATCTGG | S. pneumoniae sero 6A-6B PCR |
| 115 | TTAGCGGAGATAATTTAAAATGATGACTA | S. pneumoniae sero 6A-6B PCR |
| 116 | GGTGCATTCGCAGCTTCAG | p2 H. influenza RT-PCR |
| 117 | GATTGCGTAATGCACCGTGTT | p2 H. influenza RT-PCR |
| 118 | TTGTTTATAACAACGAACGCGCTAACGT | p2 H. influenza RT-PCR |
| 119 | AGCGTGTGTTGGATGCGCTGGCTGCGGTAGGTGGT | ctrA N. meningitis RT-PCR |
| | TCAACGGCAAATGTGCAGGATACGAATGTGCAGCT | target |
| | GACACGTGGCAATGTAGTACGAACTGTTGCCTTGG | |
| | AAGATTTAGTTGCAAATCCGCGACAAAATATTTTG | |
| | CTGCGTCGCGG | |
| 120 | CACTTGCAGC ATTAATCGTT GGTGCATTCG | P2, H. influenza RT-PCR |
| | CAGCTTCAGC AGCTAACGCAGCTGTTGTTT | target |
| | ATAACAACGA AGGGACTAAA GTAGAATTAG | |
| | GCCGTCGTGT AAGTATTATTGCAGAACAAA | |
| | GCACTAGCAC TGGAGATGAT CAAAAACATC | |
| | AACACGGTGC ATTACGTAATCAGGGTTCAC | |
| | GTTTCCATAT T | |
| 121 | CCAAA AAATCGGGAT TTTAGGGCGA | Bex, H. influenza RT-PCR |
| | AATGGTGCTGGTAAATCCAC GCTCATTCGT | target |
| | TTGATGAGTG GTGTTGAGCC TCCAACAAGT | |
| | GGTACGATTGAACGTTCTAT GAGTATCTCT | |
| | TGGCCGTTAG CTTTTAGTGG GGCAT | |
| 122 | CGCCTTTAT ATCGAACTCT TACGCAATCT | lytA S. pneumoniae RT-PCR |
| | AGCAGATGAA GCAGGTTTGC | target |
| | CGAAAACGCTTGATACAGGG AGTTTAGCTG | |
| | GAATTAAAAC GCACGAGTAT TGCACGAATA | |
| | ACCAACCAAA | |
| | CAACCACTCA GACCATGTGG AT | |
| 123 | TTAAAACAGA GAGGAATTTCTGCAGAGCGT | ply S. pneumoniae RT-PCR |
| | CCTTTGGTCT ATATTTCGAG TGTTGCTTAT | target |
| | GGGCGCCAAG TCTATCTCAAGTT GGAAACC | |
| | ACGAGTAAGA GTGATGAAGT AGAGGCTGCT T | |
| 124 | TACATGCACATCGCCGGGCAGGACGCCTCGGAGTA | Adeno RT-FCR target |
| | CCTGAGCCCGGGTCTGGTGCAGTTTGCCCGCGCCA | |
| | CCGACACGTACTTCAGCCTGGGCAACAAGTTTAGA | |
| | AACCCCACGGTGGCTCCCACCCACGATGTGA | |
| 125 | CTTTAATTTATACACCATTACTCTCACCCTCAACC | Sia D, N. meningitis Sero B |
| | CAATGTCTTTCTATTGGAGAGTTAATTATTAACTT | PCR target |
| | AATTCAAAAATATTCAATGGTGGAAAACACTGAAA | |
| | TGATCCAAGAACACTTAGAGATTATTAAGAA | |
| | ATTTAATTTTATTAATATACTAAATGATTTAAATG | |
| | GGGTAATAAGTAACCCTCTCTTTAAAACAGAAGAA | |
| | ACATTTGAAACACTTCTTAAATCTGCAGAATTCGC | |
| | ATATAAATCTAAAAACTACTTTCAGGCTATTTTTT | |
| | ACTGGCAACTTGCCAGCAAAAACAATATTACCTTA | |
| | TTAGGGCATAAAGCATTATGGTACTAGAATGCACT | |
| | TTATAATGTAAAAGCAAATTTATAAGATGGA | |
| | ATATTCAGATATTTTTTATATCGATAATATCTCCG | |
| | TAGACTTTCATAGTAAAGATAAATTGACATGGGAA | |
| | AAAATTAAACATTATTACTATTCCGCCGACAATAG | |
| | AATTGGTAGAGATAGA | |
| 126 | ATGAATACCACCGTTTTTTTGCACATTCAGGCGGG | Sia D, N. meningitis Sero C |
| | ATTAGCACAAGCCAATCTATTGCTAAAATTCAAGA | PCR target |
| | CAAATACCGCATATCTCAAAATGACTATATTTTTG | |
| | TAAGTCAACGCTATCCTCTTTCAGATGAAGTTTAT | |
| | TATAAAACTATTGTCGAAACATTAAATCAAATGAG | |
| | TTTGCGAATAGAAGGTAAAATTTTTATTAAACTCC | |
| | ATCCTAAAGAAATGGAGAATAAAAATATTATGTCT | |
| | TTATTCCTGAATATGGTAACAATTAATCCCCGTCT | |
| | AGTAGTGATTAATGAACCCCCTTTTCTCATTGACC | |
| | CACTAATTTATTTAACAACCCCTAAAGGAATTATT | |
| | GGACTGACATCGACTTCTATTGTTTATACACCTTT | |
| | ACTTTCCCCTACTACCCAATGTCTGTCAATTGGGC | |
| | AAATCGTGATTGATTCGATACACCATACAGCCCAA | |
| | CAAGAGAACGCCGCTTTAATTGAAGAG | |
| 127 | TCGATTATGTTGACTATCTTCAAACGGTATCTGAT | Sia D, N. meningitis Sero W- |
| | GAAATGCTGGAAGAAATGCATTCCCATTTCAAAAT | 135 PCR target |
| | CAAAAAAGACAAATTAGTTTTTATTCCAAACATCA | |
| | CTTATCCCATTTCATTAGAAAAAAAAGAAGCTGAT | |
| | TTCTTTATTAAGGATAATGAAGACATCGATAATGC | |
| | TCAGAAATTTAAACGTATCTCTATTGTTGGCAGCA | |
| | TTCAGCCAAGAAAAAACCAATTGGATGCCATTAAA | |
| | ATCATCAATAAAATTAAAAATGAAAATTACATTTT | |
| | ACAGATATATGGCAAATCTATTAATAAAGATTACT | |
| | TTGAATTAATTAAAAAATATATTAAAGACAATAAG | |
| | TTACAAAACCGTATCTTATTCAAAGCTGAATCTTC | |
| | CGAGCAGGAAATTTATGAAAATACAGATATCCTGA | |
| | TCATGACATCAGAAAGTGAGGCATTTCCATATATA | |
| | TTTATGGAAGGCATGGTGTATGATATTCCAATCGT | |
| | TGTATATGATTTTAAATATGGAGCGAATGATTACA | |
| | GTAACTATAATGAAAATGGTTGTGTTTTTAAAACT | |
| | GGTGATATTTCTGGAATGGCAAAAAAAATAATTGA | |
| | GCTATTAAATAACCCAGAAAAATATAAAGAATTAG | |
| | TTCAATATAATCACAATCCCTTCTTAAAAGAATAT | |
| | GCAAAAGATGTGGTTATCGCTAAATATTTCACTAT | |
| | TCTTCCGCGCAGCTTTAATAACGTATCATTATCGT | |
| | CTG | |
| 128 | AGATATCCTGATCATGACATCAGAAAGTGAGGGAT | SynC, N. meningitis Sero W |
| | TTCCATATATATTTATGGAAGCCATGGTGTATGAT | PCR target |
| | ATTCCAATCGTTCTATATGATTTTAAATATGGACC | |
| | GAATGATTACAGTAACTATAATGAAAATGGTTGTG | |
| | TTTTTAAAACTGGTGATATT | |
| 129 | AGATATCCTAATCATGACATCTCAAAGCGAAGGCT | SynF, N. meningitis SeroY |
| | TTGGTTATATATTTCTAGAGGGTATGGTGTACGAT | PCR target |
| | ATCCCTATCCTTGCCTATAATTTTAAATATGGAGC | |
| | GAATGATTTTAGCAATTATAATGAAAACGCTTCAG | |
| | TTTTTAAAACTGGTGATATT | |
| 130 | AAAAAACTTAACAATCAAAACGCAATAGGTGTATA | SacB, N. meningitis Sero A |
| | TATTCTTCCTTC | PCR target |
| | TAATCTTACTCTTAAGCCTGCATTATGTATTCTAG | |
| | AATCACATAAAGAAGAGTTTTTAAATAAATTTCTT | |
| | CTTACTATTTCCTCTGAAAATTTAAAGCTTCAATA | |
| | CAAATTTAATGGACAAATAAAAAATCCTAAGTCCG | |
| | TAAATGAAATTTGGACAGATTTATTTAGCATTGCT | |
| | CATGTTGACATGAAACTCAGCACAGATAGAACTTT | |
| | AAGTTCATCTATATCTCAATTTTGGTTCAG | |
| | ATTAGAGTTCTGTAAAGAAGATAAGGATTTTATCT | |
| | TATTTCCTACAGCTAACAGATATTCTAGAAAACTT | |
| | TGGAAGCACTCTATTAAAAATAATCAATTATTTAA | |
| | AGAAGGCATACGAAACTATTCAGAAATATCTTCAT | |
| | TACCCTAT | |
| 131 | TCG TTTTACAAAA GAGTTTTCTGAATTGGGCGA | Capsula, BexA, H. influenza +l |
| | TTATCTTTAT GAGCCAGTGA AGAAGTACTC | PCR target |
| | ATCAGGGATG AAAGCCCGACTTGCATTTGC | |
| | TCTCTCACTT TCGGTTGAGT TTGATTGTTA | |
| | TTTAATTGAT GAGGTGAT | |
| 132 | TCTTACGGATGGCATGACAGTAAGAGAATTATGCA | TEM, H. influenza PCR |
| | GTGCTGCCATAACCATGAGTGATAACACTGCTGCC | target |
| | AACTTACTTCTGACAACGATCGGAGGACCGAAGGA | |
| | GCTTACCGCTTTTTTGCACAACATGGGGGA | |
| | TCATGTAACTCGCCTTGATCGTTGGGAACCGGAGC | |
| | TGAATGAAGCCATACCAAACGACGAGCGTCACACC | |
| | ACGATGCCTGCAGCAATGGCAACAACGTTGCGCAA | |
| | ACTATTAACTGGCGAACTACTTACTCTAGCTTCCC | |
| | GGCAACAATTAATAGACTGGATGGAGGCGGATAAA | |
| | GTTGCAGGACCACTTCTGCGCTCGGCCCTTCCGGC | |
| | TGGCTGGTTTATTGCTGATAAATCTGGAGCCGGTG | |
| | AGCGTGGGTCTCGCGGTATCATTGCAGCACTGGGG | |
| | CCAGATGGTAAGCCCTCCCGTATCGTAGTTATCTA | |
| | CACGACGGGGAGTCAGGCAACTATGGATGAACGAA | |
| | ATAGACAGATCG | |
| 133 | ATAATCTGCGTTTTATTTGTCGTTTGTATGATGTT | BexA, H. influenza PCR |
| | GATCCAGACTACGTTACTCGTTTTACAAAAGAGTT | target |
| | TTCTGAATTGGGCGATTATCTTTATGAGCCAGTGA | |
| | AGAAGTACTCATCAGGGATGAAAGCCCGACTTGCA | |
| | TTTGCTCTCTCACTTTCGGTTGAGTTTGATTGTTA | |
| | TTTAATTGATGAGGTGATTGCAGTAGGGGATTCGC | |
| | GTTTTGCAGAAAAATGTAAGTATGAGTTATTTGAG | |
| | AAACGCAAAGACCGTTCTATCATTTTAGTTTCACA | |
| | TAGCCCGAGTGCGATGAAATCTTATTGTGATAATG | |
| | CGGTAGTGTTAGAAAATGGTATTATGCATCATTTT | |
| | GAAGACATGGACAAAGCGTATCAATAC | |
| 134 | TGGGGGCGAGGCTATACACAAGATACCTTTGGTCG | Cpa, H. influenza PCR |
| | TCTGCTAAATGATGCCTTTGGTAAAGAAGTAAAAA | target |
| | ACCCATTCTATTATGTCAGAAGTTTTACTGATGAT | |
| | ATGGGTACATCTGTTCGCCATAACTTCATCTTAGC | |
| | ACCACAAAACTTCTCATTCTTCGAGCCTATTTTTG | |
| | CACAAACCCCATACGACACTATTCCTGATTACTAC | |
| | GAAGAAAAAGGCAGAATTGAACCAATTA | |
| | TTAATCACCGAGATAGAACCGTAAGCGATCTCATT | |
| | TCGGAAGGGT | |
| 135 | CTCTTTGTTTTTA GCCGGTCGTT CAACGACTAG | S. pneumoniae sero 19F |
| | GACGCTATTT GAGAGTGTTT | PCR target |
| | CGACCTATCTAGGAGGCTCA ATTCAGCATT | |
| | TTAATCAGTA TATTGAAAAT CCATTAGATC | |
| | CTGGTGAAGT | |
| | TTTTGGCAGT GAAACATTGG TGCCTATATT | |
| | AAATATATTA GGGGAA | |
| 136 | ATGATAAAATAATTAT TTCCGAGTAT | S. pneumoniae sero 22F |
| | AGCCAGATTA TGCCAGTTTT ATTGTCTAGA | PCR target |
| | ATTCATTCCA ATGTATATAT TTATAATGGT | |
| | CCATACTATA ATCTATTTAA GATCCCCATT | |
| | ATTGAATCTA TGTACGATTT TCTTTTTGTA | |
| | AGAATGCTCA ATAAGAGAAC CAAGACAGTT | |
| | TTCTGCAAGA CAGAAAAGGC AAAGCATTAT | |
| | CTTAAAAATA AGGGATTCAA TGATTGTAAA | |
| | GTAGTGGGCG TAGGTCTAGA TGTTGAAAAG | |
| | TTTGAACAAG AAGAGGAACC TACGGAGAAT | |
| | ACTATAGAAC TTTTAAAAAG AATGGAGAAT | |
| | AAGCAAAATA TTCTTTATGT AGGCTCACTA | |
| | TCAAAAAGAA AAAACACAGC TCATTTAATT | |
| | AGAATTTTTA ATATTTTAAA ATCTAAAAGT | |
| | GGTAAGAAAA ACGAACTTCA ATTGGTATTG | |
| | ATTGGTAAGG ATGAGGGTAA TATTGTTGAA | |
| | AAAATTAACT ATTCACCGTT TAAAGATGAT | |
| | ATTATTTATC AACCTTATTT AAAGAACTCT | |
| | CAGCTTCAAT TTATTTACCC ATCATCACAA | |
| | CTATTTGTGC TCCCCTCGAT TCAAGAGATT | |
| | TTTGGTATGG TATTACTTGA GGCAATGTAT | |
| | TTTAAGTTGT CTGTTGTTTC CAGCGCAAGT | |
| | GCTGGAGGAG AGACACTGAT TCAAGAT | |
| 137 | ATATGAAGATGTTTCTTCTTATGGTGTGATTTCTC | S. pneumoniae sero 3 PCR |
| | CTAGATTGGAAAGTAGTAATGGCCTCTATAGTGTT | target |
| | GATGCTTTTGTAGAGAAACCAAAACCAGAAGAAGC | |
| | GCCTAGCCATTTAGCTATTATTGGACGTTATCTAC | |
| | TTACTCCTGAGATTTTTTCTATATTAGAAACCCAA | |
| | AAGCCAGGAGCAGGTAATGAAATTCAATTGACAGA | |
| | TGCTATTGATACATTGAATAAGACACAGAGTGTTT | |
| | TTGCGCGTGAATTTGTGGGCAAACGTTACGATGTT | |
| | GGTGATAAGTTTAATTTTATGAAAACATCAATTGA | |
| | TTATGCTCTTCAACATCCTCAGATTAAAGAGAGTT | |
| | TAAAAAATTACGTTATTGCACTTGGTAAGCAATTG | |
| | GAGAAGCTAGATGACTGTTCGTCAAG | |
| 138 | TATTTGTT ATAGATCCGA TACGACGTAA | S. pneumoniae sero 6 PCR |
| | CAAAGAACTA GGTGCAGAAA CTTTTTCGGG | target |
| | AATTTATGAG ATGCTTGCAA AATTAGGATT | |
| | TGACAATAAT ATTATAAAAG GCTTAGAATG | |
| | GAGAGTGTCT CCTAATTATT ATTCTTTAGG | |
| | GAATGTGTAT ACTGCAATTA GACCTTATTA | |
| | TTCAGACTTT GGTGTAATTG GTATTGTAAT | |
| | TTGTCAGAGT TTTACAGCGT GGTTATATAC | |
| | TTTAGGTTAT GAAAAAGTTA GA | |
| 139 | CTGGACAAC ACAAAGAGAT GGTTAGTCCT | S. pneumoniae sero 19A |
| | GTTTTAGATT TATTTGGTGT TGTACCAGAT | PCR target |
| | TATGATTTAG AAATTATGAA GGCTAACCAA | |
| | ACCTTGTTCT CTATCACAAC TAGTATCTTG | |
| | GAAAAGATAA AACCAGTTTT AGAGAAGGAA | |
| | CAACCAGATA TTGTCCTAAT TCACGGTGAC | |
| | ACTACGACAA CTTATGCAGC AGCCTTGGCA | |
| | GCATTCTATT TGGGAATTAA AGTAGGACAT | |
| | GTTGAAGCTG GTTTGCGAAC GTACAATTTA | |
| | CAAAGTCCAT TTCCTGAAGA ATTTAACAGG | |
| | CAATCGACAT CAATCATTGC AACTTACCAT | |
| | TTTGCTCCAA CTGAGTTGGC TAAAGAAAAT | |
| | CTCTTAAAAG AAGGTAGAGA GAATGTTTAT | |
| | GTGACTGGAA ATACTGTCAT TGATGCTCTT | |
| | ACAACTACTG TTCAAGAGGA TTATACACAC | |
| | ACTCATTTAG ATTTAAACGC TAACAATCGT | |
| | CTCATCTTAT TGACTGCTCA TAGACGCGAA | |
| | AATCTCGGC | |
| 140 | GGAAAA CGATGGTTGC ATCAGATATC | S. pneumoniae sero 9v |
| | CCCGGAATAA ATGAAGTAGT CAATAACAAA | PCR target |
| | AATGGGATTC TTGTTCCTGT TAAGGATGAT | |
| | GTTGCCTTAG CGAGAGCGAT TGAAAAGCTT | |
| | GCGACTGACA AAAAACTTAG AGAAAAACTC | |
| | GCTTATCAAG CAAAAAAAGA CTATGAGACA | |
| | AAATTTAATT ATTCCTTGTT TTTGGATAAT | |
| | TATCGGAGAC TTTATAGAAA ATTAACGGGA | |
| | GAATCAAAAT GAAAAAAGTC ATGTTGGTTT | |
| | TTGGGACACG TCCAGAGGCT ATTAAAATGT | |
| | GTCCCCTAGT AAATGAACTG AAACAAAATG | |
| | ATTCAATCAA GACACTTGTG TGTGTAACTG | |
| | GTCAACATAA GGAAATGCTA GAGCAGGTTT | |
| | TGGAGGTGTT CAAGGTTGTC CCAGATCATG | |
| | ATTTGGGTAT CATGAAAGCA AATCAAACCC | |
| | TATTTTCCAT TACGATTAGT ATTTTGGATA | |
| | AGATTCAAGC AGTTCTAGAA CAAGAAATGC | |
| | CTGATATCGT TCTTGTTCAT GGAGATACAA | |
| | CAACAACATT T | |
| 141 | TTAAG TTTTATATTT GCTTTCTGTT | S. pneumoniae sero 4 PCR |
| | ACTTGTTCTG GACTCTCGAT AATTGGAATA | target |
| | ATGGTTTTCT TATATCTAAT TATGTTCCGT | |
| | CTATATTTAT ATGGGTTTGC TTTCTAATTA | |
| | TTTTTCAAAT TACTGGTTTT ATTTTACAAA | |
| | AAGTTAGTAT ATATGATTTT TCTGTATGGT | |
| | ATCTGATTTT ATCTTATTTT TTTATGTTTG | |
| | GATTAATTTT CAATGAGTAT ATGGGGTTTC | |
| | AAACAACTCT GCTGTGGAGC CCTAGTAACT | |
| | TCTATAATAA TGAAGAATTA TTTCATTCAT | |
| | ATATTTTTAT AATTTGGATT TTGTTTTGTT | |
| | ATTCTGTAGG CTATTTATTT TTTTATAGTG | |
| | ATGGAAAGGT ACATTATCAT TCAGAAGTAC | |
| | AAAATTATCA GGAAAATGAA GAGAAAATTT | |
| | TGTACAATGC GGGTAGGATT TTAACAGGAG | |
| | TGGGCTTTAT TTCTAGGGTA ATAAC | |
| 142 | TATCT TAGTTGTTTT TGTCGGTTTA | S. pneumoniae sero 14 |
| | TTCTATATAC | PCR target |
| | AAAGAGGCTC CAATGTAATT TTGTTTGGAA | |
| | GAAGTTTGTT AGACTGGGAC GGATTTACAT | |
| | TAGCTACTAG TTATGGTGTA AGATATACAG | |
| | GTTTTTTAGA ATACGCAACT TTAAATGGTC | |
| | AGTTAATTCT TTTTTTATTA CCGTTAATTA | |
| | GATTGTTTAG ATTTAGATTT TTTACACAAA | |
| | CTATCATTTT TGCTTTTCTT CTAGAGGTTT | |
| | TGGTACTAAG CAAATCTAGA ATAGCGATTG | |
| | TTGCAATGCT TATATATATA GCATTTGCA | |
| 143 | TGCC AACAATTTTA ATGTTGGCAA | S. pneumoniae sero 12f |
| | CAAACGGCGT GAAAGTAGTT GGAACTGACT | PCR target |
| | ACAATCAGGA TTTGGTGAGA ACTTTAAATG | |
| | AAGGTCAAAC AACCTTTAAA GAAGATGGAT | |
| | TGGATGAACT ATTCCATAAAGCAGTGGAGT | |
| | CGGGTGTGGA CTTTACAACC GAATATCAAC | |
| | AAACGGATAC CTATATTATT TCCGTTCCAA | |
| | CACCATATGA CTCCTTCTCT AAAAAAATTG | |
| | ATCCAAGCTA TGTGATTGAAGCTACGAAAA | |
| | CGGTACTTGA TAATTGCAAT AAAGGAGCGG | |
| | TTATTATTAT TGAATCGACC GTATCACCAG | |
| | GAACGGTTGA TAAATTTATT CGACCTGTTG | |
| | TAGAAGAAAA AGGTTTTGTTATTGGTAGTG | |
| | ATATTCATCT TGTCCATGCT CCAGAACGTA TT | |
| 144 | TACCT GAGATAAGCA CTGTTCCTAC | S. pneumoniae sero 7f PCR |
| | GGGAGGATAT AAAATTATTT TTGAGTACGC | target |
| | AAATAGGTTG TCTGAACGAG GTCATGAGAT | |
| | AACCCTTGTT TTTTTAACCA ATAATGTATG | |
| | GAACAGAGTT ACTAAGAATT GTAAAATCAA | |
| | ATCAATTGTA GGCAATATAC GTGGCAAAAA | |
| | AAATCCAAGT TGGTTTAAAC TGAAACCAAC | |
| | TATTCGTAAA ATAATGACTC CTTATTTAGA | |
| | TGGGAGAGAT TTTCCTGAAG CAGATTTTAT | |
| | ATTTGCTACT GCGGTTACAA CTGCTAATAT | |
| | TGTAAAAGAA ATGCCAGAAA AATATGGGAA | |
| | AAAATGTTAC CTAATCCAAG GTTTTGAAAC | |
| | ATGGCTTCTT CCAGAAAGTA AAGTTATTGA | |
| | AACTTATAAT TATGGATTCC TTAATATTAC | |
| | AGTATCTAAG TGGTTATGTG ACATAGTCCA | |
| | ATCTTATACA GAGACTCCAG TTTTTTGTGT | |
| | ATCTAATCCG ATTGATACAG AAATTTTTTA | |
| | TTTGCTAAAT CCTATAGAGA AACGAAACCC | |
| | ATTTCATCTA GGTATGTTAT ATCATGAAGG | |
| | AGAGCATAAA GGAATCTCTT ATGCGATAGA | |
| | TGCAATTAAG AAAGTTAAGA AAATTTATCC | |
| | GGAAATAGAG GTTAAAATTT TTGGAGTTCC | |
| | TAGTAGACCA GTATTTTTAC CAGAATATTT | |
| | TAATTATACT CAACAAGCTA CACAACAGGA | |
| | ATTACAAAAG ATATACAACG ATACGTCTAT | |
| | TTTTTTGTGT GCAACAATTG ATGAAGGCTT | |
| | TGGTTTGACA GGAGCAGAGA GTATGGCTTG | |
| | TGGCTGTGCG TTAGTCTCAA CAGCCTATAG | |
| | TGGTGTATTT GAATATGCAA TTGATGGCGA G | |
| 145 | T TTTTCATATT ACTTTTTTAG GACATGTTCA | S. pneumoniae sero 11a |
| | GGTGATTTCC CTATATAGTG TTATCGGATT | PCR target |
| | TCTCGTATCA GCATATTACT | |
| | TATTAGAAGA AAAGAGAAAT ATATTAGTGG | |
| | CACAGTTGCT GTTGATTTTA ACGATTATCA | |
| | ATTGTTTTTT TTCAGATGTC TCTCTCTCTA | |
| | AAGTTATTGC TTTGTTTATG ATTGTTTATA | |
| | TTATTTCATA CAAGTTAAGA AAACTATTCT | |
| | GGAAAAGAGG AAGGAAGATA TCTGTTGTAA | |
| | CTTTTATTGC ATCTGTTGTG ATGTTAGCAT | |
| | TAGTAATTTT TGGATATTTT TTACCATATC | |
| | TTAGATATTT TGATTTTACT TTTAATGGTC | |
| | GATACCAAAT TTGGCGCATT GTGTATGCTA | |
| | CCATTCTTCA AGTGAAATGG TTTGGATATG | |
| | GTTTGTTTGG CTTTCAATTT AAACTTCCGT | |
| | GGCAGAAATT GGGAGAAGTT GGAATAAATT | |
| | ACACTCATAA TCAAGTTTTA CAGTTAGCAC TT | |
| 146 | CT TATCTCTAGA ATATCTATGA AGGCAATCAA | S. pneumoniae sero 33f |
| | TGTGATTGTG TCGCGTTCTT TAATATTTAT | PCR target |
| | ATTAATTATT CTACTCATAG TAATATTAAA | |
| | TGGTTTTAAG ATTTCTGAGA CAAGTTTCGT | |
| | CTATTATTTT GTATTATTTC CGATTTTTAT | |
| | GATGATTTTG CAGATGTACT ATGATGTTAA | |
| | TGAAATCGCA AATCTGATAC GGAAATTTGT | |
| | TCGTATAATA TTTCTTTTAG CAATTGGCTC | |
| | TCTCCTATTT TGGCTTATTG GTAGTGTATT | |
| | TCATATTATA TCCCCAACGG TTTATGTGTT | |
| | GAATTATTGG AATGGTGGGG GAATAGTAGA | |
| | AGGGTACTAT AATCTTCATT TTGAAGCACA | |
| | AAAAATAGAG ATTTTG | |
| 147 | ATTGGTTTAG TTTATACTAT CTGTTCAGAT | S. pneumoniae sero 16f |
| | AGGCCATTTA CAGCTTTAAA TCTTTTTAGA | PCR target |
| | ATTTATATGT TTCCTGCTGT | |
| | AATATATTTT ATAATAATAA ATTGTAATTT | |
| | TCGCAAGGAG AGATTACTGA TATTGAAGCA | |
| | AGCGCATGTA TATACTGCTT GCATATTAGC | |
| | TTTATGGGGA ATTTTTCAGG CGTGGGTGTT | |
| | AAAAGATCAG TTTTTGATTA AAATAGGATA | |
| | TCCTTCCCAA GGAAATTTTC TAAAGAGTAC | |
| | AGCTTTTTAT ATTGGTGGTT TTTTCGGTCA | |
| | ACAACGAGTT ACTAGCACTT TTTCTGCTCC | |
| | TAATTTAGCT GGAGTTTATT TTGGAATATC | |
| | TTTGATCATT TTACTCTCTA TATTTGATAC | |
| | TATAAAGAGT AATAGATTGG TATTGTTTTC | |
| | AATTGTAGCA GCTTTTGTTC TTACATTTAG | |
| | CCGTAGTGCT ATTATAAGTA CTCTTGTAGG | |
| | AATCGTATTC TTTCAACGTA AGAAATTGTT | |
| | TTCAACGACG AAGATTAATG TTATGACCTT | |
| | GGTAATCTTC CCCTTAATTT TTTTGATTGT | |
| | TTTAGTTATA TTTTATCTTT ATCCTGAGAA | |
| | TGTTATTATA AATATGCTCT ATAGCAGTTA | |
| | TTCTTCAACA CTTAATTTGA CAGATTCTTC | |
| | AGCGGTTAAG CATTTGGAAG ACTTATGGCT | |
| | TCCTCTTCTA AAAGTTATTG ATTATCCTTT | |
| | GGGACTTGGA TTTGGTAATA ATGGCCCAAT | |
| | TGTTCTGTCA CTCTATCACT CAGCAAATTT | |
| | AGTAGAATCT TCTATTTATC TTCTTGCATA | |
| | TGATTTTGCT ATTTTAGCAA TGTTCATATA | |
| | TTTGTTCCCT TATTTTTATA CGATTTTTGT | |
| | ATATAAAAAA TATTTGCTTA GCGGTGCTAT | |
| | ATGCTGCTTA GTTCTTATAA CTTATCTATT | |
| | TCTTCCAAAT GTAGAAAATT TTGAAATCAT | |
| | ATTTTTTATT TACCTTTTCA TTGGGATGGA | |
| | TGAACTAGCA CTATATACAA AAGGAATGAA | |
| | TGTACATGAA ATTGATTA | |
| 148 | TGTGT TGTAGGTTAT AATTGGATAA | S. pneumoniae sero 35b |
| | GTCTGTTGTG GAGACTTAAA AAGAATGATG | PCR target |
| | TAGTTATTTA CCAACATCCA ATGTACGGAG | |
| | TGCGTGTTGC AAATTTTGCA ATTCCTTTAT | |
| | TGAAAAAGTA TAAAAATATA AAATTTATTT | |
| | CAGTAATACA TGATTTAGAA TCGTTAAGAA | |
| | AAGGAATCCA AGGAGTCATT GAAGATAATG | |
| | AAACAACAAA TGCTATTGCA GACAAAGAGT | |
| | TGTTGTCAAA ATTTGATAAA GTGATTTCCC | |
| | ATAATCCGAA AATGACAGAA TATTTAGAAG | |
| | GGATAGGGAT AAAAAAAGAA AATTTAGTTG | |
| | AATTGCAAAT ATTCGACTAC CTAGATCCAT | |
| | CAGAAATAGA AGAAAAGATT GAAGATGGAG | |
| | TGGTTATAGC GGGGAATCTA GCAAAAGGAA | |
| | AAAGTTCATA TATATATAAG TTGTTAGAAA | |
| | ATGAACTGAA CTTCAAATTA AATCTTTTTG | |
| | GGCCAAATTT TATTAACGAA GAGCTACCAG | |
| | AAAACGTTGA GTATTTTGGT AGTTTACCTC | |
| | CAAATAAATT GCCTCAAAAG CTAGTAGGTA | |
| | AGTTTGGTTT GGTTTGGGAC GGTGATAGTC | |
| | TAGAAACTTG TAGTGGAAAT ACTGGTAACT | |
| | ATTTGAAGTA TAATAATCCA CATAAAACCT | |
| | CATTATATCT TGCTTCAGGA ATACCTGTAA | |
| | TTATCTGGAA AGAAGCTGCG TTAGCACAGT TT | |
| 149 | TCT TCAGTATTTC ATAATATCTT | S. pneumoniae sero 18f |
| | AATAGCTCTC ATTATTCTTT TTTTAAGCCT | PCR target |
| | TAAACGAATC GTTTTTTTGA GTGTATTAAT | |
| | TATCATACCA GTATTTTTGG TAATTTATTG | |
| | GTATGATAAA AAAGTAAGCA AACTAGGGAA | |
| | AGAACGAAAA ATTTTAAGTT TATTAAATAT | |
| | CTTTTCCTTA ATATTTATAA CAGGAATATT | |
| | CTTTTATGTT TATAGTGTAA AATCTGATTT | |
| | TATATATACA TTTATTCAAG AACATAATAT | |
| | TAATTCGATG GCTAGAACAG ATTTATGGAA | |
| | GGGAGTTGAA TCAACCTATA ATTTCGCCCC | |
| | TATATTTATG GGGAGAGGGA TAGGGTTTGT | |
| | AACAAAATGG ATGGATAATA ATTGGATGAC | |
| | TTTGAATATC AATGGTCTTA CAGGATCAAT | |
| | GGGGATCCAT AATGATATTT TGAAGTACTA | |
| | CATTCAGATA GGATTTGTAG GATTATTTAT | |
| | TTATTTTTAC ACTCTTCTTT ATAGAAATGC | |
| | TAAACGTATA TTTGTAAAAA TTGGTCATAA | |
| | AGAATCATTC ATATATTTTG TATTGATAAT | |
| | GTTTCAGATG CTGATATGGT TTACAGATAA | |
| | TATTTCAATT TACCATAATT | |
| 150 | TAT ATGTATCTTA TTTAAATCGT TCTTTTATCT | S. pneumoniae sero 38 |
| | CACTGTATAG TATCTTTATG ATTGTATTTT | PCR target |
| | ACCTTTTTCA AAATGGACAG GTATTGTTAT | |
| | ATTCACTTGG AGTAGAGTAT GATTACTTTT | |
| | ATGTTTTAAG ATATGATGAA ACCATCGTAC | |
| | TACAATCAGT TATTTTTTCA ACACAATGTC | |
| | TTATTGCAGC CTTTATGGCT GGAGTCTTTT | |
| | CTACGAAGAA AGAGGTATCA AAACCCCTTT | |
| | ATTCTTATAT GGATCAATTA GAACGAGAAA | |
| | AACTGATTAC TACTGGAAAG TTATTTTGGG | |
| | GTGCGTTTGC GATTTTTGCT TTACCCTTTA | |
| | TGATGATGAA GCTTGTTATC ACCAGTACAT | |
| | CTGGGTATTT TGCGATGATA CGTTTCCTTG | |
| | GAAGCCTACC TACAATAACA GTGTTATTTG | |
| | AAAAAATGTT CATTGCTTCA AGTGTTTTTT | |
| | TGATTGTTTA TTTAAAATCT GAGGAAACTT | |
| | GGAGCAAGTT TTTAAAAGTT GTTATCTTAG | |
| | GTTGGAGTAT AATGGCTGCT TTAACAGGAG | |
| | ATAGAACCGT AGGTTTAGCA GGGATTGTTA | |
| | CGTTAGCTTT AATTCAAACA TTAATTGGTA | |
| | ATCGTAAAAA A | |
| 252 | TTATTTT ATTCTATCTA TTTGGAAGTT | S. pneumoniae sero 31 |
| | TTCAAGGATA TGATAGTGGT GGTGCATTTA | PCR target |
| | TTATATTTTT ACTAATTTAT TTAAAATATA | |
| | TTTTATTCAA ATTTATAATT ATAAATATAC | |
| | CGCAATCCAA TTTAATAAGA GCATTTACAA | |
| | ACATAGGTAT TTTAAATACG GTCATTTTAT | |
| | TAGCAGAAGT TTTAAGTCAC GGACATATTA | |
| | ATTTATTTGT GAATCATTAT ACTTTGGCGC | |
| | AGAAAATAGA AACCTTAAAT AAGGTTGGGA | |
| | CAAACCTTGC GGTTTTACGT GGGGGGTTTG | |
| | AAAATCCCTT AGTGACATCT GTAATGCTAT | |
| | CTTCTACATT ATTATTTTTT ATGACTATTG | |
| | AGAAGGCTCT CTTACGTAAT ATACTAATTA | |
| | TGTCTAACTT ATTTTTGATT ATGGCAACAG | |
| | AAAAAAGAAC AGGAATTCTA ATTAGTATCG | |
| | CGTTGTTGTT CTGTTACTAT TTCAGAAAAA | |
| | ATTTAAAAAC AAAGAGTGTT AGTAAGTTTA | |
| | TAATAAAATT CTTGGGAGGA TTGTTTTTCT | |
| | TGGGATTTGC TTTATTAGCC ATAAATATGA | |
| | TAACAATTTC TGGTCGTAGC ATTTCTCAGA | |
| | TGATAATAGA GAGGTTTTCA TCATTAAGTA | |
| | GTGGTTCAGA CTTTTCTGCT ATACATAGGT | |
| | CAATGGCTTT TAAAATAGGC ATAGAAATAA | |
| | TTTGGAGTAG GAATATATTG AATATATTAT | |
| | TCGGTAATGG TTTCTACTTT TTGC | |
| 152 | ATGTATT CTATATCAAT AATTTGGAAT | S. pneumoniae sero 15c |
| | TTTTTAATTA GTGGCTTACC TATTCAAGTG | PCR target |
| | TTGTTTTCAG ATTTGAGTAA GGCATTCAAT | |
| | TGGATATTAG CAGTATTTTT TTATAATTAT | |
| | TATTTGAAAA ATCCCATTAA CGTTGACAAG | |
| | ATAAAGAAAT ATATGTTTTA TAATTTCGCT | |
| | ATATTAGTTA TTATTGTTGC TTTATTCTAT | |
| | GTTCAAAGAG GCGCTAATGT AGTATTGTTT | |
| | GGAAGAAGCT TATTAGGTTG GGACGGATTC | |
| | GTATCAGCTA CCAGTTACGG AGTAAGATAT | |
| | GCAGGATTTT TAGAATATTC AACATTAAAT | |
| | GGGCAGTTGA TTCTTTTTTT GTTACCGTTA | |
| | ATTAGGTTAT TTAAACTTAG TTTTTTTACA | |
| | CAAGTAACTA TTCTTGCTTT TTTGCTACAG | |
| | GTTTTAGTAT TGAGTAAATC TAGAATAGCT | |
| | ATTATTGCTC TGATTATATA CATAGTATTT | |
| | GTAGTAATGG TTCAGATTAC TTCAATTAAT | |
| | AAGCGGATGA TTGTAGCGTT TTATCCAAC | |
| 153 | AAGGAGC CTATGATTTA ATAGATGCCA | S. pneumoniae sero 8 PCR |
| | TGAATCAAGC | target |
| | AGTGGCTATA AATCCTAATT TGCATTTGAC | |
| | AATGGCTGGA GATGGAGAAC TTGAAGACAT | |
| | ACGTCAAAAG ATATCAAATC TGAATTTGAC | |
| | TGATCATATT ACAATATATG ATTGGGTTAA | |
| | TCAGAGAGAT AAAAAAATAC TATTTCAAGC | |
| | TAATCAGACT TTGATATTGG CTTCTTATAA | |
| | TGAGGGGCTC CCGATGGCAA TATTGGAAGC | |
| | TATGGCTTCT GGATTAGCAA TCATATCAAC | |
| | GCCTGTAGGT GGCATACCTG AAATTATACA | |
| | CGAGGATAAT GGCTGGTTAA TTCAACC | |
| 154 | GTATGTTC CATTTGTTAT CAGGTGTAGA | S. pneumoniae sero 10a |
| | TTTACCATTA GTGTCGGCAG ACAAATTATT | PCR target |
| | TAAATTCTTT GATCAGAACA | |
| | AATCAAATAA TTTTTTGAGC ATGGTCTCTG | |
| | ATGAGATTTT GAAATCAAAT AAAGTGTATG | |
| | AGCGTGTAAA ATTTAGATAC TTATTTCCTA | |
| | GATTCTTAGC TAGAAATATT CAGAACAAAT | |
| | ATGTTCGTAA ATTTGTAGCA TATTACCGTA | |
| | AGCTAGAGAT TAAAATTCAA CGTTTAATGA | |
| | AAATAGATTG TTTTAAAAAA TATAACATGA | |
| | GATTGGGTTA TGCATCTAAC TGGGTATCTA | |
| | TTAATCAGGA TTTAGTTAGA ATAATACTAG | |
| | AAGAAGAGAA AAATATTGAA AAAATATTTA | |
| | AATATTCGAT AGTAAATGAT GAACTGTTCA | |
| | TTCCTACGAT AATGTATAAA TACAATTTGA | |
| | TGGAATCGTT ATATTCATCT TCACCGATAA | |
| | CAGATGCCCC AAATGATTTT CAAGGAAATC | |
| | TAAGATATAT CAATTGGTGG GATGGAGATC | |
| | CTCATACCTG GACTGACTCT GAGCATGATA | |
| | TAGAACAATT GAAGCGTGGT AAAGCTTTAG | |
| | GTCATAAGTT TTCTAGAAAA TTTGATTTAG | |
| | AGAAATATCC GAATCTTAAA GAAGAAATTC | |
| | TAATTATCAT AAATAGGACA | |
| 155 | TTCCCAT CTATCTTGAT GATGAACATA | S. pneumoniae sero 35f |
| | GTCGCTATTG TATTTTATTT AAAGCAAAAT | PCR target |
| | AAAATACAAT ATTGGCAATG GTTATTGTTA | |
| | TCTTTATCTG CTTATTGGCT GTATGATGAA | |
| | ACGGATTCTC GATTAACATT TTATAGTTCC | |
| | TGTATATTGT TGATATGCAG TTTATTAATA | |
| | AGATGGATCC CAGAACTATT CTCTAAGTTC | |
| | GGATATATAT TTAAAGCTTT TAAACTCACC | |
| | TTTATTATAA ATGCAGTTGT TAGTTTTTGG | |
| | GTCTCCTTTA CCTATCTCAA TTTGAGTCAT | |
| | TCTTATATCA ATAATCTTTT TTTAAAATTA | |
| | GACTATATAT TAGGTGGTCG TATATACTTG | |
| | ATGAATAAAT CGTTGAATTT GTATGGTTTT | |
| | GGATTATTCG GACGACCAGT TGAATGGAAT | |
| | GGAAATGGTC TGACCATTCA AGGAGTTAGA | |
| | AACTATCAGA CCTATCTGTA TGTTGATAAT | |
| | TTGTATGTTC AAATTTTACA AAAATTTGGT | |
| | TTACTCGCTC TAGGAATAAT GCTCCTAGTC | |
| | CTAACTTTAA CCTTGTTTAA | |
| 156 | T ATTTTTTATT ATAAATTATG CTTTTGTAAG | S. pneumoniae sero 34 |
| | AGGAGATTAT TTTCACCCAA CAATTATCTT | PCR target |
| | TAACTTTACT TTTTTGATAG CAACACTGTT | |
| | TTGCATTTTA AATGCTCAAG AGTATGCTAT | |
| | TGAGTTCAAT GGAGGTACAG TTTTTATTGT | |
| | GACGATTTCA ATGCTCGTTT TTACAATAAT | |
| | AACAGTATTA TCAAAGAAAA TATTTAAGAC | |
| | TAATACTTTC ACCTTTAAGA ATAAATTAAA | |
| | ATATCTATAT GTCAGTAAAA GTCTAATTTT | |
| | TACAATAATT ATTATTCAGA TTTTAAATCT | |
| | TATTTTCTTT TATCGATACG AACAAGCTTT | |
| | ATTCAGCGCT TATGTTGGCG GTCGTGGAAG | |
| | TTTCTCGCAA ATAATTAATA ACTATGACCA | |
| | ATTAGTAAAG TTTTTTACTG AAGACTTAGT | |
| | CGGATTGGGG GTCAGGTCTC CGTTTTT | |
| 157 | ATTCTATTTCTTACCCGCTACTCTATAGAATGGAG | S. pneumoniae sero 1 PCR |
| | TATATAAACTATGGTTATACAAAAGATTTGATTTT | target |
| | CATGCAATGGCTGGCTTTGGCAACTTTGTTGTTAA | |
| | CTCTACCTTCTAAAGCCATTGTGTTAAGCGAAAGG | |
| | AAAATAAACCTAAATAACAATAACTATTTATTATT | |
| | TCTCAATAATATGAATACGTTTATCACAACAGTAG | |
| | CAATTATATATCTATTAGGAAGTGGTTTTAGAAAT | |
| | AAAGGGGAAATATATTCAGGTGCCAATATTGTGAT | |
| | AATGTTAGTATTTTCTTTGGTTTATTTTATGATTC | |
| | TCATT | |
| 158 | CTCAACT TGGGAACTAC TGATTCGTGA | S. pneumoniae sero 17f |
| | TGATAATTCCAATGATCAAA CAAGAGCAAT | PCR target |
| | TATTAAAGAA TACGAAGAAA AAGATAGACG | |
| | GATTA~ATTG ATTGAAAACA TATCGGAATA | |
| | TCATGGAGCC TATTATAATT TTTGGGGGTT | |
| | ATTAAACGATGTTAGACAAA ACGAAAATCC | |
| | ATTTGATTTC TATATGTTTG CTGATCAGGA | |
| | TGATATCTGG GATGTGGACA AATTAGAACG | |
| | TTTAATTAGT TATTACTATA GAAAAGTTAA | |
| | AACAGAAGAACCGGTATTGA TTTACGCTGA | |
| | TATGCGGATT ATAGATGCTA ACGGTAAAGT | |
| | GATAGCTAAT AGTATGCATC AATTGATGGG | |
| | AATTCGGTAT ACCAATCCTA TCTCAACTTT | |
| | TATGGCTCATAAGGTTTATG GATGTAATAC | |
| | GTTATTTAAT CATGAATTAT TTGAAATCTT | |
| | ACCCCTCCTT CCATGTTATG CTCCAGAATT | |
| | AGCCTTCCTA TCACATGATA ACTTTACAAC | |
| | AAAAATTGCTGCATTGAAAG GGCATGTGTA | |
| | TTTTTATGAT GAGCCTACTA TGAGTTATAG | |
| | ACGATATGGG CATAATCTTA CAAGTAAACA | |
| | TGAGTATAAC TTTACATTCA AGCGAATCTT | |
| | AAAACGTATCTCGAAAATTG ATCAATTAGC | |
| | TAAAGATCAT GCCTTGACTT ACAAGCAGAC | |
| | CTTAGTCGCT ACAAATTTGT TACATCAACA | |
| | AACAAGTATT GACACT | |
| 159 | GGAT GAACTTATTT CTAAAGGAGC | S. pneumoniae sero 20 |
| | AACAGTTTTT CACCTGACAG CGACAAGTAA | PCR target |
| | AAATCCCATA CGTTATTATT GGGAAATGTA | |
| | TAGATTCTTC AAAGAATATG CATCTGATTA | |
| | TCAGGCAATT TGGGTTAATA TTAATAGCTT | |
| | AGCAAATATT GACTATTTAA AATTTGCAAG | |
| | ATATTTTGGT ATTCCGGTTC GAATTGTACA | |
| | CAGCCATAAT AGTCAAAATA TGGATACCAA | |
| | ACTTCGTGAA AAATTACATA ATCATAATAG | |
| | AGACAAAATT GAAAAGTGGG CAACCGATTT | |
| | CTGGGCTTGT TCCCACGAAG CTGCAAAATG | |
| | GTTTTATAAC GAAGAAACTA TTGCAAAAGT | |
| | TAAGATTATA CCAAATGCTA TTAATATGAA | |
| | TGCTTCTATT TTTTCTTCTG AGGCACGAGA | |
| | TAAAATTCGA ATTGATTATA CATTAGACAA | |
| | TAAATTTGTG CTAGGTCATG TAGGACGTTT | |
| | GCATTTTCAG AAGAATCAAG AATTTATGAT | |
| | AAGAGTTTTA GCTAAATTAC AGGAATTTAG | |
| | AGACGATGTC TGCTTAGTTT | |
| 160 | CACTAAA AAAAATACGA ATAATTAGTA | S. pneumoniae sero 15a |
| | CAGCTGCTGG AATATCTCTT CTTTTTTTAA | PCR target |
| | TTGCCTATAA ACTCATTGAGATAGTTTTAC | |
| | CTGATAATTT GTATACTTTT TTTAATGTTA | |
| | CAAAGGCATT ATCATATGAG AATAGGACAG | |
| | AGTTTGCGGG ACGAACAAAT ACTATTTCCT | |
| | TCCTATGGGA CAACTTATTTTATCATGATT | |
| | ATATTAGTGC TATATTTGGG AAAGGACTAG | |
| | GTTCATACTC TGTTAATTAT ATTTATGAAC | |
| | TTGGTATAAT GCTTGCAGAT GGTGGTTTTA | |
| | TTTCAGTGAT TTTGCTTTATTCATTTTTGT | |
| | TGTCATTATT CATACGGGGA ACTATTACTA | |
| | GAGGAAAAAA TAAGCAAAGT GAAAGACTAA | |
| | TTGTATCCAT CATAGCTTTT GTGGTGATGA | |
| | TTAGCATTAT TGTTTGGAATAGTACGTTCA | |
| | CTAGATCAAC TTATCTTGTA TTTTTCT | |
| 161 | TGGATTGGTGTAA AAAAGGACTA TCTCAGTCAT | S. pneumoniae sero 7c |
| | CTATTGTTAA AGTTTACGAC GGGATCAAGT | PCR target |
| | TACCTCAAGT ATTTCGTGAT AAAAAATGGT | |
| | TTAGAAATAA AAAAATAAAT ATTGTATTTG | |
| | TTGGTGGTTA CGATATTAAA AAAGCTCAGG | |
| | AGTTGTTTTT AAGCTATTTT TTAAAATTAC | |
| | CAAAAGAAAT ACAAATGCAA TATACTTTGA | |
| | CTTTCTATGG AACTCGAAAA TCTAAGTATA | |
| | TAAAGAAATT ACAAAACATG TCTCAACATC | |
| | TATGTTCTGA TCAAGTTAAA TTTCATA | |
| 162 | TTCGTTTT GATAGAGGAA AAGCATCAGT | S. pneumoniae sero 18f |
| | ACAGTGTGCT AATTGGATTG AAGAAAAAAT | PCR target |
| | AAAAAACAAT AAAGTTATTG ATTGAAATAA | |
| | TTATATTTAT TGAATGATAA ATAAGGAGGC | |
| | CTTTAGATGA AAAGAGTTAT AACATATGGT | |
| | ACATTTGATT TATTGCATTA TGGTCATATC | |
| | AATCTTTTGA AACGTGCTAA ACAGCTAGGT | |
| | GATTATTTGA TTGTAGTTGT TTCAAGTGAT | |
| | GAGTTTAATT TAAAAGAAAA GAATAAAGTA | |
| | TGTTACTTTA ACTACGAACA CAGAAAAAAT | |
| | TTAGTAGAAG CTATTCGATA TGTCGATTTA | |
| | GTAATCCCTG AAACTAGTTG GGAACAGAAA | |
| | AAGTCAGATG TTAAAGAGTA CCGTATTGAC | |
| | ACTT TT | |
| 163 | ATAT CTGGAAGTTT AGTGGGATAC | S. pneumoniae sero 5 PCR |
| | CTACACAACT TCTGATTATG CCTTTGTGGA | target |
| | TCCTGTTGGG AGTAGTGTCT ATTTTTTCTA | |
| | GAATTGACAT GGAACGATCA TTCCTATTTT | |
| | TTTTATTAAC AATAGGTTGT TTAATTAGCA | |
| | CTATTGCTTT GTTAGATATA GTTACGGGAG | |
| | TATCTTATGT CTTTAATGGT TTGTCTCAGC | |
| | AACTCTATTT GGCTGTGGGA ATTCTAGTTT | |
| | TACGCTACTG GAATGCTGAT GTGATTGTTC | |
| | ATTATTGGAA AATCATCACC ATGACTTTTT | |
| | TGGGAGCATG TTTGCTGATT TCAGTGGATA | |
| | TTTATTTTCA CTACTTTCAA GGACATACTT | |
| | TTTCAAATAT TCATTATGTT TATCGAGCTA | |
| | AGAATTCAGC GGCATCTA | |
| 164 | TATTAAAAAA GAGGATAGAC TGGTAGTGAC | S. pneumoniae sero 23F |
| | AGCAACGACA ATAGTCATCT CTTTACTGGC | PCR target |
| | GTTAACATTT TTTTTCAAAC ATATAATTAA | |
| | TAATAGTGAA TCATATAGCC ATCGAGTGTT | |
| | AGGTGTTGTG AATTTTTTTA AATATTATGA | |
| | ATCAGATACG TTTCATTTGT TTTTTGGGGA | |
| | TGCTGAATTA GCCTTTGGAA ATACGACGAA | |
| | GGGT TAT | |
| 165 | TTGA ATTACCGAAC ATAAATAATT | S. pneumoniae sero 6A |
| | TGTATTTTAT TCATCCCTATATCTGGGGGT | PCR target |
| | GTACGGCAGG CTTTAATCAT GCATTGTTAG | |
| | AGATGGTTCC TTCAGTTGAT ATTGATAAAG | |
| | ATTATTTATA TATAGAAAAA CTGGCTCATG | |
| | ATAGTTATTT TGCAAAGTTTGCACTAGAGT | |
| | ATGGGAAGGT GTTGTTCTGC CCTGAGCAAC | |
| | TGGTCTTGTA TCGAAGACAC GGACATAATG | |
| | TAACAACTAG TCATCATTTT AAATTATCTC | |
| | CGCTAAATAT TCTCAGAAAGGCTATT | |
| 166 | TTGA ATTACCGAAC ATAAATAATT | S. pneumoniae sero 6B |
| | TGTATTTTAT TCATGCCTATATCTGGGGGT | PCR target |
| | GTACGGCAGG CTTTAATCAT GCATTGTTAG | |
| | AGATGGTTCC TTCACTTGAT ATTGATAAAG | |
| | ATTATTTATA TATAGAAAAA CTGTCTCATG | |
| | ATAATTATTT TGCAAAGTTTGCACTAGAGT | |
| | ATGGGAAGGT GTTGTTCTCC CCTGAGCAAC | |
| | TGGTCTTGTA TCGAAGACAC GGACATAATG | |
| | TAACAACTAG TCATCATTTT AAATTATCTC | |
| | CGCTAAATAT TCTCAGAAAGGCTATT | |
| 167 | GCGCAGAACTTTGAAGCGGTGGCGCAGTATCAGTT | phoE, Kiebsiella P RT-PCR |
| | CGACTTCGGTCTGCGTCCGTCCCTCGGCTATGTGC | target |
| | TGTCGAAAGGGAAGGATATCGAAGGGGTGGGGAGT | |
| | GAAGATCTGG | |
| 168 | AAAGCAACTATCGCGGCTACAGCTGGGATTGCGGT | p60, Lysteria RT-PCR target |
| | ACAGCATTTGCTGCTCCAACAATCGCATCCGCAA | |
| | GCACTGTAGTAGTCGAAGCTGGTGATACTCTTTGG | |
| | GGTATCGCACAAAGTAAAG | |
| 169 | GGAATATTTCGCGCCACTGCCGGAAGCAACGCGTA | uidA, E. coli RT-PCR target |
| | AACTCGACCCGACGCGTCCGATCACCTGCGTCAAT | |
| | GTAATGTTCTGCGACGCTCACACCGATACCATCAG | |
| | CG | |
| 170 | TGCAGATATCAATCTTATTTATCCTGAGACAACAC | sip, S. agalactiae RT-PCR |
| | TGACAGTAACTTACGATCACAAGAGTCATACTGCC | target |
| | ACTTCAATGAAAATAGAAGCACCAGCAACAAATGC | |
| | TGCTGGTCAAACA | |

1. Method for detecting the presence and/or the serogroup of a pathogen selected from the group consisting of N. meningitidis, H. influenzae, S. pneumoniae or Adenovirus in a biological sample, comprising the following steps:
a) incubating a first aliquot of the sample under conditions such as to enable the amplification and the revelation of specific target regions of the genoma of said pathogens, if present in the sample, wherein the target regions are comprised in:
SEQ ID 119 of the ctrA gene of N. meningitidis, and
SEQ ID 120 of the P2 gene or SEQ ID 121 of the bex gene of H. influenzae and
SEQ ID 122 of the lyt gene or SEQ ID 123 of the ply gene of S. pneumoniae and
SEQ ID 124 of Adenovirus;
and/or,
b) if the sample is postive for N. meningitidis, incubating a second aliquot of the sample under conditions such as to enable the aplification of specific serotyping target regions of the genoma of N. meningitidis, wherein the target regions are comprised in:
SEQ ID 125 for serotype B of N. meningitidis,
SEQ ID 126 for serotype C of N. meningitidis,
SEQ ID 127 for serotype W135 of N. meningitidis,
SEQ ID 128 for serotype W of N. meningitidis,
SEQ ID 129 for serotype Y of N. meningitidis,
SEQ ID 130 for serotype A of N. meningitidis,
c) revealing the amplification;
b′) if the sample is positive for H. influenzae, incubating a second aliquot of the sample under conditions such as to enable the amplification of region SEQ ID 131 of the genoma of H. influenzae for revealing capsulated H. influenzae, and
c′) revealing the amplification;
d′) if the sample is positive for the revelation of capsulated H. influenzae, incubating a third aliquot of the sample under conditions such as to enable the amplification of specific serotyping target regions of the genoma of H. influenzae wherein the target regions are comprised in:
SEQ ID 132 for the revelation of H. influenzae that are productors of beta-lactamase
SEQ ID 133 for the revelation of H. influenzae serotypes a, b, c, d, e, f;
SEQ ID 134 for the revelation of H. influenzae B type capsulated serotype;
b″) if the sample is positive for S. pneumonite, incubating a second aliquot of the sample under conditions such as to enable the amplification of specific serotyping target regions of the genoma of S. pneumoniae, wherein the target regions are comprised in:
SEQ ID 135 for serotype 19F of S. pneumoniae
SEQ ID 136 for serotype 22F of S. pneumoniae
SEQ ID 137 for serotype 3 of S. pneumoniae
SEQ ID 138 for serotype 6 of S. pneumoniae
SEQ ID 139 for serotype 19A of S. pneumoniae
SEQ ID 140 for serotype 9v of S. pneumoniae
SEQ ID 141 for serotype 4 of S. pneumoniae
SEQ ID 142 for serotype 14 of S. pneumoniae
SEQ ID 143 for serotype 12f of S. pneumoniae
SEQ ID 144 for serotype 7f of S. pneumoniae
SEQ ID 145 for serotype 11a of S. pneumoniae
SEQ ID 146 for serotype 33f of S. pneumoniae
SEQ ID 147 for serotype 16f of S. pneumoniae
SEQ ID 148 for serotype 35b of S. pneumoniae
SEQ ID 149 for serotype 18f of S. pneumoniae
SEQ ID 150 for serotype 38 of S. pneumoniae
SEQ ID 151 for serotype 31 of S. pneumoniae
SEQ ID 152 for serotype 15c of S. pneumoniae
SEQ ID 153 for serotype 8 of S. pneumoniae
SEQ ID 154 for serotype 10A of S. pneumoniae
SEQ ID 155 for serotype 35f of S. pneumoniae
SEQ ID 156 for serotype 34 of S. pneumonite
SEQ ID 157 for serotype 1 of S. pneumoniae
SEQ ID 158 for serotype 17f of S. pneumoniae
SEQ ID 159 for serotype 20 of S. pneumoniae
SEQ ID 160 for serotype 15a of S. pneumoniae
SEQ ID 161 for serotype 7c of S. pneumoniae
SEQ ID 162 for serotype 18f of S. pneumoniae
SEQ ID 163 for serotype 5 of S. pneumoniae
SEQ ID 164 for serotype 23F of S. pneumoniae
c″) highlighting the amplification;
d″) if the sample is positive for serotype 6 of S. pneumoniae, incubating a third aliquot of the sample under conditions such as to enable the amplification of the regions SEQ ID 165 for serotype 6a or SEQ ID 166 for serotype 6b of S. pneumoniae
e″) highlighting the amplification.

2. Method for detecting the presence and/or the serogroup of a pathogen selected from the group consisting of N. meningitidis, H. influenzae, S. pneumoniae or Adenovirus in a biological sample according to claim 1 wherein the target regions are comprised in:
from nt. 21 to nt 131 of SEQ ID 119 of the ctrA gene of N. meningitidis, and
from nt 21 to nt 171 of SEQ ID 120 of the P2 gene or from nt 21 to nt 120 of SEQ ID 121 of the bex gene of H. influenzae and
from nt 21 to nt 121 of SEQ ID 122 of the lyt gene or from nt 21 to nt 101 of SEQ ID 123 of the ply gene of S. pneumoniae and
from nt 21 to nt 116 of SEQ ID 124 of Adenovirus.

3. Method for detecting the presence and/or the serogroup of a pathogen selected from the group consisting of N. meningitidis, H. influenzae, S. pneumoniae or Adenovirus in a biological sample according to claim 1 wherein,
if the sample is positive for N. meningitidis, the specific serotyping target regions of the genoma of N. meningitidis are comprised in:
from nt 21 to nt 477 of SEQ ID 125 for serotype B of N. meningitidis,
from nt 21 to nt 462 of SEQ ID 126 for serotype C of N. meningitidis,
from nt 21 to nt 718 of SEQ ID 127 for serotype W135 of N. meningitidis,
from nt 21 to nt 140 of SEQ ID 128 for serotype W of N. meningitidis,
from nt 21 to nt 140 of SEQ ID 129 for serotype Y of N. meningitidis,
from nt 21 to nt 415 of SEQ ID 130 for serotype A of N. meningitidis,
if the sample is positive for H. influenzae, the target region for revealing capsulated H. influenzae is comprised within the region from nt 21 to nt 121 of SEQ ID 131;
if the sample is positive for the revelation of capsulated H. influenzae, the specific serotyping target regions are comprised in:
from nt 21 to nt 477 of SEQ ID 132 for the revelation of H. influenzae that are productors of beta-lactamase
from nt 21 to nt 357 of SEQ ID 133 for the revelation of H. influenzae serotypes a, b, c, d, e, f;
from nt 21 to nt 263 of SEQ ID 134 for the revelation of H. influenzae B type capsulated serotype;
if the sample is positive for S. pneumonite, the specific serotyping target regions of the genoma of S. pneumoniae are comprised in:
from nt 21 to nt 149 of SEQ ID 135 for serotype 19F of S. pneumoniae
from nt 21 to nt 663 of SEQ ID 136 for serotype 22F of S. pneumoniae
from nt 21 to nt 391 of SEQ ID 137 for serotype 3 of S. pneumoniae
from nt 21 to nt 240 of SEQ ID 138 for serotype 6 of S. pneumoniae
from nt 21 to nt 498 of SEQ ID 139 for serotype 19A of S. pneumoniae
from nt 21 to nt 527 of SEQ ID 140 for serotype 9v of S. pneumoniae
from nt 21 to nt 350 of SEQ ID 141 for serotype 4 of S. pneumoniae
from nt 21 to nt 284 of SEQ ID 142 for serotype 14 of S. pneumoniae
from nt 21 to nt 396 of SEQ ID 143 for serotype 12f of S. pneumoniae
from nt 21 to nt 846 of SEQ ID 144 for serotype 7f of S. pneumoniae
from nt 21 to nt 483 of SEQ ID 145 for serotype 11 a of S. pneumoniae
from nt 21 to nt 358 of SEQ ID 146 for serotype 33f of S. pneumoniae
from nt 21 to nt 1008 of SEQ ID 147 for serotype 16f of S. pneumoniae
from nt 21 to nt 697 of SEQ ID 148 for serotype 35b of S. pneumoniae
from nt 21 to nt 593 of SEQ ID 149 for serotype 18f of S. pneumoniae
from nt 21 to nt 594 of SEQ ID 150 for serotype 38 of S. pneumoniae
from nt 21 to nt 721 of SEQ ID 151 for serotype 31 of S. pneumoniae
from nt 21 to nt 516 of SEQ ID 152 for serotype 15c of S. pneumoniae
from nt 21 to nt 314 of SEQ ID 153 for serotype 8 of S. pneumoniae
from nt 21 to nt 648 of SEQ ID 154 for serotype 10A of S. pneumoniae
from nt 21 to nt 537 of SEQ ID 155 for serotype 35f of S. pneumoniae
from nt 21 to nt 428 of SEQ ID 156 for serotype 34 of S. pneumonite
from nt 21 to nt 300 of SEQ ID 157 for serotype 1 of S. pneumoniae
from nt 21 to nt 713 of SEQ ID 158 for serotype 17f of S. pneumoniae
from nt 21 to nt 534 of SEQ ID 159 for serotype 20 of S. pneumoniae
from nt 21 to nt 454 of SEQ ID 160 for serotype 15a of S. pneumoniae
from nt 21 to nt 280 of SEQ ID 161 for serotype 7c of S. pneumoniae
from nt 21 to nt 374 of SEQ ID 162 for serotype 18f of S. pneumoniae
from nt 21 to nt 382 of SEQ ID 163 for serotype 5 of S. pneumoniae
from nt 21 to nt 197 of SEQ ID 164 for serotype 23F of S. Pneumonia;
if the sample is positive for serotype 6 of S. pneumoniae, the target regions enabling the discrimination between serotype 6a and 6b are, from nt 21 to nt 270 of SEQ ID 165 and from nt 21 to nt 270 of SEQ ID 166, respectively.

4. Method for detecting the presence and/or the serogroup of a pathogen selected from the group consisting of N. meningitidis, H. influenzae, S. pneumoniae or Adenovirus in a biological sample according to claim 1 wherein the amplification and revelation of the specific regions comprised in SEQ ID 119 of the ctrA gene of N. meningitidis, and in SEQ ID 122 of the lyt gene or in SEQ ID 123 of the ply gene of S. pneumoniae occurs in a single first reaction environment; and the amplification and the revelation of the specific regions comprised in SEQ ID 120 of the P2 gene or in SEQ ID 121 of the bex gene of H. influenzae, and in SEQ ID 124 of Adenovirus occurs in a single second reaction environment.

5. Method for detecting the presence and/or the serogroup of a pathogen selected from the group consisting of N. meningitidis, H. influenzae, S. pneumoniae or Adenovirus in a biological sample according to claim 1 wherein if the sample is positive for S. pneumoniae, the second aliquot is incubated under conditions such as to enable the amplification of specific serotyping target regions of the genoma of S. pneumoniae, in a single first reaction environment for the sequences comprised in:
SEQ ID 135 for serotype 19F, SEQ ID 138 for serotype 6, SEQ ID 140 for serotype 9v, SEQ ID 141 for serotype 4, SEQ ID 142 for serotype 14, SEQ ID 163 for serotype 5;
in a single second reaction environment for the sequences comprised in:
SEQ ID 136 for serotype 22F, SEQ ID 138 for serotype 6, SEQ ID 137 for serotype 3, SEQ ID 139 for serotype 19A;
in a single third reaction environment for the sequences comprised in:
SEQ ID 140 for serotype 9v, SEQ ID 141 for serotype 4, SEQ ID 142 for serotype 14, SEQ ID 143 for serotype 12f;
in a single fourth reaction environment for the sequences comprised in:
SEQ ID 144 for serotype 7f, SEQ ID 145 for serotype 11A, SEQ ID 146 for serotype 33f;
in a single fifth reaction environment for the sequences comprised in:
SEQ ID 147 for serotype 16f, SEQ ID 148 for serotype 35b, SEQ ID 149 for serotype 18f, SEQ ID 150 for serotype 38;
in a single sixth reaction environment for the sequences comprised in:
SEQ ID 151 for serotype 31, SEQ ID 152 for serotype 15c, SEQ ID 153 for serotype 8, SEQ ID 154 for serotype 10A;
in a single seventh reaction environment for the sequences comprised in:
SEQ ID 155 for serotype 35f, SEQ ID 156 for serotype 34, SEQ ID 157 for serotype 1, SEQ ID 158 for serotype 17f;
in a single eighth reaction environment for the sequences comprised in:
SEQ ID 159 for serotype 20, SEQ ID 160 for serotype 15°, SEQ ID 161 for serotype 7c, SEQ ID 162 for serotype 18f;
in a single ninth reaction environment for the sequences comprised in:
SEQ ID 163 for serotype 5, SEQ ID 164 for serotype 23F.

6. Method for detecting the presence and/or the serogroup of a pathogen selected from the group consisting of N. meningitidis, H. influenzae, S. pneumoniae or Adenovirus in a biological sample according to claim 1 wherein the reaction of amplification and revelation of step a) occurs by RT-PCR.

7. Method for detecting the presence and/or the serogroup of a pathogen selected from the group consisting of N. meningitidis, H. influenzae, S. pneumoniae or Adenovirus in a biological sample according to the foregoing claims claim 1 wherein the reactions of amplification and revelation of the steps from b) to e″) occur by PCR and revelation of the amplificate by chromatography.

8. Method for detecting the presence and/or the serogroup of a pathogen selected from the group consisting of N. meningitidis, H. influenzae, S. pneumoniae or Adenovirus in a biological sample according to the foregoing claims claim 1 wherein the sample is not pre-incubated to increase the pathogen load.

9. Kit for detecting the presence and/or the serogroup of a pathogen selected from the group consisting of N. meningitidis, H. influenzae, S. pneumoniae or Adenovirus in a biological sample comprising primer and probe oligonucleotides capable of amplifying the target reasons cited in claim 1 and control target regions.

10. Kit according to claim 9 wherein the primers and probes for N. meningitidis and S. pneumoniae are in a single first reaction environment and the primers and probes for H. influenzae and Adenovirus are in a single second reaction environment.

11. Kit according to claim 9 wherein:
the primers for SEQ ID 119 are SEQ ID 1 and SEQ ID 2 and the probe is SEQ ID 91;
the primers for SEQ ID 120 are SEQ ID 116 and SEQ ID 117 and the probe is SEQ ID 118; o the primers for SEQ ID 121 are SEQ ID 3 and SEQ ID 4 and the probe is SEQ ID 92;
the primers for SEQ ID 122 are SEQ ID 5 and SEQ ID 6 and the probe is SEQ ID 93; o the primers for SEQ ID 123 are SEQ ID 94 and SEQ ID 95 and the probe is SEQ ID 96;
the primers for SEQ ID 124 are SEQ ID 99 and SEQ ID 100 and the probe is SEQ ID 101.

12. Kit according to claim 9 for the amplification of specific serotyping target regions of the genoma of N. meningitidis, wherein:
the primers for SEQ ID 125 are SEQ ID 9 and SEQ ID 10;
the primers for SEQ ID 126 are SEQ ID 11 and SEQ ID 12;
the primers for SEQ ID 127 are SEQ ID 13 and SEQ ID 14;
the primers for SEQ ID 128 are SEQ ID 15 and SEQ ID 15;
the primers for SEQ ID 129 are SEQ ID 17 and SEQ ID 18;
the primers for SEQ ID 130 are SEQ ID 19 and SEQ ID 20,
the primers for the control region are SEQ ID 1 and SEQ ID 2, or SEQ ID 7 and SEQ ID 8.

13. Kit according to claim 9 for the amplification of specific serotyping target regions of the genoma of H. Influenzae, wherein:
the primers for SEQ ID 131 are SEQ ID 97 and SEQ ID 98;
the primers for SEQ ID 132 are SEQ ID 23 and SEQ ID 24;
the primers for SEQ ID 133 are SEQ ID 25 and SEQ ID 26;
the primers for SEQ ID 134 are SEQ ID 27 and SEQ ID 28;
the primers for the control region are SEQ ID 21 and SEQ ID 22.

14. Kit according to claim 9 for the amplification of specific serotyping target regions of the genoma of S. pneumoniae, wherein:
the primers for SEQ ID 135 are SEQ ID 31 and SEQ ID 31;
the primers for SEQ ID 136 are SEQ ID 33 and SEQ ID 34;
the primers for SEQ ID 137 are SEQ ID 35 and SEQ ID 36;
the primers for SEQ ID 138 are SEQ ID 37 and SEQ ID 38;
the primers for SEQ ID 139 are SEQ ID 39 and SEQ ID 40;
the primers for SEQ ID 140 are SEQ ID 41 and SEQ ID 42;
the primers for SEQ ID 141 are SEQ ID 43 and SEQ ID 44;
the primers for SEQ ID 142 are SEQ ID 45 and SEQ ID 46;
the primers for SEQ ID 143 are SEQ ID 47 and SEQ ID 48;
the primers for SEQ ID 144 are SEQ ID 49 and SEQ ID 50;
the primers for SEQ ID 145 are SEQ ID 51 and SEQ ID 52;
the primers for SEQ ID 146 are SEQ ID 53 and SEQ ID 54;
the primers for SEQ ID 147 are SEQ ID 55 and SEQ ID 56;
the primers for SEQ ID 148 are SEQ ID 57 and SEQ ID 58;
the primers for SEQ ID 149 are SEQ ID 59 and SEQ ID 60;
the primers for SEQ ID 150 are SEQ ID 61 and SEQ ID 62;
the primers for SEQ ID 151 are SEQ ID 63 and SEQ ID 64;
the primers for SEQ ID 152 are SEQ ID 65 and SEQ ID 66;
the primers for SEQ ID 153 are SEQ ID 67 and SEQ ID 68;
the primers for SEQ ID 154 are SEQ ID 69 and SEQ ID 70;
the primers for SEQ ID 155 are SEQ ID 71 and SEQ ID 72;
the primers for SEQ ID 156 are SEQ ID 73 and SEQ ID 74;
the primers for SEQ ID 157 are SEQ ID 75 and SEQ ID 76;
the primers for SEQ ID 158 are SEQ ID 77 and SEQ ID 78;
the primers for SEQ ID 159 are SEQ ID 79 and SEQ ID 80;
the primers for SEQ ID 160 are SEQ ID 81 and SEQ ID 82;
the primers for SEQ ID 161 are SEQ ID 83 and SEQ ID 84;
the primers for SEQ ID 162 are SEQ ID 85 and SEQ ID 86;
the primers for SEQ ID 163 are SEQ ID 87 and SEQ ID 88;
the primers for SEQ ID 164 are SEQ ID 89 and SEQ ID 90;
the primers for SEQ ID 165 are SEQ ID 114 and SEQ ID 115;
the primers for SEQ ID 166 are SEQ ID 114 and SEQ ID 115;
the primers for the control region are SEQ ID 29 and SEQ ID 30,
and wherein said primers are, optionally, partially grouped in a plurality of reaction environments.

15. Kit for detecting the presence and the serogroup of a pathogen selected from the group consisting of N. meningitidis, H. influenzae, S. pneumoniae or Adenovirus in a biological sample comprising the kit according to claims 9.

16. Method for detecting the presence and/or the serogroup of a pathogen selected from the group consisting of Klebsiella pneumoniae, Lysteria monocytogenes, E. coli, S. agalactiae in a biological sample comprising the following steps:
a) incubating an aliquot of the sample under conditions such as to enable the amplification and revelation of specific target regions of the genoma of said pathogens, if present in the sample, wherein the target regions are comprised in:
SEQ ID 167 of the phoE gene of Klebsiella pneumoniae,
SEQ ID 168 of the iap gene of Lysteria monocytogenes,
SEQ ID 169 of the uidA gene of E. coli,
SEQ ID 170 of the sip gene of S. agalactiae.

17. Method for detecting the presence and/or the serogroup of a pathogen selected from the group consisting of Klebsiella pneumoniae, Lysteria monocytogenes, E. coli, S. agalactiae in a biological sample according to claim 16 wherein the target regions are comprised in:
from nt 21 to nt 95 of SEQ ID 167 of the phoE gene of Klebsiella pneumoniae,
from nt 21 to nt 104 of SEQ ID 168 of the iap gene of Lysteria monocytogenes,
from nt 21 to nt 87 of SEQ ID 169 of the uidA gene of E. coli,
from nt 21 to nt 98 of SEQ ID 170 of the sip gene of S. agalactiae.

18. Method for detecting the presence and/or the serogroup of a pathogen selected from the group consisting of Klebsiella pneumoniae, Lysteria monocytogenes, E. coli, S. agalactiae in a biological sample according to claim 16 wherein the amplification and revelation of the specific regions comprised in SEQ ID 167 of the phoE gene of Klebsiella pneumoniae, and in SEQ ID 169 of the uidA gene of E. coli occurs in a single first reaction environment; and the amplification and revelation of the specific regions comprised in SEQ ID 168 of the iap gene of Lysteria monocytogenes, and in SEQ ID 170 of the sip gene of S. agalactiae occurs in a single second reaction environment.

19. Method for detecting the presence and/or the serogroup of a pathogen selected from the group consisting of Klebsiella pneumoniae, Lysteria monocytogenes, E. coli, S. agalactiae in a biological sample according to claim 16 wherein the sample is not pre-incubated to increase the pathogen load.

20. Kit for detecting the presence and/or the serogroup of a pathogen selected from the group consisting of Klebsiella pneumoniae, Lysteria monocytogenes, E. coli, S. agalactiae in a biological sample comprising primer and probe oligonucleotides capable of amplifying the target regions cited in claim 16.

21. Kit according to claim 20 wherein the primers and probes for Klebsiella pneumonia and E. coli are in a single first reaction environment and the primers and probes for Lysteria monocytogenes and S. agalactiae are in a single second reaction environment.

22. Kit according to claim 20 wherein:
the primers for SEQ ID 167 are SEQ ID 102 and SEQ ID 103 and the probe is SEQ ID 104;
the primers for SEQ ID 168 are SEQ ID 105 and SEQ ID 106 and the probe is SEQ ID 107;
the primers for SEQ ID 169 are SEQ ID 108 and SEQ ID 109 and the probe is SEQ ID 110;
the primers for SEQ ID 170 are SEQ ID 111 and SEQ ID 112 and the probe is SEQ ID 113.
